(19) United States
(12) Patent Application Publication
(10) Pub. No.: US 2024/0170093 A1
(43) Pub. Date: May 23, 2024

(54) DETECTION OF MICRO-ORGANISMS

(71) Applicant: UNIVERSITY OF LANCASTER, Lancashire (GB)

(72) Inventors: Carlos Alejandro Meza Ramirez, Lancashire (GB); Craig Williams, Lancashre (GB); Ihtesham ur Rehman, West Yorkshire (GB)

(21) Appl. No.: 18/283,996

(22) PCT Filed: Mar. 31, 2022
(86) PCT No.: PCT/GB2022/050822

§ 371 Date: Sep. 25, 2023

(30) Foreign Application Priority Data
Mar. 31, 2021 (GB) 2104613.1
May 14, 2021 (GB) 2106955.4

(51) Int. Cl. G16B 15/00; G01N 21/3577; G01N 21/65; G06N 20/00; G16B 40/20

(52) U.S. Cl. CPC G16B 15/00; G01N 21/3577; G01N 21/65; G06N 20/00; G16B 40/20

(57) ABSTRACT

Methods, apparatus, a system, a computer program and a computer-readable data carrier are disclosed. A method comprises providing spectroscopic data associated with at least one microorganism, and obtained via a Raman or Infrared spectroscopy technique, as an input into at least one machine learning module, and responsive to providing the spectroscopic data, providing at least one trained machine learning model, wherein the trained machine learning model is configured to identify at least one microorganism in a biological sample.

### FIELD OF THE INVENTION

Aspects of the present invention provide methods of identifying and characterizing one or more types of micro-organisms present in a biological sample and methods of training machine learning modules to provide trained machine learning models to identify micro-organisms in a biological sample. Certain embodiments of the present invention enable microorganisms to be detected and identified and resistance mechanisms identified by applying machine learning algorithms (programming and training of data) together with vibrational spectroscopy (Raman spectroscopy and Infrared spectroscopy). The spectra obtained can be manipulated as numerical data.

### BACKGROUND TO THE INVENTION

Extreme resistance to antimicrobials is a hallmark of chronic biofilm-based infections. Biofilms are a structured consortium of microbial cells encased in a matrix of self-produced extracellular polymeric substances (EPS) that are capable of co-ordinated behaviour. No current antimicrobial therapy can eradicate mature biofilms. Suppression of infection is possible with prolonged treatment and high doses of antimicrobials. In addition, detection of infections in hospitals is time consuming and costly. The overall burden of biofilm infections is significant with most of hospital-acquired infections related to biofilms contributing to a direct cost of ˜£1 billion per year in the UK alone.

In addition to conventional resistance mechanisms such as upregulation of multidrug efflux pumps and horizontal transfer of resistance genes, the resistance of mature biofilms to antimicrobials is attributed to the EPS matrix—the dark matter of the biofilms. The matrix is a highly hydrated mixture where chemical species including proteins, polysaccharides and DNA can diffuse and react. The composition of the matrix is dynamic and known to change when biofilms are subject to external stressors.

Several bacterial species have demonstrated their ability to form biofilms both in vitro and in vivo. In their biofilm state, bacteria present differential metabolic and physiological functions often rendering them more virulent and resistant to antibiotics. Understanding aspect of infections such as bacterial cell-cell communication, biofilm formation, mechanical stability, survival and chemical nature of EPS will potentially lead to the development of novel therapeutics.

Using current methods it takes on average 48 hours, from 12-72 hours to detect/identify a bacterium and determine its sensitivity to antimicrobials. There is thus a need to detect/identify microorganisms in biofilms or other biological systems more quickly than is possible using current methods. It is important both to identify specific micro-organisms present in an infection and identify their sensitivity to antimicrobials to be able to direct treatment. Thus, rapid detection and identification of bacteria and identifying resistance to antimicrobials is important for managing infections.

COVID-19 also requires robust, easy-to-use Point-of-Care (POC) and immediately deployable screening systems providing large-scale monitoring for occurrences of the virus, to prevent its spread or recurrence. Current methods for identifying the presence of SARS-COV-2 are too slow and logistically cumbersome. Current methods for identifying the presence of SARS-COV-2 and its variants also involve expensive and complicated supply chains, involving reagents, injection moulded cartridges, are limited to a specific virus and have low sensitivity. The requirement to stockpile the test cartridges is also a major disadvantage. Both these require environmentally unfriendly single-use plastics consumables. There is thus a desire to address the need for near-instantaneous testing for COVID-19 virus, that fits into the existing clinical workflow, using current nasopharyngeal sample swabs.

Certain embodiments of the present invention aim to at least partly overcome one or more of the problems associated with the prior art.

Certain embodiments of the present invention aim to provide a method of identifying a micro-organism or multiple microorganisms e.g. a micro-organism species.

Certain embodiments of the present invention aim to provide a trained machine learning model that can identify one or more microorganisms in a biological sample.

Certain embodiments of the present invention aim to train one or more machine learning modules to provide trained machine learning models to identify microorganisms in a biological sample.

Certain embodiments of the present invention aim to utilise spectrographic data, obtained by inspecting a biological sample with a Raman or Infrared spectroscopy technique, to identify micro-organisms present in the sample.

Certain embodiments of the present invention aim to utilise spectrographic data, obtained by inspecting a biological sample with a Raman or Infrared spectroscopy technique, to train machine learning modules and thereby provide trained machine learning models that can detect/identify micro-organisms present in a biological sample.

Certain embodiments of the present invention use Infrared and Raman spectroscopy combined with chemometric analysis to identify microorganisms, understand the dynamics of EPS accumulation that are known to increase the stability of biofilms.

Certain embodiments of the present invention aim to identify the presence of microorganisms in a biological sample in near real time by utilising machine learning models (e.g. stored in the cloud) that are able to receive data from a remote location, process the data and provide an identification result to the remote location.

### SUMMARY OF THE INVENTION

In a first aspect of the present invention there is provided a method of training at least one machine learning module to provide at least one trained machine learning model that is configured to identify at least one microorganism in a biological sample, the method comprising the steps of:
providing spectroscopic data associated with at least one microorganism, and obtained via a Raman or Infrared spectroscopy technique, as an input into at least one machine learning module; and
responsive to providing the spectroscopic data, providing at least one trained machine learning model,
wherein the trained machine learning model is configured to identify at least one microorganism in a biological sample.

In certain embodiments, each machine learning module is a multinomial classifier. In certain embodiments, each machine learning module is one of a linear discriminant analysis module, a support vector machine module, a logistic regression module, a K nearest neighbours module, a random forest module, an artificial neural network module or a convolutional neural network module.

In certain embodiments, the method further comprises:
providing spectroscopic data as an input into a plurality of machine learning modules; and
responsive to providing the spectroscopic data, providing a plurality of trained machine learning models,
wherein each trained machine learning model is configured to identify at least one microorganism in a biological sample.

In certain embodiments, the plurality of machine learning modules comprises a linear discriminant analysis module, a support vector machine module, a logistic regression module, a K nearest neighbours module, a random forest module, optionally an artificial neural network module, and optionally a convolutional neural network module; the method further comprising:
providing spectroscopic data as an input into each of the plurality of machine learning modules; and
responsive to providing the spectroscopic data, providing a trained linear discriminant analysis based model, a trained support vector machine based model, a trained logistic regression based model, a trained K nearest neighbours based model, a trained random forest based model, optionally a trained artificial neural network based model and optionally a trained convolutional neural network based model.

In certain embodiments, the method further comprises:
determining at least one score indicative of an identification accuracy of a selected microorganism for each of the plurality of trained machine learning models, wherein optionally the score is a specificity or sensitivity or F1 score or negative predictive value (NPV) or positive predictive value (PPV) or negative predictive agreement (NPA) or positive predictive agreement (PPA).

In certain embodiments, the method further comprises:
responsive to comparing a plurality of scores, each score being associated with a respective one of the plurality of trained machine learning models, selecting a trained machine learning model having a score that is a highest score for the plurality of trained machine learning models, for the selected microorganism.

In certain embodiments, the at least one trained machine learning model is configured to identify a plurality of microorganisms. In certain embodiments, the at least one trained machine learning model is configured to identify a plurality of microorganisms simultaneously.

In certain embodiments, the method further comprises:
responsive to providing the spectroscopic data, determining a mapping between spectroscopic data, associated with a predetermined microorganism, and the predetermined microorganism; and
providing at least one trained machine learning model based on said mapping.

In certain embodiments, the method further comprises:
responsive to providing the spectroscopic data, determining a plurality of mappings between spectroscopic data, associated with each of a plurality of predetermined microorganisms, and each respective predetermined microorganism; and
   providing at least one trained machine learning model based on said mappings.

In certain embodiments, the method further comprises:
determining at least one parameter and/or a spatial mapping for the machine learning model based on the provided spectroscopic data.

In certain embodiments, the spatial mapping comprises comparing a spatial position in a feature space of newly input spectroscopic data to a spatial position in a feature space of previously input spectroscopic data.

In certain embodiments, the method further comprises:
providing the spectroscopic data as a feature matrix comprising a plurality of instances, each instance comprising spectroscopic data.

In certain embodiments, the plurality of instances comprises intensity values, associated with spectroscopic measurements of a plurality of microorganisms, at each of a plurality of wavelengths or wavenumbers.

In certain embodiments, each instance comprises intensity values, associated with a spectroscopic measurement of a predetermined microorganism, at each of a plurality of wavelengths or wavenumbers.

In certain embodiments, each instance of the feature matrix further comprises an encoded class label that indicates a type of microorganism associated with the spectroscopic data in that instance. In certain embodiments, the plurality of wavelengths or wavenumbers are in a range between 4000 cm⁻¹ and 50 cm⁻¹.

In certain embodiments, the range of wavelengths or wavenumbers comprises a high wavenumber region between 4000 cm⁻¹ and 2000 cm⁻¹, a fingerprint region between 1800 cm⁻¹ and 100 cm⁻¹, and a low wavenumber region between 400 cm⁻¹ and 50 cm⁻¹.

In certain embodiments, the at least one instance comprises intensity values, associated with a spectroscopic measurement of a predetermined microorganism, in the high wavenumber region or the fingerprint region or the low wavenumber region.

In certain embodiments, the method further comprises:
providing a trained machine learning model for at least one of or each of the high wavenumber region, the fingerprint region and the low wavenumber region.

In certain embodiments, the method further comprises:
prior to providing the spectrographic data as an input into the machine learning module, randomly shuffling the plurality of instances.

In certain embodiments, the method further comprises:
after randomly shuffling the instances and prior to providing the spectroscopic data as an input into the machine learning module, splitting the plurality of instances into a training dataset, a validation dataset and a test dataset.

In certain embodiments, the method further comprises:
providing the training dataset as an input into the machine learning module to provide an initial trained machine learning model.

In certain embodiments, the method further comprises:
providing the validation dataset as an input into the initial trained machine learning model; and
responsive to providing the validation dataset, determining one or more hyperparameters of the initial machine learning model to provide the trained machine learning model.

In certain embodiments, the method further comprises:
providing the test dataset as an input into the trained machine learning model to determine an identification accuracy of the trained machine learning model for one or more selected microorganisms.

In certain embodiments, the method further comprises:
after splitting the plurality of instances and prior to providing the spectroscopic data as an input into the machine learning module, normalising the spectroscopic data in each instance.

In certain embodiments, the method further comprises:
normalising the spectroscopic data via scaling intensity values of the spectroscopic data in each instance into a predetermined range.

In certain embodiments, the method further comprises:
after normalising the spectroscopic data and prior to providing the spectroscopic data as an input into the machine learning module, applying a principal components analysis to the spectroscopic data in the plurality of instances.

In certain embodiments, the method further comprises:
responsive to providing the spectroscopic data, determining one or more bio-markers associated with at least one predetermined microorganism via the machine learning module, wherein the trained machine learning model is at least partly based on the determined bio-markers;
the bio-markers including at least one of a spectral peak position, a spectral shift, a spectral shape, a spectral intensity, and a spectral area associated with a predetermined microorganism.

In certain embodiments, the biological sample is at least one bodily fluid of a patient that includes the at least one microorganism. In certain embodiments, the bodily fluid is one or more of: urine, saliva, whole blood, serum, cerebro spinal fluid, peritoneal fluid, sputum and pus.

In certain embodiments, the biological sample is obtained from a nasal and/or oropharyngeal swab.

In certain embodiments, the biological sample is taken from an environmental source.

In certain embodiments, the biological sample is taken from a food source.

In certain embodiments, the method further comprises:
providing the biological sample into a Raman or Infrared spectrometer to obtain the spectroscopic data.

In certain embodiments, the method further comprises:
providing the biological sample into a Raman spectrometer to obtain Raman spectroscopic data; and
providing the biological sample into an Infrared spectrometer to obtain Infrared spectroscopic data.

In certain embodiments, the method further comprises:
responsive to providing the Raman spectroscopic data as an input into at least one machine learning module, providing at least one trained machine learning model associated with the Raman spectroscopic data; and
responsive to providing the Infrared spectroscopic data as an input into at least one machine learning module, providing at least one trained machine learning model associated with the Infrared spectroscopic data.

In certain embodiments, the method further comprises:
providing spectroscopic data within a first spectral region from 4000-400 cm⁻¹ as an input into at least one machine learning module, and responsive to providing the spectroscopic data within the first spectral region, providing at least one trained machine learning model associated with the first spectral region.

In certain embodiments, the method further comprises:
providing spectroscopic data within a second spectral region from 4000-2500 cm⁻¹ as an input into at least one machine learning module, and responsive to providing the spectroscopic data within the second spectral region, providing at least one trained machine learning model associated with the second spectral region.

In certain embodiments, the method further comprises:
providing spectroscopic data within a third spectral region from 3800-2500 cm⁻¹ as an input into at least one machine learning module, and responsive to providing the spectroscopic data within the third spectral region, providing at least one trained machine learning model associated with the third spectral region.

In certain embodiments, the method further comprises:
providing spectroscopic data within a fourth spectral region from 1800-400 cm⁻¹ as an input into at least one machine learning module, and responsive to providing the spectroscopic data within the fourth spectral region, providing at least one trained machine learning model associated with the fourth spectral region.

In certain embodiments, the method further comprises:
providing spectroscopic data within a fifth spectral region from 1810-1700 cm⁻¹ as an input into at least one machine learning module, and responsive to providing the spectroscopic data within the fifth spectral region, providing at least one trained machine learning model associated with the fifth spectral region.

In certain embodiments, the method further comprises:
providing spectroscopic data within a sixth spectral region from 1590-1290 cm⁻¹ as an input into at least one machine learning module, and responsive to providing the spectroscopic data within the sixth spectral region, providing at least one trained machine learning model associated with the sixth spectral region.

In certain embodiments, the method further comprises:
providing spectroscopic data within a seventh spectral region from 1700-1600 cm⁻¹ as an input into at least one machine learning module, and responsive to providing the spectroscopic data within the seventh spectral region, providing at least one trained machine learning model associated with the seventh spectral region.

In certain embodiments, the method further comprises:
providing spectroscopic data within an eighth spectral region from 1600-1500 cm⁻¹ as an input into at least one machine learning module, and responsive to providing the spectroscopic data within the eighth spectral region, providing at least one trained machine learning model associated with the eighth spectral region.

In certain embodiments, the method further comprises:
providing spectroscopic data within a ninth spectral region from 1200-1000 cm⁻¹ as an input into at least one machine learning module, and responsive to providing the spectroscopic data within the ninth spectral region, providing at least one trained machine learning model associated with the ninth spectral region.

In certain embodiments, the method further comprises:
providing spectroscopic data within a tenth spectral region from 1000-400 cm⁻¹ as an input into at least one machine learning module, and responsive to providing the spectroscopic data within the tenth spectral region, providing at least one trained machine learning model associated with the tenth spectral region.

In certain embodiments, the method further comprises:
providing spectroscopic data within an eleventh spectral region from 1500-1200 cm⁻¹ as an input into at least one machine learning module, and responsive to providing the spectroscopic data within the eleventh spectral region, providing at least one trained machine learning model associated with the eleventh spectral region.

In certain embodiments, the method further comprises:
providing at least one machine learning model for each of the first to eleventh spectral regions.

In certain embodiments, the method further comprises:
the at least one trained machine learning model associated with the second spectral region (4000-2500 cm⁻¹) is configured to identify at least one virus in the biological sample, and wherein the biological sample is at least one bodily fluid of a patient that includes the at least one virus, the bodily fluid being one or more of: urine, saliva, whole blood, serum, cerebro spinal fluid, peritoneal fluid, nasopharyngeal aspirate, sputum and pus.

In certain embodiments, the Raman or Infrared spectroscopy technique is a micro-spectroscopy technique and/or a fibre optic probe spectroscopy technique.

In certain embodiments, the Raman or Infrared spectroscopy technique is one or more of a transmission, reflectance or absorbance spectroscopy technique.

In certain embodiments, the Infrared spectroscopy technique is a Fourier Transform Infrared spectroscopy technique and/or an Attenuated Total Reflectance Infrared spectroscopy technique.

In certain embodiments, the microorganism is a bacterial pathogen contained in one of the following Phylum:
1. Actinobacteria
2. Bacteroidetes
3. Firmicutes
4. Fusobacteria
5. Proteobacteria

In certain embodiments, the microorganism is a viral pathogen contained in one of the following orders:
1. Herpesvirales
2. Mononegavirales
3. Nidovirales
4. Picornavirales

In certain embodiments, the microorganism is a viral pathogen contained in one of the following families:
1. Adenovirus
2. Astroviridiae
3. Caliciviridiae
4. Flaviviridiae
5. Hepadnaviridae
6. Hepeviridiae
7. Orthomyxoviridiae
8. Reoviridiae
9. Coronaviridae

In certain embodiments, the microorganism is a fungal pathogen contained in one of more of the divisions:
1. Ascomycota
2. Basidiomycota

In certain embodiments, the microorganism is selected from at least one of:
Staphylococcus spp to species level (e.g., S aureus) and/or associated serotypes and/or antibiotic resistant variants;
Klebsiella spp to species level (e.g., K pneumoniae) and/or associated serotypes and/or antibiotic resistant variants;
Streptococcus spp to lancefield group level (e.g., Group A streptococcus) and/or associated serotypes and/or antibiotic resistant variants;
Pseudomonas spp to species level (e.g., Ps aeruginosa) and/or associated serotypes and/or antibiotic resistant variants;
Candida spp to species level (e.g., C albicans) and/or associated serotypes and/or antifugla resistant variants;
Escherichia spp to species level (e.g., E coli) and/or associated serotypes and/or antibiotic resistant variants;
Saccharomyces spp and/or associated serotypes;
Salmonella spp and/or associated serotypes;
Vibrio spp and/or associated serotypes;
Enterococcus spp and/or associated serotypes; and
SARS-COV-2 spp.

In certain embodiments, the method is for the detection of SARS-COV-2 in a sample. In certain embodiments, the method is for determining a variant of SARS-COV-2 in a sample. For example, the method may determine the presence of a SARS-COV-2 variant selected from B.1.1.7 (“Kent variant”), B.1.351 (“South African variant”) and P1 variant (“Brazilian variant”) in a sample. Aptly, the sample is taken from a subject suspected of suffering from a SARS-COV-2 infection.

In a further aspect of the present invention, there is provided a method of identifying at least one microorganism in a biological sample, comprising the steps of:
providing spectroscopic data associated with a biological sample, and obtained via a Raman or Infrared spectroscopy technique, as an input into at least one trained machine learning model; and
responsive to providing the spectroscopic data, identifying at least one microorganism in the biological sample.

In certain embodiments, the method comprises providing the spectroscopic data associated with the biological sample as an input into the at least one trained machine learning model trained via the method of the first aspect of the invention.

In certain embodiments, the method further comprises providing the spectroscopic data as an input into each of a plurality of trained machine learning models; and
responsive to providing the spectroscopic data, identifying at least one microorganism via each of the plurality of trained machine learning models.

In certain embodiments, the method further comprises:
providing the spectroscopic data as an input into each of the plurality of trained machine learning models simultaneously.

In certain embodiments, the method further comprises;
responsive to comparing a plurality of scores, each score being associated with a respective one of the plurality of trained machine learning models and being indicative of an identification accuracy of an expected microorganism in the biological sample, selecting a trained machine learning model having a score that is a highest score for the plurality of trained machine learning models, for identifying the at least one microorganism.

In certain embodiments, the method further comprises:
providing the spectroscopic data into a trained machine learning model that is a model with a highest score indicative of an identification accuracy associated with at least one expected type of microorganism in the biological sample.

In certain embodiments, the method further comprises:
prior to providing the spectroscopic data as an input into the trained machine learning model, normalising the spectroscopic data.

In certain embodiments, the method further comprises:
normalising the spectroscopic data via scaling intensity values of the spectroscopic data into a predetermined range.

In certain embodiments, the method further comprises:
after normalising the spectroscopic data and prior to providing the spectroscopic data as an input into the trained machine learning model, applying a principal components analysis to the spectroscopic data.

In certain embodiments, each trained machine learning model is a multinomial classifier.

In certain embodiments, the method further comprises:
responsive to providing the spectroscopic data, identifying a plurality of microorganisms in the biological sample.

In certain embodiments, the biological sample is at least one bodily fluid of a patient that includes the at least one microorganism. The biological sample may comprise a mixture of at least two bacteria or a mixture of at least one bacterium and at least one fungus or a mixture of at least two fungi or a mixture of at least one bacterium and at least one virus or a mixture of at least one fungus and at least one virus or a bacterial and fungal species present as a colony. The biological sample may be a direct biofluid from a patient mixed with at least one bacterium and/or at least one virus and/or an antibiotic resistant microorganism.

In certain embodiments, the biological sample is at least one bacterial or fungal culture derived from a sample from a patient that includes the at least one microorganism. The biological sample may comprise a mixture of at least two bacteria or a mixture of at least one bacterium and at least one fungus or a mixture of at least two fungi or a mixture of at least one bacterium.

In certain embodiments, the method further comprises:
providing the biological sample into a Raman or Infrared spectrometer to obtain the spectroscopic data. In certain embodiments, the Raman or Infrared spectroscopy technique is a micro-spectroscopy technique and/or a fibre optic probe spectroscopy technique. The Raman spectroscopy technique may be one or more of a reflectance spectroscopy technique. The Infrared spectroscopy technique may be a reflectance, transmission or absorbance spectroscopy technique. The Infrared spectroscopy technique may be a Fourier Transform Infrared spectroscopy technique and/or a Fourier Transform Infrared spectroscopy technique coupled with an Attenuated Total Reflectance Infrared spectroscopy technique.

In certain embodiments, the method further comprises:
providing the biological sample into a Raman spectrometer to obtain Raman spectroscopic data; and
providing the biological sample into an Infrared spectrometer to obtain Infrared spectroscopic data.

In certain embodiments, the method further comprises:
responsive to providing the Raman spectroscopic data as an input into at least one trained machine learning model, identifying at least one microorganism in the biological sample; and/or
responsive to providing the Infrared spectroscopic data as an input into at least one trained machine learning model, identifying at least one microorganism in the biological sample.

In certain embodiments, the method further comprises:
providing spectroscopic data within a first spectral region from 4000-400 cm⁻¹ as an input into at least one trained machine learning model associated with the first spectral region, and responsive to providing the spectroscopic data within the first spectral region, identifying at least one microorganism in the biological sample.

In certain embodiments, the method further comprises:
providing spectroscopic data within a second spectral region from 4000-2500 cm⁻¹ as an input into at least one trained machine learning model associated with the second spectral region, and responsive to providing the spectroscopic data within the second spectral region, identifying at least one microorganism in the biological sample.

In certain embodiments, the method further comprises:
providing spectroscopic data within a third spectral region from 3800-2500 cm⁻¹ as an input into at least one trained machine learning model associated with the third spectral region, and responsive to providing the spectroscopic data within the third spectral region, identifying at least one microorganism in the biological sample.

In certain embodiments, the method further comprises:
providing spectroscopic data within a fourth spectral region from 1800-400 cm⁻¹ as an input into at least one trained machine learning model associated with the fourth spectral region, and responsive to providing the spectroscopic data within the fourth spectral region, identifying at least one microorganism in the biological sample.

In certain embodiments, the method further comprises:
providing spectroscopic data within a fifth spectral region from 1810-1700 cm⁻¹ as an input into at least one trained machine learning model associated with the fifth spectral region, and responsive to providing the spectroscopic data within the fifth spectral region, identifying at least one microorganism in the biological sample.

In certain embodiments, the method further comprises:
providing spectroscopic data within a sixth spectral region from 1590-1290 cm⁻¹ as an input into at least one trained machine learning model associated with the sixth spectral region, and responsive to providing the spectroscopic data within the sixth spectral region, identifying at least one microorganism in the biological sample.

In certain embodiments, the method further comprises:
providing spectroscopic data within a seventh spectral region from 1700-1600 cm⁻¹ as an input into at least one trained machine learning model associated with the seventh spectral region, and responsive to providing the spectroscopic data within the seventh spectral region, identifying at least one microorganism in the biological sample.

In certain embodiments, the method further comprises:
providing spectroscopic data within an eighth spectral region from 1600-1500 cm⁻¹ as an input into at least one trained machine learning model associated with the eighth spectral region, and responsive to providing the spectroscopic data within the eighth spectral region, identifying at least one microorganism in the biological sample.

In certain embodiments, the method further comprises:
providing spectroscopic data within a ninth spectral region from 1200-1000 cm⁻¹ as an input into at least one trained machine learning model associated with the ninth spectral region, and responsive to providing the spectroscopic data within the ninth spectral region, identifying at least one microorganism in the biological sample.

In certain embodiments, the method further comprises:
providing spectroscopic data within a tenth spectral region from 1000-400 cm⁻¹ as an input into at least one trained machine learning model associated with the tenth spectral region, and responsive to providing the spectroscopic data within the tenth spectral region, identifying at least one microorganism in the biological sample.

In certain embodiments, the method further comprises:
providing spectroscopic data within an eleventh spectral region from 1500-1200 cm⁻¹ as an input into at least one trained machine learning model associated with the eleventh spectral region, and responsive to providing the spectroscopic data within the eleventh spectral region, identifying at least one microorganism in the biological sample.

In a third aspect of the present invention, there is provided apparatus comprising at least one memory for storing spectroscopic data and at least one processor, communicatively coupled to the memory, and configured to perform the steps of the method of the first aspect of the invention or the further aspect of the invention.

In a fourth aspect of the present invention, there is provided a system comprising at least one memory for storing spectroscopic data and at least one processor, communicatively coupled to the memory, and configured to perform the steps of the method of the first aspect of the invention or the further aspect of the invention.

In certain embodiments, the at least one memory is distally remote from the at least one processor. In certain embodiments, the at least one memory is a cloud-based memory.

In a fifth aspect of the present invention, there is provided a computer program comprising instructions which, when the program is executed by a computer, cause the computer to carry out the steps of the method of the first aspect of the invention or the further aspect of the invention. In certain embodiments, the computer program is a non-transitory computer program.

In a sixth aspect of the present invention, there is provided a computer-readable data carrier having stored thereon the computer program of the fifth aspect of the invention. In certain embodiments, the computer readable data carrier is a non-transitory computer readable data carrier.

Certain embodiments of the present invention may have utility in the identification of individual microorganisms and also individual organisms present in a mixture of bacterial and fungal species e.g. combinations such as bacterium/bacterium, bacterium/fungus, fungus/fungus, bacterium/virus, fungus/virus. Aptly, the combination of microorganisms is not limited to binary combinations and includes both planktonic and biofilm phenotypes. In certain embodiments, the invention also provides information as to whether to organism is resistant or sensitive to antimicrobial agents.

In certain embodiments, for the prediction of microorganisms, c-SVM, nu-SVM, stochastic gradient descent SVM and Logistic regression may be used, together with PCA.

In certain embodiments, for the prediction of and analysis of spectral maps ANN (Artificial Neural Networks) and CNN (Convolutional neural networks) may be used.

In certain embodiments, in IR spectroscopy, different compiled IR spectra may be collected and analysed. In the case of pathogenic bacteria affecting humans, a gram stain may be carried out and analysed with IR, adding an extra analysis for disease prediction validation.

In certain embodiments, IR e.g. FTIR (fourier transform infrared) may be used in conjunction with ATR (Attenuated Total Reflectance), sampling accessory of any kind including fibreoptic probe and micro-spectroscopy or any related sampling accessory, such as, grazing angle, Photo-Acoustic sampling cell and others.

Certain embodiments provide near-instantaneous Mid-Infrared spectroscopic screening measurements using nasopharyngeal swab samples, compatible with current sample collection methodology, with no additional single-use plastic consumables, reagents or manufacturing requirements, using Cloud-based FTIR spectrometers, optimised for the test.

Certain embodiments of the present invention may provide one or more of the following technical advantages:
For the detection of microorganisms, the use of gram staining and infrared spectroscopy in combination may help in the rapid differentiation of pathogenic microorganisms giving doctors a quick treatment decision window and thus improve the quality of life of patients.
Several supervised and/or unsupervised methods can be used as validation and comparison that have proven to have excellent accurate results.
Raman spectroscopy maps together with neural networks may provide an extra validation of the microorganism to be predicted and detected.
Evaluation of different microorganisms taken from the same or different samples.
The testing relies on any spectrometer, which may include a benchtop and/or portable spectrometer. This will be cleaned with a standard wipe to clean the crystal, or slide, or any window which may be used as a sampling accessory between measurements, removing the need for cartridges or specialized reagents, greatly simplifying the supply chain. The testing is thus very cost effective.
No production schedules or stockpiling of components would be required. Deployment would simply involve placement of the spectrometer at a standard testing station.
The Cloud-based results would be immediately available from a central database, allowing real-time mapping of the infection.
the absence of additional single-use plastics.
the portability of the instrument means that it can be relocated very easily.
the Cloud-based processing allows real-time tracking of the results, made available electronically.
The system's use of existing sample collection techniques means it can be quickly integrated.

### BRIEF DESCRIPTION OF THE FIGURES

Aspects and embodiments of the present disclosure are described herein, by way of non-limiting example only, with reference to the following drawings in which:

FIG. 1 is a schematic representation of a method according to certain embodiments of the present invention;

FIG. 2 is a flow diagram showing a method of training multiple machine learning modules using the application of pre-processing algorithms according to certain embodiments of the present invention;

FIG. 3 is a spectrogram in which the spectra of the SA (Staphylococcus aureus) organism and the combination of two organisms, KP (Klebsiella pneumoniae) and SA, are compared;

FIG. 4 illustrates Raman spectra of two species of Klebsiella Pneuomoniae;

FIG. 5 illustrates average spectrum from positive and negative samples of Cepheid PCR assay (SARS-COV-2);

FIG. 6 illustrates average spectrum from positive and negative samples of Panther PCR assay (SARS-COV-2);

FIG. 7 is PCA of IR spectra of Cepheid PCR assay samples;

FIG. 8 is PCA of IR spectra of Panther PCR assay samples; and

FIG. 9 shows Negative and positive average spectra of saliva samples;

FIG. 10 illustrates an example neural network;

FIG. 11 illustrates a discrimination analysis and absorbance spectra of certain gram positive bacteria samples in a ‘full wavenumber’ spectral region (4000-400 cm⁻¹);

FIG. 12 illustrates a discrimination analysis and absorbance spectra of certain gram positive bacteria samples in a ‘fingerprint’ spectral region (1800-400 cm⁻¹);

FIG. 13 illustrates a discrimination analysis and absorbance spectra of certain gram positive bacteria samples in a 1800-1710 cm⁻¹ spectral region;

FIG. 14 illustrates a discrimination analysis and absorbance spectra of certain gram positive bacteria samples in a 1590-1290 cm⁻¹ spectral region;

FIG. 15 illustrates a discrimination analysis and absorbance spectra of certain gram positive bacteria samples in an ‘Amide 2’ spectral region (1600-1500 cm⁻¹);

FIG. 16 illustrates a discrimination analysis and absorbance spectra of certain gram negative bacteria samples in a ‘full wavenumber’ spectral region (4000-400 cm⁻¹);

FIG. 17 illustrates a discrimination analysis and absorbance spectra of certain gram negative bacteria samples in a ‘fingerprint’ spectral region (1800-400 cm⁻¹);

FIG. 18 illustrates a discrimination analysis and absorbance spectra of certain gram negative bacteria samples in a 1800-1710 cm⁻¹ spectral region;

FIG. 19 illustrates a discrimination analysis and absorbance spectra of certain gram negative bacteria samples in a 1590-1290 cm⁻¹ spectral region;

FIG. 20 illustrates a discrimination analysis and absorbance spectra of certain gram negative bacteria samples in an ‘Amide 2’ spectral region (1600-1500 cm⁻¹);

FIG. 21 illustrates a discrimination analysis and absorbance spectra of certain urine-bacteria samples in a ‘full wavenumber’ spectral region (4000-400 cm⁻¹);

FIG. 22 illustrates a discrimination analysis and absorbance spectra of certain urine-bacteria samples in a ‘fingerprint’ spectral region (1800-400 cm⁻¹);

FIG. 23 illustrates a discrimination analysis and absorbance spectra of certain urine-bacteria samples in a 1800-1710 cm⁻¹ spectral region;

FIG. 24 illustrates a discrimination analysis and absorbance spectra of certain urine-bacteria samples in a 1590-1290 cm⁻¹ spectral region;

FIG. 25 illustrates a discrimination analysis and absorbance spectra of certain urine-bacteria samples in an ‘Amide 2’ spectral region (1600-1500 cm⁻¹);

FIG. 26 illustrates a discrimination analysis and absorbance spectra for positive and negative saliva samples for SARS-COV-2 virus in a ‘full wavenumber’ spectral region (4000-400 cm⁻¹);

FIG. 27 illustrates a discrimination analysis and absorbance spectra for positive and negative saliva samples for SARS-COV-2 virus in a ‘high wavenumber’ spectral region (4000-2500 cm⁻¹);

FIG. 28 illustrates a discrimination analysis and absorbance spectra for positive and negative saliva samples for SARS-COV-2 virus in a ‘fingerprint’ spectral region (1800-400 cm⁻¹);

FIG. 29 illustrates a discrimination analysis and absorbance spectra for positive and negative saliva samples for SARS-COV-2 virus in an ‘Amides’ spectral region (1800-1300 cm⁻¹);

FIG. 30 illustrates a discrimination analysis and absorbance spectra for positive and negative nasopharyngeal samples for SARS-COV-2 virus in a ‘full wavenumber’ spectral region (4000-400 cm⁻¹);

FIG. 31 illustrates a discrimination analysis and absorbance spectra for positive and negative nasopharyngeal samples for SARS-COV-2 virus in a ‘high wavenumber’ spectral region (4000-2500 cm⁻¹);

FIG. 32 illustrates a discrimination analysis and absorbance spectra for positive and negative nasopharyngeal samples for SARS-COV-2 virus in a ‘fingerprint’ spectral region (1800-400 cm⁻¹);

FIG. 33 illustrates a discrimination analysis and absorbance spectra for positive and negative nasopharyngeal samples for SARS-COV-2 virus in an ‘Amides’ spectral region (1800-1300 cm⁻¹); and

FIG. 34 illustrates a spectrometer and connected computing device.

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e., to at least one) of the grammatical object of the article. By way of example, “an element” means one element or more than one element.

In the context of this specification, the term “about,” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

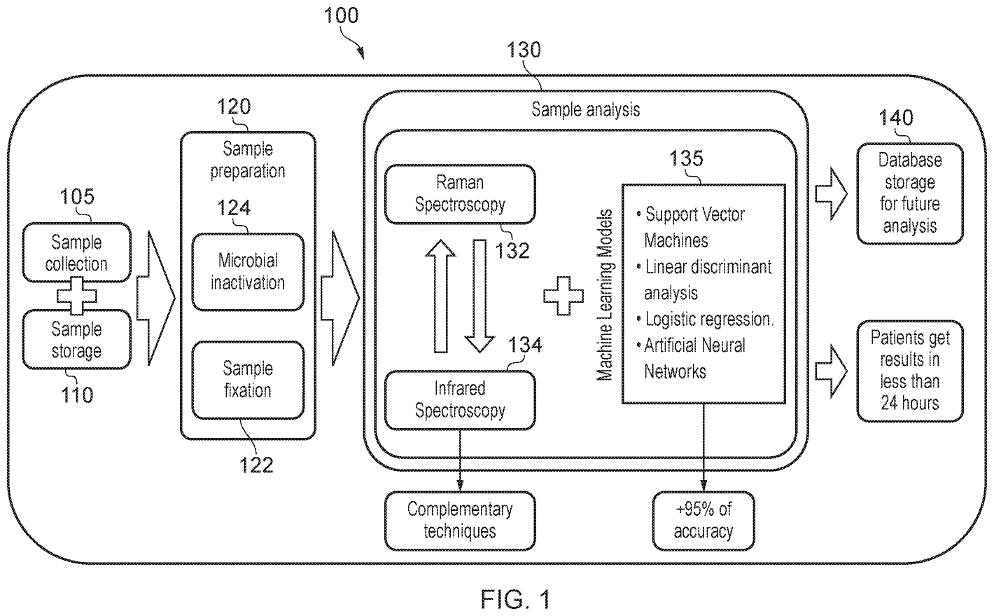

FIG. 1 helps to illustrate a method 100 according to certain embodiments of the present invention. Aptly the method 100 may be a method of identifying at least one microorganism in a biological sample. Whilst the term microorganism is often referred to herein it will be appreciated that other terms may be used interchangeably such as microbe, bacterium and the like. A first step of the method 100 involves a sample collection step 105 and an optional sample storage step 110.

In the sample collection step 105 the samples may be available or obtained for example as:
i. Direct patient samples, these will be samples of blood or body fluid containing pathogenic micro-organisms. The samples fall into 2 groups:
   a) Normally sterile sites where the presence of any microorganism is likely to be significant.
   b) Non-sterile sites where the sample will contain a mixture of pathogenic (harmful) organisms along with the organisms normally present at this site (commensals). or
ii. Cultured samples. In this case the samples collected as above are processed in a diagnostic laboratory to increase the number of bacteria present. This can occur in 2 ways:
   a) The sample is paced onto a solid culture medium and incubated at 37° C. to encourage growth of the microorganism. Samples from these culture media can be placed directly into our system using either a swab or a loop of sterile plastic.
   b) The sample is placed into a liquid culture medium and incubated at 37° C. to encourage growth of the microorganism. Samples from these culture media can be placed directly into our system using either a swab or a loop of sterile plastic.

In the sample storage step 110, the samples may optionally be placed either directly into sterile containers or onto a swab which will contain a transport medium to prevent degradation of the sample on the way to the laboratory. Alternative sample collection and storage methodologies may be employed according to certain embodiments of the present invention as will be appreciated by a person skilled in the art.

Turning back to FIG. 1, following the sample collection step 105 and the sample storage step 110 is a sample preparation step 120. The preparation of the sample depends on the nature of the sample. If the sample is a previously isolated microorganism, but whose nature is unknown, collecting a bacterial colony that was cultivated during the night at 37° C. with an inoculation loop may be used. The bacterial colony sample taken with the inoculation handle is suspended in 15 ml of deionised water. With a micropipette, the microbial suspension is resuspended and 200 ul are taken to be placed on a CaF₂ (Calcium Fluoride) slide. Using CaF₂ slides enables data to be obtained in transmission/absorbance modes, which provides excellent spectral data with good signal to noise ratio. Furthermore, CaF₂ slides do not interfere with the analytes/specimen, provide low refraction index, as well as reduced noise. This is an advantageous way of obtaining data from biofluids, as the entire sample can be analysed in transmission/absorbance modes. This allows information form the entire sample (bulk and surface) to be obtained (i.e., the data obtained is not limited to spectral data from the surface of a sample as is the case with certain other techniques). The slide with the liquid microbial sample is placed on a hot plate at 60° C. to fix the liquid sample in a sample fixation step 122.

In the case of a biofluid sample obtained directly from a patient; the sample may be obtained either from urine, saliva, blood or/and directly from an exudate, or from a swab. The sample is smeared on the CaF₂ slide (or any suitable substrate (such as an ATR crystal) which may be used as a window or/and a slide) and placed on a hot plate at 40-80° C. to fix the sample in a sample fixation step 122 and/or reduce the infectivity of the sample in a microbial inactivation step 124. In the microbial inactivation step 124, heating the sample to 65° C. for 2 mins may result in a 6 log reduction of most pathogens. Some vegetative bacteria and spores may not be inactivated by heat fixing so even after heat fixing the slides should be handled with care. More details on microbial inactivation can be found at the following links:
https://www.who.int/water_sanitation_health/dwg/Boiling_water_01_15.pdf
https://www.ncbi.nlm.nih.gov/pmc/articles/PMC5742775/

Alternative sample preparation methodologies may be employed according to certain embodiments of the present invention as will be appreciated by a person skilled in the art. Aptly, Raman spectroscopy can be used to analyse aqueous samples, which does not require a sample fixation step. It is possible to analyse aqueous samples directly spread onto a CaF₂ slide, or any suitable substrate which may be used as a window or/and a slide.

FIG. 1 also illustrates a sample analysis step 130. In certain embodiments, Raman spectroscopy and/or Infrared spectroscopy are used. Certain embodiments of the present invention involve the use of infrared and/or Raman spectroscopic techniques. The sample analysis step 130 illustrates the Raman spectroscopy technique 132 and the infrared spectroscopy technique 134 used to obtain spectroscopic data from the biological sample. Aptly, to obtain the Raman and/or IR spectroscopic data, a 480 nm and/or 532 nm and/or 633 nm and/or 780 nm and/or 1064 nm laser can be used to excite the sample being analysed. The infrared and Raman spectroscopic techniques are discussed in further detail below.

In recent years, both Infrared and Raman spectroscopic techniques have emerged as powerful tools for chemical analysis because of their ability to provide detailed information on the spatial distribution of chemical composition at the molecular level. In applications requiring qualitative and quantitative analysis, these techniques have the potential to identify chemical components via fingerprinting analysis of their vibrational spectrum.

Both these techniques fall within the classification of vibrational spectroscopy, which is a powerful light scattering/absorption technique used to investigate the internal structure of molecules and crystals. As the technique is specific to the chemical bonds and molecular structures, it is commonly used in chemistry and has been considerably covered by scientists and research groups from different disciplines. Certain embodiments of the present invention are based on the spectra being highly detailed allowing subtle differences in biochemistry to be identified. In principle, any physiological change or pathological process that results in changes to the native biochemistry would therefore lead to changes in the IR and Raman spectra. For example, IR and Raman spectra of different biological samples, such as tissues, cells and cell lines can be analysed giving rise to different spectra. These techniques can determine the dissimilarities of different types of cells at molecular level. The techniques can identify the functional groups and chemical bonds that are present in the biological tissues or/and cells. Therefore, it is possible not only to evaluate the structure of the proteins, lipids, carbohydrates, and nucleic acids that are present in a biological molecule, but also the changes that are taking place in their chemical structure due to the disease process. Hence, making it possible to monitor the progression of the disease process allowing to predict the chemical pathway of the progression of a disease process.

Infrared spectroscopy mainly deals with the infrared region of the electromagnetic spectrum and most commonly focuses on absorption spectroscopy (when the frequency of the IR is the same as the vibrational frequency of a bond, absorption occurs). When a material is exposed to infrared radiation, absorbed radiation usually excites molecules into a higher vibrational state. The wavelengths that are absorbed by the sample are characteristic of its molecular structure. However, like other spectroscopic techniques, this technique can also identify molecular structure and investigate sample composition and the spectral bands are indicative of molecular components and structures.

According to certain embodiments of the present invention, the infrared spectroscopy technique is an FTIR (Fourier-transform infrared spectroscopy) technique. Aptly, the infrared spectroscopy technique may be an FTIR coupled with an ATR (Attenuated Total Reflectance spectroscopy) technique. This may be referred to as Attenuated Total Reflectance Fourier transform Infrared spectroscopy (ATR-FTIR). An ATR accessory operates by measuring the changes that occur in an internally reflected IR beam when the beam comes into contact with a sample. An IR beam may be directed onto an optically dense crystal. ATR allows samples to be mounted on different crystals, such as, but not limited to diamond, germanium, silicon, KRS-5 (Thallium Bromide Iodide) crystals. These materials enable the provision of spectral signatures at different depths of the sample, depending on the sample used with the mounting sample accessory. An FTIR spectrometer simultaneously collects high-resolution spectral data over a wide spectral range. An FTIR spectrometer is typically used for measurements in the mid and near IR regions. The IR source for the mid-IR region may be a silicon carbide element heated to about 1200K. Shorter wavelengths of the near-IR (10000-4000 cm⁻¹) may use a tungsten-halogen lamp. For far-IR, a mercury discharge lamp may be used. An FTIR spectrometer may also comprise a detector and a beam splitter. In certain embodiments, the FTIR spectrometer is used with an accessory e.g. an attenuated total reflectance, which is capable of measuring surface properties of solid or thin film samples rather than their bulk properties. Attenuated total reflectance (ATR) is a contact sampling method that is quick, non-destructive, and requires no to minimal sample preparation. A variety of ATR accessory designs exist and are available from a number of manufacturers of IR accessories. ATR is a sampling technique usable in conjunction with Infrared (IR) spectroscopy, where the ATR accessory may be (i) in the sample compartment of the IR spectrometer or (ii) on top of the spectrometer in spectrometers that have a built in ATR accessory or (iii) mounted on a microscope objective (termed as ATR objective) and microscope attached to the spectrometer. ATR may use diamond, germanium, silicon, KRS5 (Thallium Bromide Iodide) crystals or the like for mounting the sample. Using ATR-FTIR and these materials enables the provision of spectral signatures at different depths of the sample, depending on the sample used and the mounting sample accessory. Additionally, ATR-FTIR spectrometers may be portable. This allows detection of micro-organisms to take place at any appropriate location and reduces the need for samples to be sent to a laboratory for analysis. When an IR beam travels from a medium of high refractive index (e.g. diamond, germanium, zinc selenide crystal etc.) to a medium of low refractive index (e.g. sample), some amount of the light is reflected back into the low refractive index medium. At a particular angle of incidence, almost all of the light waves are reflected back. This phenomenon is called total internal reflection. In this condition, some amount of the light energy escapes the crystal and extends a small distance (0.1-5 μm) beyond the surface in the form of waves. The intensity of the reflected light reduces at this point. This phenomenon is called attenuated total reflectance. When the sample (liquid or solid) is applied on the crystal some amount of the IR radiation penetrating beyond the crystal is absorbed by the sample. This absorbance is translated into the IR spectrum of the sample. A background spectrum may be obtained by using a clean neat crystal.

IR spectroscopy is a frequency vibrational spectroscopy technique and is a useful tool available to scientists when it comes to solving a problem involving having to discover the molecular structure, molecular behaviour and/or the identification of unknown organic chemical substances and mixtures. When the material under investigation is subjected to an IR source, it will absorb the radiation emitted (typically infrared radiation) and the successful absorption will display the uniqueness or “fingerprint” of the material under investigation.

The infrared spectrum is recorded by passing a beam of infrared light through the sample and recording the changes at the energy level of the photons because of interactions with the sample. This can be done with a monochromatic beam, which changes in wavelength over time. However, using a Fourier Transform (FT) instrument makes it possible to measure all wavelengths at once. The aim is to measure the quality and quantity of transmittance or absorbance of each different wavelength by a sample that can produce transmittance or absorbance spectrum. An infrared radiation source within the mid-IR range (4000-400 cm⁻¹) is used. When the sample is exposed to the IR beam, different vibrational modes cause change in the electronic conformation of the dipole moment, which is detected by a detector.

Depending upon the nature of sample(s), different sampling accessories may be used for surface or bulk analysis of the sample.

Both the FTIR and Raman spectroscopic techniques are complimentary techniques to each other providing chemical structural properties of the biological molecules. Both the techniques can be used for rapid detection and identification of microbes and antibiotic sensitivity work using a pure sample from a culture plate. For example, certain spectral characteristics may be visible and identifiable only via one technique or the other. Hence, having both techniques to analyse samples provides complete fingerprinting of the sample that is being analysed, giving an enhanced confidence level.

IR spectroscopy works on the fact that chemical bonds or groups of bonds vibrate at characteristic frequencies. A molecule that is exposed to infrared rays absorbs infrared energy at frequencies that are characteristic to that molecule. This technique works almost exclusively on samples with covalent bonds. This is different from a Raman effect, which mainly deals with polarizability of chemical bonds. Therefore, in simple terms, IR spectroscopy detects change in the dipole moment of the molecules, whereas Raman spectroscopy analyses change in the polarisation of molecule. For a molecule to be infrared active it should have a dipole moment.

Raman Spectroscopy is a vibrational spectroscopic technique that is used to optically probe the molecular changes associated with diseased tissues. The technique is based on different types of scattering, of monochromatic light, usually from a laser in the visible, near infrared, or near ultraviolet range, hence different types of lasers can be employed. Light from the illuminated spot is collected with a lens and sent through a monochromator.

Raman spectroscopy detects the change in the polarisation of a molecule. Raman spectra are a plot of scattered intensity as a function of the energy difference between the incident and scattered photons and are obtained by pointing a monochromatic laser beam at a sample. When light strikes a molecule, most of the light is scattered at the same frequency as the incident light (elastic scattering). Only a small fraction is scattered at a different wavelength (inelastic or Raman scattering) due to light energy changing the vibrational state of molecule. The loss (or gain) in the photon energies corresponds to the difference in the final and initial vibrational energy levels of the molecules participating in the interaction. The resultant spectra are characterized by shifts in wave numbers (inverse of wavelength in cm⁻¹) from the incident frequency. The frequency difference between incident and Raman scattered light is termed the Raman shift, which is unique for individual molecules and is measured by the machines detector and is represented as 1/cm. Raman peaks are spectrally narrow, and in many cases can be associated with the vibration of a particular chemical bond (or a single functional group) in the molecule. The vibrations are molecular bond specific allowing a ‘biochemical fingerprint’ to be constructed of the material.

For microsamples, an integrated microscope with a spectrometer may be used to visually locate the sample. The 10×, 50×, and 100× objectives may also be employed depending upon the nature of the sample. Once the specimen is located, the spectra data acquisition parameters are optimised. For example, laser power on the sample, exposure time, number of scans, mapping area of the image, aperture size, spectral and spatial resolution etc. The advantage of using a microscope is that the combination of microscopy with Raman and FTIR allows all of the different organisms in a sample, which may have different shapes and staining characteristics to be identified. Additionally, using a microscope attached to an Infrared/Raman spectrometer allows the creation of spectral maps of the specimen, which enables areas where different microbes, or mixtures of microbes are present to be located. For example, when a microscopic slide is examined, it may be seen that there is an abundance of one species in one region but different microbes in another region. In addition, a microscope attached to an FTIR or Raman spectrometer also enables the analysis of tissue biopsies with the advantage of obtaining a chemical structural map of a tissue. Furthermore, while analysing biofluids on a CaF₂ slide, when the sample is dried it can sometimes form crystals at the edges (this is especially the case when urine samples are being analysed). However, the mapping capability of the FTIR/Raman spectrometer with attached microscope nevertheless allows the analysis of chemical variations among the samples/specimens.

According to certain embodiments of the present invention, Raman Spectroscopy may be performed by using either Thermo Fisher Scientific Raman DxR, or Thermo Fisher Scientific Raman DxRxi, or any equivalent spectrometer to those mentioned above, independently of the make and model. FTIR spectroscopy may be performed using either Thermo Fisher Scientific Nicolet iN10, or Thermo Fisher Scientific Nicolet iS50, or Thermo Fisher Scientific Summit Pro, or any equivalent spectrometer to those mentioned above, independently of make and model.

Biological samples may possess fluorescence, this is a natural phenomenon as some molecules may absorb high amounts of energy and gradually release their energy affecting the measurement in the spectrometer. For these cases, 30 s to 1.5 min of photobleaching may be used.

Clinical samples containing different type of bacteria in a complex mixture can be analysed spectroscopically and instead of just reporting very basic information from the gram film could rapid detection, identification, and sensitivity data may be provided.

In some embodiments, in the sample analysis step 130 one or more trained machine learning models 135 are used. It will be appreciated that a trained machine learning model is a model stored in memory that has been trained via providing a machine learning module with input training data. It will be appreciated that a machine learning module is a module comprising at least one processor that executes one or more machine learning algorithms stored in at least one memory within the module and/or remote from the module. Aptly, each machine learning model 135 is obtained via training each of a set of respective machine learning modules. Aptly, each machine learning module and thus each trained machine learning model is a multinomial classifier. In certain embodiments, each machine learning module is one of a linear discriminant analysis based module, a support vector machine based module, a logistic regression based module, a K nearest neighbours based module, a random forest based module, a partial least squares module, an artificial neural network based module or a convolutional neural network based module. Corresponding trained machine learning models may thereby be provided. One or more of the models may be able to identify microorganism(s) within a biological sample with greater than 95% accuracy and/or facilitate provision of results to patients in less than 24 hours. This is the case even if the sample is a direct biofluid sample taken from a patient which typically have much more background noise that pure culture specimens. The person skilled in the art will appreciate that any number of trained machine learning models may be used in certain embodiments of the present invention.

According to certain embodiments of the present invention, the machine learning models are stored in at least one memory. The memory may be a local memory within the Raman and/or Infrared spectrometer. Alternatively, the memory may be a memory within a computing device connected to the Raman and/or Infrared spectrometer via a Local Area Network. Alternatively, the memory may be a memory connected to the Raman and/or Infrared spectrometer via a Wide Area Network (e.g. the Internet).

A further step 140 of the method involves providing the spectroscopic data, in the form of spectra files (e.g. .csv format extension or any other spectrum file extension such as .spa), to a database for future analysis. The database may also be stored in at least one memory. The memory may be the same memory which stores the trained machine learning models or may be a different memory. For example, the memory may be a local memory within the Raman and/or Infrared spectrometer. Alternatively, the memory may be a memory within a computing device connected to the Raman and/or Infrared spectrometer via a Local Area Network. Alternatively, the memory may be a memory connected to the Raman and/or Infrared spectrometer via a Wide Area Network (e.g. the Internet). Alternatively, the memory may be a cloud-based memory. According to certain embodiments of the present invention, the model stored within memory may obtain the spectroscopic data from the database stored in cloud-based memory. A folder storing spectra files in the computing device where the models are stored may have direct access to the cloud allowing it to synchronize as the spectra files are updated.

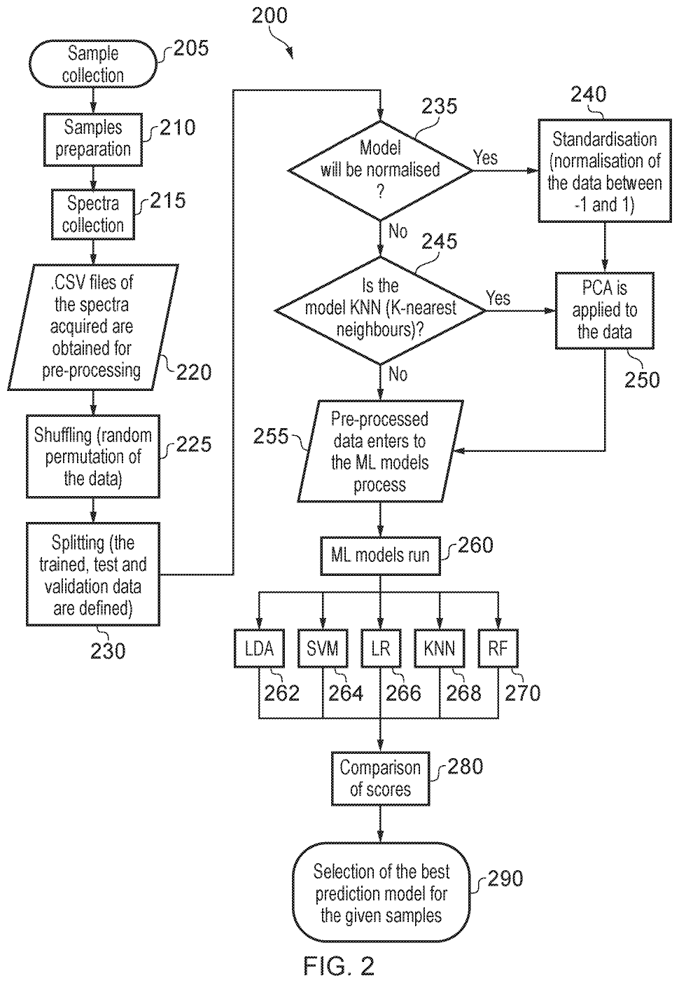

FIG. 2 helps to illustrate a method 200 of training multiple machine learning modules using the application of pre-processing algorithms. The pre-processing algorithms are applied to training spectroscopic data prior to inputting the training data into untrained or part trained machine learning modules.

A first step of the method 200 is a sample collection step 205. The sample collection step 205 is a step similar to the sample collection step 105 as illustrated and described with respect to FIG. 1. Then, a sample preparation step 210 is performed. The sample preparation step 210 is a step similar to the sample preparation step 110 illustrated and described with respect to FIG. 1. Following collection and preparation of the appropriate biological samples, at a spectra collection step 215, training spectroscopic data 220 is obtained via an infrared spectroscopy technique or a Raman spectroscopy technique. For example, this involves placing a slide that has a biological sample fixed to its surface at a target location of a Raman or Infrared spectrometer where electromagnetic radiation is directed at the sample. The electromagnetic radiation interacts with the sample and the Raman or Infrared spectrometer then collects transmitted or reflected or non-absorbed radiation via an objective lens and/or a fibre optic probe. The collected radiation is then passed through a series of optical elements (mirrors, gratings etc.) onto a photodetector such as a charge coupled device (CCD), photomultiplier tube or the like. The detector readings are then processed electronically into spectroscopic data 220. The training spectroscopic data 220 is representative of a spectral response of one or more microorganisms present in the biological sample.

The training spectroscopic data 220 may be stored in one or more databases. Aptly, the data stored is from spectra of known organisms. The trained machine learning models may use these spectra to provide the basis of comparison for the unknown organisms which will be identified by their closeness of match to known spectra held in the database. The data may be presented to the end user as a series of identifications, organism names, with percentage match to known organisms. The database may be a folder organised into different subfolders with spectra files obtained from the spectrometer and stored in a specific spectral format (Name.SPA and Name.CSV) files will be saved from each sample analysed. The value is in the range of spectra and provides the ability to compare, via the trained models, known with unknown spectra to provide identification and antimicrobial sensitivity. Aptly, a searchable spectral library of known microbes is created and used for identification of unknown microbes. An example of a folder containing some spectra is illustrated in Table 1 below.

Aptly, the database comprises data in .spa and .csv format (or any other spectrum file extension format) in a folder located on a computer's hard disk where the trained machine learning models may be executed. The database is created as different microorganisms are collected and identified by spectroscopic methods. That is, strains of micro-organisms from National Type Collections are used initially as standard strains. Subsequent strains of bacteria are identified using standard clinical laboratory protocols.

The database may be used as the main resource for extracting the files. The code calls the site where the .csv files are stored and uses them as raw material to run the machine learning models.

In certain embodiments, the input training spectroscopic data 220 contains Raman and/or Infrared Spectra where the following regions are studied:
Spectral Region (between 3200 to 400 cm⁻¹ in Raman spectroscopy, and between 4000 to 400 cm⁻¹ in the case of Infrared spectroscopy). In some cases, Raman analysis may be done down to 50 cm⁻¹ region.
Fingerprint Region (between 1800 to 400 cm⁻¹ for both Raman (may be down to 50 cm⁻¹ if required) and Infrared Spectroscopy).
High Wavelength Region (between 3200 to 2700 cm⁻¹) in Raman Spectroscopy, and 4000 to 2700 cm⁻¹ in the case of Infrared Spectroscopy).

Aptly, each region has its own set of models tailored towards the input training data. The feature values in the spectrogram measurement data are the amplitudes at each wavelength in the region measured. The input data is transformed into a feature matrix X of size n×m with n samples or instances (spectrograms) and m features (wavelengths).

To train or retrain the models an additional vector y containing the encoded class label (e.g. bacteria strain) can be appended row-wise to the matrix X resulting in a matrix of size n×m+1. Encoding is numerical—the classes in vector y are mapped to numbers in the range [1,c] where c is the number of classes (e.g. bacteria strains). “C” are the classes given previously to the identification of the strains. An example matrix is shown in Table 2 below.

The matrix “X” (0, 0.92-0.80 etc.) shown in Table 2 below has “m” columns (wavenumber 4000, 3999 etc.), “n” rows or instances (microorganism 1-1 etc.) and an Appended Y column with microbial strain labels. In the example from below there are 3 “C” groups (SA, KP, SE). This value is given previously, therefore is not an arbitrary value.

TABLE 2

| | 4000 | 3999 | m + 1 . . . | 400 | Appended Y |
|---|---|---|---|---|---|
| Microorganism 1-1 | 0 | 0.92 | . . . | −0.80 | SA |
| Microorganism 1-2 | 0 | 0.93 | . . . | −0.85 | SA |
| Microorganism 1-3 | 0 | 0.94 | . . . | −0.75 | KP |
| Microorganism 1-4 | 0 | 0.95 | . . . | −0.77 | KP |
| Microorganism 1-5 | 0 | 0.96 | . . . | −0.76 | SE |
| Microorganism 1-6 | 0 | 0.95 | . . . | −0.74 | SE |
| . . . | . . . | . . . | . . . | . . . | . . . |
| Microorganism n + 1 | 0 | 0.98 | . . . | −0.71 | Microbial |
| | | | | | strain |

In certain embodiments, the feature vector matrix is processed for training purposes. Aptly, the method comprises several processing steps—shuffling, splitting, standardisation and PCA. Aptly, the input spectroscopic data 220 is pre-processed using a sequence of algorithms. Aptly, for training the machine learning models the following sequence can be used as illustrated in FIG. 2.

Following the spectra collection step 215 and storing spectroscopic data 220 in a database. A first pre-processing step is a shuffling step 225. The shuffling step 225 permutes the data randomly. The shuffling may for example be performed using the numpy.random.permutation( ) class from the numpy package. Other shuffling methods may be used as will be appreciated by a person skilled in the art. The label np.random.permutation( ) is just an abbreviate and has source of the following code, of python language.

| | |
|---|---|
| 3280 | def permutation(self, object x): |
| ... | |
| 3307 | if isinstance(x, (int, np.integer)): |
| 3308 | arr = np.arange(x) |
| 3309 | else: |
| 3310 | arr = np.array(x) |
| 3311 | self.shuffle(arr) |
| 3312 | return arr |

Shuffling is applied on the matrix containing the features and class label joined hence the dataset has size n×m+1. Shuffling the spectroscopic data 220 helps the trained models to constantly predict with the same accuracy, allowing the model to have different matrix arrangements to evaluate, and therefore not relying on always the same input. This may give more quality to the data. The following code is a simple example illustrating what happens when shuffling. The input matrix (numbers 1, 4, 9, 12 and 15) always has that same value when running this code, the output will be the same number but shuffled differently.

>>> np.random.permutation([1, 4, 9, 12, 15])
array([15, 1, 9, 4, 12])

After the spectroscopic data 220 has been shuffled in the shuffling step 225, a second pre-processing step is a splitting step 230. The splitting step 230 involves dividing the spectroscopic data into train, validation and test datasets. Splitting the data into these three datasets allows the models to identify the labelled spectra and the unlabelled spectra. The labelled spectra are used for training and validation of the machine learning models. The unlabelled spectra are used for testing the trained machine learning models. These parameters (labelled or unlabelled) are used only by delimiting them in the coding, the splitting of the data discriminates between whether a spectrum is labelled or not. Table 3 below illustrates an example feature matrix that has had a splitting step 230 applied. The coloured rows are the rows including spectroscopic data from known microorganisms (SA, SE) that has been used for the training dataset and the validation dataset. These rows are used to train the machine learning models to identify these microorganisms. The non-coloured rows are the rows of the test dataset. These rows are used to test the prediction capability of the trained machine learning models. For each model a train dataset, test dataset, and validation may be used. The train dataset defines the labels to be identified by the model so the model knows the microorganism associated with the spectroscopic data. Test datasets is data that is within the same matrix to be analysed. This data is not labelled and is assessed by the model previously given the train datasets. During the splitting step 230, both the train datasets and test datasets obtained by using the function sklearn.model_selection.train_test_split, the size of the train dataset is defined as a real value between 0 and 1 (e.g. between 0.7 to 0.8). The size of the test dataset is likewise defined as a real value and is the complement of the train dataset size (e.g. between 0.2 to 0.3). Other splitting methods may be used as will be appreciated by a person skilled in the art. On the other hand, validation is a step that may be executed to evaluate if the model used is overfitting. The function sklearn.model_selection.cross_val_score may be used which takes the test dataset to validate the model efficiency by randomizing the train dataset and the test dataset into different folds, (e.g. 5 to 10 folds). The average validation score is then obtained from each number of k folds provided. The person skilled in the art will appreciate that other validation techniques may also be used.

TABLE 3

| | 4000 | 3999 | m + 1 . . . | 400 | Appended Y |
|---|---|---|---|---|---|
| Microorganism 1-1 | 0 | 0.92 | . . . | −0.80 | SA |
| Microorganism 1-2 | 0 | 0.93 | . . . | −0.85 | SA |
| Microorganism 1-3 | 0 | 0.94 | . . . | −0.75 | |
| Microorganism 1-4 | 0 | 0.95 | . . . | −0.77 | |
| Microorganism 1-5 | 0 | 0.96 | . . . | −0.76 | SE |
| Microorganism 1-6 | 0 | 0.95 | . . . | −0.74 | SE |
| . . . | . . . | . . . | . . . | . . . | . . . |
| Microorganism n + 1 | 0 | 0.98 | . . . | −0.71 | Microbial |
| | | | . . . | | strain |

Following the splitting step 230 there is an option to normalise the spectroscopic data in a standardisation step. If a user and/or pre-programmed algorithm decides to normalise the data at a first decision step 235, then the spectroscopic data 220 enters a standardisation step 240. If a user and/or pre-programmed algorithm decides not to normalise the data at the first decision step 235, then the spectroscopic data 220 continues to a second decision step 245 discussed below. The standardisation step 240 scales the values in the feature matrix X into a defined range.

Optionally, the standardisation method chosen may be the MaxAbsScaler( ) method from sklearn.preprocessing library which scales each feature individually to the [−1, 1] range. Other normlisation methods may be used as will be appreciated by a person skilled in the art. MaxAbsScaler( ) is the abbreviation of the following code that will work in python programming to scale between −1 and 1:

def maxabs_scale(X, axis=0, copy=True):
″″″Scale each feature to the [−1, 1] range without breaking the sparsity.
X = check_array(X, accept_sparse=(′csr′, ′csc′), copy=False,
ensure_2d=False, dtype=FLOAT_DTYPES)
original_ndim =
X.ndim
if original_ndim == 1:
X = X.reshape(X.shape[0], 1)
s = MaxAbsScaler(copy=copy)
if axis == 0:
X = s.fit_transform(X)
else:
X = s.fit_transform(X.T).T
if original_ndim == 1:
X = X.ravel( )
return x

The code MaxAbsScaler( ) scans each feature (wavenumbers) on the matrix and automatically identifies the maximum value and the minimum converting it to a scale between −1 to 1. This step is also known as normalisation. This step is performed separately to allow a comparison of the performance of the algorithm to be made when scaling/normalisation is or is not applied. The purpose is to compare efficiencies between unscaled and scaled models.

Following the standardisation step 240 or following a decision not to normalise at decision step 235, a decision is made by the user and/or by a pre-programmed algorithm as to whether to apply Principal Components Analysis (PCA) to the spectroscopic data at a second decision step 245. This involves checking whether machine learning module is a K nearest neighbours based module. If the module to be trained is a K nearest neighbours based module, PCA will be applied to the spectroscopic data at PCA step 250. If the module to be trained is not a K nearest neighbours based module then the method may proceed to the training step 255. The PCA step 250 is thus an optional step. Principal Components Analysis is a linear dimensionality reduction technique based on Singular Value Decomposition of the centred data to project it to a lower dimensional space with a few components on which the data shows the highest variability. The class sklearn.decomposition.PCA can be imported from sklearn package with the number of principal components set up to 10 and other parameters left at default values. Other PCA packages may also be used as will be appreciated by a person skilled in the art. The PCA step 250 transforms the spectroscopic data from a high-dimensional feature space into a low-dimensional feature space without losing meaningful properties of the original data.

Utilising PCA helps to reduce the dimensions of the matrix instead of working with a reduced matrix size. In other words, the PCA step 250 is a method of resetting the coordinate axes (PCA loadings) in the multivariate space so as to quantitatively take features of the point group distribution of the vibrational spectrum in such a multivariate space, and classifying the samples on the basis of position information (PCA score) of the vibrational spectrum in the space expanded by the PCA loadings.

Table 4 below illustrates how the features may be reduced to only two columns. Thus, instead of using 4000 features, the PCA step 250 may reduce this number to only 2 new features. The mathematics behind PCA step 250 is as follows.

First, a mean from each column k (feature) is obtained, where X is the value in the matrix, and N the amount of samples, using the equation below.

$${\overline{X}}_{k} = \frac{{\sum }_{i = 1}^{N} {X}_{i}}{N}$$

Then, the standard deviation of each column is obtained using the following equation, where i is the row of the matrix and k is the column of the matrix.

$${σ}_{k} = {[ \frac{{\sum }_{i = 1}^{N} {( {x}_{ik} - {\overline{x}}_{k} )}^{2}}{( N - 1 )} ]}^{1 / 2}$$

Then, the covariance matrix (Σ) is obtained. Σ is a measure how much each column of data varies from the mean with respect to each other. Where x is the mean divided by the standard deviation, xi is the individual data divided by the standard deviation. Y constants represent the other data in the matrix to be compared within the matrix column (kₙ₊₁)

$$cov ( x , y ) = \frac{{\sum }_{i = 1} ( {x}_{i} - \overline{x} ) ( {y}_{i} - \overline{y} )}{( N - 1 )}$$

A linear transformation T of Matrix E (m×n) which contains different vectors (v1, v2, . . . , vn), will then generate new vectors, b1, b2 . . . bn. Therefore:

Tv₁=b₁

Tv₂=b₂

Tvₙ=bₙ

The new vector change in length but not direction, and are now called eigenvectors, being the eigenvalue the scalar which represents the multiple eigenvectors.

Tvᵢ=λvᵢ

The initial matrix Σ will be multiplied by the new matrix (V) that contains the new eigenvectors (the columns of V are the same number of rows of matrix X), being equal to the eigenvalues matrix (L) times the matrix V.

ΣV=LV

Which is translated to:

$${[ \begin{matrix}Var ( x ) & Cov ( x , y ) \\ Cov ( y , x ) & Var ( y )\end{matrix} ] [ \begin{matrix}{v}_{1} \\ {v}_{2}\end{matrix} ]}^{′} = [ \begin{matrix}{λ}_{1} \\ {λ}_{2}\end{matrix} ] [ \begin{matrix}{v}_{1} \\ {v}_{2}\end{matrix} ]$$

TABLE 4

| | | | PC-1 | PC-2 |
|---|---|---|---|---|
| Scores | | [symbol] | 1 | 2 |
| [symbol] 1 | [symbol] 1 | 1 | 0.3562 | −1.6745 |
| [symbol] 2 | [symbol] 2 | 2 | 0.2985 | −1.6311 |
| [symbol] 3 | [symbol] 3 | 3 | 0.6502 | −1.6680 |
| [symbol] 1 | [symbol] 1 | 4 | −4.5714 | −1.3182 |
| [symbol] 2 | [symbol] 2 | 5 | −4.5953 | −1.4744 |
| [symbol] 3 | [symbol] 3 | 6 | −4.3982 | −1.4693 |
| [symbol] | [symbol] | 7 | 5.3944 | 1.6330 |
| [symbol] | [symbol] | 8 | 5.5281 | 1.5925 |
| [symbol] | [symbol] | 9 | 5.4481 | 1.6274 |
| [symbol] | [symbol] | 10 | 5.2768 | 0.6787 |
| [symbol] | [symbol] | 11 | 5.3063 | 0.6511 |
| [symbol] | [symbol] | 12 | 5. [symbol] | 0.5911 |
| [symbol] | [symbol] | 13 | 4.4 [symbol] 57 | 0.9017 |
| [symbol] | [symbol] | 14 | 4.6643 | 0.8512 |
| [symbol] | [symbol] | 15 | 4.608 [symbol] | 0.8315 |
| [symbol] | [symbol] | 16 | 0.5234 | −0.1272 |
| [symbol] | [symbol] | 17 | 0.5592 | −0.0943 |
| [symbol] | [symbol] | 18 | 0.513 [symbol] | −0.0611 |
| [symbol] | [symbol] | 19 | 3.8491 | 0.3926 |
| [symbol] | [symbol] | 20 | 4.2483 | 0.3283 |
| [symbol] | [symbol] | 21 | 4.2195 | 0.324 [symbol] |
| [symbol] | [symbol] | 22 | 9.4945 | 1.5443 |
| [symbol] | [symbol] | 23 | 9.5679 | 1.5465 |
| [symbol] | [symbol] | 24 | 9.508 [symbol] | 1.5862 |
| [symbol] | [symbol] | 25 | 5.087 [symbol] | 1.5450 |
| [symbol] | [symbol] | 26 | 5.1639 | 1.5482 |
| [symbol] | [symbol] | 27 | 5.4050 | 1.5032 |
| [symbol] | [symbol] | 28 | −3.9591 | −0.2788 |
| [symbol] | [symbol] | 29 | −3.8 [symbol] 09 | −0.3044 |
| [symbol] | [symbol] | 30 | −3.5 [symbol] 05 | −0.2832 |
| [symbol] | [symbol] | 31 | −2.6663 | −0.8485 |
| [symbol] | [symbol] | 32 | −2.3617 | −0.8 [symbol] 67 |
| [symbol] | [symbol] | 33 | −2.4067 | −0.8 [symbol] 79 |

[symbol]

indicates data missing or illegible when filed

In other words, λ and v represent the eigenvalues, and eigenvectors, respectively, of the covariance matrix that will help to define the Principal components to be used. If λ1>λ2 this means λ1 is PC1 and so on. The eigenvectors will then be used for the dimensionality reduction. As noted above, the spectral regions may be identified in three regions, Higher wavenumber 4000 to 2000 cm⁻¹, Fingerprint region (1800 to 100 cm−1) and lower wavenumber (400-50 cm⁻¹). One spectrum represents the whole set of spectral data from the analysis of one sample.

Spectral peak position (wavenumber), their shape (broad, sharp, shoulder etc.) and intensity (absorbance intensity) provide unique chemical structure of the molecule or microorganism that has been analysed. Every microbe/microorganism has a different chemical structure and their unique individual spectrum allow their identification. For example, if there is a specific bacterium present in a clinical sample, it will have a unique spectrum allowing its precise identification within minutes rather than culturing it for a minimum of 24 hours prior to being detected/identified through a culturing methodology. In comparison, spectroscopy will allow its detection within minutes. In summary, identification and detection of micro-organisms is possible through the entire spectral range and/or within specific regions of the spectral range.

After the input spectroscopic data 220 is processed with the previous sequence of pre-processing algorithms it is provided as an input into multiple different machine learning modules that include one or more machine learning algorithms at a training step 255. Aptly, the training dataset of the spectroscopic data 220 (defined in the splitting step 230) is provided as an input into the machine learning modules to provide initial trained machine learning models. Aptly, the validation dataset of the spectroscopic data 220 (defined in the splitting step 230) is then provided as an input into the initial trained machine learning models to provide trained machine learning models. The modules may be used to determine bio-markers from the spectroscopic data that are associated with known microorganisms such that the trained machine learning models are partly based on the determined bio-markers. Aptly, the bio-markers may be spectral peak position, spectral shift, spectral shape, spectral intensity, or spectral area associated with a known microorganism. Optionally, all the modules (which store the machine learning algorithms) used process the data with different mathematical models. Aptly, the pre-processed input training spectroscopic data 220 may be provided as an input into a linear discriminant analysis module, a support vector machine module, a logistic regression module, a K nearest neighbours module, a random forest module, a partial least squares module, an artificial neural network module or a convolutional neural network module. Most of the algorithms learn their parameters through training and others compare the spatial position in the feature space of new samples to previous input samples to predict the new sample class. Once the modules containing the machine learning algorithms are trained, the resulting trained machine learning models can predict a class (e.g. bacteria strain) of new samples processed in a similar way to the training spectroscopic data. That is to say that spectroscopic data associated with a biological sample containing one or more unknown microorganisms may be provided as an input into the trained machine learning models in order to identify what microorganisms are present in that sample.

As discussed above, all the models are trained using the X, y matrix with size n×m+1. To predict a class/microorganism (e.g. bacteria strain) using the trained machine learning models from spectroscopic data of a new biological sample the user and/or a pre-programmed algorithm processes the amplitudes of each wavelength as in the matrix X (without the appended class label y), inputs them to the trained model(s) and obtains the predicted microorganism (e.g. bacteria strain) as an output.

Aptly, the machine learning algorithms used are multinomial classifiers, able to predict more than two classes (e.g. bacteria strains). As a model output each sample is assigned to one and only one label. As mentioned before, to train the models, processed data is given to the models for them to learn their parameters/spatial mapping. Once the algorithms are trained and a trained machine learning model is provided, they predict the class of new data. The way each mathematical model works is explained below.

K Nearest Neighbours

K nearest neighbours is a lazy learning algorithm which does not have the training phase hence does not create a general model. Instead it stores all the training data points in a high-dimensional space. To label a new point, it looks at the k neighbour points closest to that new point and assigns the predicted class using a majority vote of its k nearest neighbours. The class sklearn.neighbors.KNeighborsClassifier can be imported from sklearn package. In the methodology according to certain embodiments of the present invention k was set up to 7 and other parameters were left at default.

The mathematics behind K-nearest Neighbours classifier that the source code uses is as follows. The k-nearest neighbour classifier can be viewed as assigning the k nearest neighbours a weight 1/k and all others 0 weight. This can be generalised to weighted nearest neighbour classifiers. That is, where the i^{th} nearest neighbour is assigned a weight wₙᵢ, with Σᵢ₌₁ⁿ wₙᵢ. An analogous result on the strong consistency of weighted nearest neighbour classifiers also holds.

Let cₙ^{wnn} denote the weighted nearest classifier with weights {wₙᵢ}ᵢ₌₁ⁿ. Subject to regularity conditions on the class distributions the excess risk has the following asymptotic expansion:

[symbol]

(C^{wnn})− [symbol] (C^{Bayes})=(B₁sₙ²+B₂tₙ²){1+o(1)},

For constants B₁ and B₂ where:

$${s}_{n}^{2} = {\sum }_{i = 1}^{n} {w}_{ni}^{2} and {t}_{n} = {n}^{- 2 / d} {\sum }_{i = 1}^{n} {w}_{ni} \{ {i}^{1 + 2 / d} - {( i - 1 )}^{1 + 2 / d} \}$$

The optimal weighting scheme {wₙᵢ*}ᵢ₌₁ⁿ, that balances the two terms in the display above is given as follows:

$${k}^{*} = ⌊ {Bn}^{\frac{4}{d + 4}} ⌋ {w}_{ni}^{*} \frac{1}{{k}^{*}} [ 1 + \frac{d}{2} - \frac{d}{2 {k}^{* 2 / d}} \{ {i}^{1 + 2 / d} - {( i - 1 )}^{1 + 2 / d} \} ] for i = 1 , 2 , … , {k}^{*} {w}_{ni}^{*} = 0 for i = {k}^{*} + 1 , … , n .$$

and

With optimal weights the dominant term in the asymptotic expansion of the excess risk is:

$$𝒪 ( {n}^{- \frac{4}{d + 4}} ) .$$

According to certain embodiments of the present invention, using K nearest neighbours the spectroscopic data may be processed as follows:

```
  >>> dataframe = pd.read_csv(“file.csv”, header=None) - Reads the
  main matrix
  >>> dataset = dataframe.values
  >>> X = dataset[:,0:n].astype(float) - defines the X values where n is
  the columns in the main file
  >>> Y = dataset[:,n] - defines the Y values
  >>> from sklearn.neighbors import KNeighborsClassifier - calls the
  function from the package
  >>> neigh = KNeighborsClassifier(n_neighbors=3) - The function that
performs the maths to find the nearest neighbours.
```

Linear Discriminant Analysis (LDA)

Suppose that each of C classes has a mean μi and the same covariance Σ. Then the scatter between class variability may be defined by the sample covariance of the class means:

$${\sum }_{b} = \frac{1}{C} {\sum }_{i = 1}^{C} ( {μ}_{i} - μ ) {( {μ}_{i} - μ )}^{T}$$

where μ is the mean of the class means. The class separation in a direction

$$\vec{w}$$

in this case will be given by:

$$S = \frac{{\vec{w}}^{T} {\sum }_{b} \vec{w}}{{\vec{w}}^{T} \sum \vec{w}}$$

This means that when

$$\vec{w}$$

is an eigenvector of Σ⁻¹Σ_{b} separation will be equal to the corresponding eigenvalue.

According to certain embodiments of the present invention, using LDA the spectroscopic data may be processed as follows:

>>> dataframe = pd.read_csv(“file.csv”, header=None) - Reads the main
matrix
>>> dataset = dataframe.values
>>> X = dataset[:,0:n].astype(float) - defines the X values where n is the
columns in the main file
>>> Y = dataset[:,n] - defines the Y values
>>> import numpy as np
>>> from sklearn.discriminant_analysis import
LinearDiscriminantAnalysis - calls the LDA function from the package
>>> clf = LinearDiscriminantAnalysis( ) - perfoms the calculations
>>> clf.fit(X, y) - perfoms the calculations given the X and Y values.

Support Vector Machines (SVM)

SVM assigns a linear or a non-linear hyperplane which maximises distance between the classes and ensures their best separation. For the study problem, a few implementations have been used as follows.

Class sklearn.svm.SVC using a linear kernel with the remaining parameters left at default. The class uses a one-vs-the-rest scheme which means that internally there are several binary classifiers that predicts the class against all the other classes.

Class sklearn.multiclass.OneVsRestClassifier( ) is a wrapper for the Class sklearn.svm.LinearSVC used as an estimator. This method is fitting one classifier per class. This class behaves similarly to SVC with the linear kernel, but in this implementation the kernel liblinear was used.

Class sklearn.linear_model.SGDClassifier applies linear classifiers with stochastic gradient descent—the gradient of the loss is estimated at each sample at a time. The model parameters are updated in the direction of decreasing loss. This class by default uses Support Vector Machine Classifier. The number of iterations have been set up at 1000; other parameters have been left at default.

The open source packages are based in the following open sources;
https://www.csie.ntu.edu.tw/˜cjlin/papers/linear_oneclass_SVM/siam.pdf
https://www.csie.ntu.edu.tw/˜cjlin/papers/quadworkset.pdf

According to certain embodiments of the present invention, using SVM the spectroscopic data may be processed as follows:

>>> dataframe = pd.read_csv(“file.csv”, header=None) - Reads the main matrix
>>> dataset = dataframe.values
>>> X = dataset[:,0:n].astype(float) - defines the X values where n is the columns in the
main file
>>> Y = dataset[:,n] - defines the Y values
>>> import numpy as np
>>> from sklearn.svm import LinearSVC
>>> from sklearn.multiclass import OneVsRestClassifier- calls the SVM function from
the package
>>> ovsr =OneVsRestClassifier
(LinearSVC(penalty=‘l2’,random_state=0,max_iter=40000,dual=True,tol=0.0001,
C=1.0)) - perfoms the calculations tha finds the closest distance between
samples to classify them.

Logistic Regression

Multionomial logistic regression is an extension of binary logistic regression which uses cross-entropy loss to predict the probability that an input sample belongs to a class. The class sklearn.linear_model.LogisticRegression has been imported from sklearn packages with multi_class parameter set at multinomial and solver set to lbfgs, leaving remaining parameters at default including L2 regularization.

In a simple form, logistic regression is based on a sigmoid function that brings any real value between 0 and 1 and it is defined as:

$$σ ( t ) = \frac{1}{1 + {e}^{- t}}$$

On the other hand, t within the function is a linear function:

t=β₀+β₁x

Hence, the logistic equation will become:

$$f ( x ) = \frac{1}{1 + {e}^{- ( {β}_{\text{?}} + {β}_{\text{?}} x )}}$$

$$\text{?} \text{indicates text missing or illegible when filed}$$

This formula, overall, provides the separation of the data between 0 and 1.

According to certain embodiments of the present invention, using logistic regression the spectroscopic data may be processed as follows:

>>> dataframe = pd.read_csv(“file.csv”, header=None) - Reads the main
matrix
>>> dataset = dataframe.values
>>> X = dataset[:,0:n].astype(float) - defines the X values where n is the
columns in the main file
>>> Y = dataset[:,n] - defines the Y values
>>> from sklearn.linear_model import LogisticRegression - calls the
logistic regression function from the package
>>> clf = LogisticRegression(random_state=0, solver=‘lbfgs’,
multi_class=‘multinomial’) - performs - performs the calculations finding
the classes separating the data between 0 and 1 to execute the
classification.

Random Forest

Random forest is an ensemble learning method based on collection of decision trees as estimators—each trained on the subsample of the data. The model assigns to the sample the most frequent label across all estimators' predictions. Underlying Decision Tree algorithm structure resembles a tree. The algorithm starts at a root and splits at feature nodes (starting with the features that divides the training data more uniformly) into branches depending on the feature value. The algorithm ends in the leaf which represents a classification label. Rules are exhaustive and mutually exclusive so each new sample is classified according to learnt ruleset. The class sklearn.ensemble.RandomForestClassifier can be imported from sklearn package to build Random Forest classifier with 10 decision trees and entropy criterion. Remaining parameters have been left at default values.

The algorithm that the python software uses is based on the algorithm created by Breiman, “Random Forests”, Machine Learning, 45(1), 5-32, 2001. Found in https://link.springer.com/article/10.1023/A:1010933404324

The whole package of Random forest is based on the mathematical calculation of this article.

According to certain embodiments of the present invention, using random forest the spectroscopic data may be processed as follows:

>>> dataframe = pd.read_csv(“file.csv”, header=None) - Reads the main
matrix
>>> dataset = dataframe.values
>>> X = dataset[:,0:n].astype(float) - defines the X values where n is the
columns in the main file
>>> Y = dataset[:,n] - defines the Y values
>>> from sklearn.ensemble import RandomForestClassifier- calls the
Random forest function from the package
>>> clf = RandomForestClassifier(max_depth=2, random_state=0) -
performs the calculations finding the classes separating the branches
finding the best separation among them.

Artificial Neural Networks

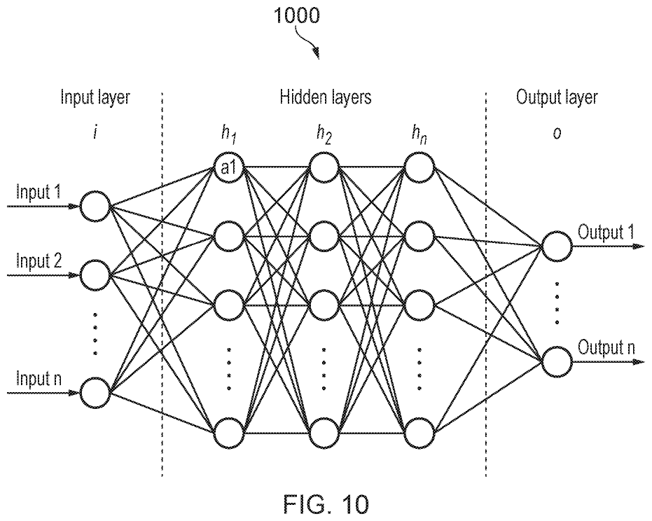

An artificial neural network (ANN) is a computing system that simulates the way the human brain analyzes and processes information. A dense layer may be used to predict microbial samples. An artificial neural network 1000 may be represented as shown in FIG. 10.

The input layer of the neural network are all the X variables available from the input matrix. Each unit and subunit are interconnected and these represent dendrites, the Neural network varies on hidden layers according to the provided data. Each subunit of the hidden layers are ruled by mathematical equations called activation function, which is responsible for processing the sum of the weighted input of each subunit in the neural network architecture to provide a predicted output. The model uses Dense function from keras library. Dense implements the operation: output=activation(dot(input, kernel)+bias) where activation is the element-wise activation function passed as the activation argument, kernel is a weights matrix created by the layer, and bias is a bias vector created by the layer (only applicable if use_bias is True). The model uses a rectified linear activation function (ReLU) and a uniform kernel, responsible for the training process.

The open source Neural networks packages can be found on:
https://keras.io/api/layers/core_layers/dense/

According to certain embodiments of the present invention, using ANNs the spectroscopic data may be processed as follows:

```
  >>> dataframe = pd.read_csv(“file.csv”, header=None) - Reads
  the main matrix
  >>> dataset = dataframe.values
  >>> X = dataset[:,0:n].astype(float) - defines the X values where n is
  the columns in the main file
  >>> Y = dataset[:,n] - defines the Y values
  >>> from keras.models import Sequential - calls sources from the
  package needed for ANN
  >>> from keras.layers import Dense - calls sources from the package
  needed for ANN
  >>> from keras.wrappers.scikit_learn import KerasClassifier - calls
sources from the package needed for ANN
  >>> def baseline_model( ): - From this line onwards, the function to
  create an ANN is defined, here the number of layers are defined.
```

| | |
|---|---|
| >>> | # create model |
| >>> | model = Sequential( ) |
| >>> | model.add(Dense(1020, input_dim=510, activation=“relu |
| | kernel_initializer=“uniform”)) |
| >>> | model.add(Dense(2, activation=“softmax”, |
| | kernel_initializer=“uniform”)) |
| >>> | model.compile(loss=‘categorical_crossentropy’, |
| | optimizer=‘adam’, metrics=[‘accuracy’]) |

>>> return model

Convolutional Neural Networks

Convolutional neural networks (CNNs) capture features from data. The CNN model creates a convolution kernel that is convolved with the layer input over a single spatial (or temporal) dimension to produce a tensor of outputs.

For spectra, where images are not used a 1D convolution layer may be used. This layer creates a convolution kernel that is convolved with the layer input over a single spatial (or temporal) dimension to produce a tensor of outputs.

The open source Neural networks packages can be found on:
https://keras.io/api/layers/convolution_layers/convolution1d/

According to certain embodiments of the present invention, using CNNs the spectroscopic data may be processed as follows:

>>> dataframe = pd.read_csv(“file.csv”, header=None) - Reads the main matrix
>>> dataset = dataframe.values
>>> X = dataset[:,0:n].astype(float) - defines the X values where n is the columns in the
main file
>>> Y = dataset[:,n] - defines the Y values
>>> from keras import models - calls sources from the package needed for the CNN
>>> from keras import layers - calls sources from the package needed for the CNN
>>> from keras.layers import Conv1D - calls sources from the package needed for the
CNN
>>> from keras.layers import Dense - calls sources from the package needed for the
CNN
>>> from keras.layers import Flatten - calls sources from the package needed for the
CNN
>>> from keras.layers import MaxPooling1D - calls sources from the package needed
for the CNN
>>> from keras.layers import Activation, Dropout - calls sources from the package
needed for the CNN
>>> from keras.layers import Input, Flatten - calls sources from the package needed for
the CNN
>>> from keras.models import Sequential -- calls sources from the package needed for
the CNN
>>> model = Sequential( ) - from this point onwards the model to create the CNN is
defined, the convolutional layers are defined, the functions form the packages calculate
the convoluted layers.
>>> model.add(layers.Conv1D(64, 2, activation=‘relu’, input_shape=(510,1)))
>>> model.add(MaxPooling1D(5))
>>> model.add(Flatten( ))
>>> model.add(Dense(100, activation=‘relu’))
>>> model.add(Dense(2, activation=‘softmax’))#Number of outputs
>>> model.compile
(optimizer=‘rmsprop’,loss=‘categorical_crossentropy’,metrics=[metrics.categorical_accur
acy])

For each model used, certain metrics may be obtained. The models detect the trained samples and the test samples to calculate the predicted samples and metrics. The data may behave differently for each model, hence different models may be used to provide greater flexibility for detecting microorganisms. Obtaining different metrics and comparing each model helps to provide an indication of the most suitable model to predict certain samples.

In certain embodiments, the hyper-parameters, formerly known as features or wavenumbers, used in each algorithm are tailored towards the classification problem using train-validation-test datasets. In addition, in certain embodiments, data pre-processing is included in order to help each algorithm improve their prediction capabilities.

In certain embodiments, the algorithms follow an order depending on whether they are being trained or used for prediction.

While training a model the following order is typically followed: Create feature matrix, append encoded label vector, shuffle samples, split into train/validation/test datasets, standardization (optional; trained only with train dataset), PCA (optional; trained only with train dataset), train machine learning algorithm. The validation and test sets are used to select the best models (pre-processing and ML algorithm) with respect to their prediction capabilities with the current bacteria strains.

While predicting the presence of unknown microorganisms aptly the final model selected is used. The order of the algorithms is typically as follows: Standardisation (if used during training), PCA (if used during training), trained machine learning model(s) (machine learning algorithm prediction). Aptly, each of the machine learning algorithms is a multinomial classification algorithm.

In certain embodiments, the output level is indicated by a confusion matrix that allows to identify the True positives and the Microbes incorrectly predicted on a given class. See example confusion matrix below.

Test Confusion Matrix

| | 1 | 2 | 3 | 4 | 5 | 6 | 7 |
|---|---|---|---|---|---|---|---|
| 1 | [[29 | 1 | 0 | 0 | 0 | 0 | 0] |
| 2 | [0 | 61 | 0 | 0 | 0 | 0 | 0] |
| 3 | [1 | 0 | 30 | 0 | 0 | 0 | 0] |
| 4 | [0 | 0 | 0 | 51 | 0 | 0 | 0] |
| 5 | [0 | 0 | 0 | 0 | 54 | 0 | 0] |
| 6 | [0 | 1 | 0 | 0 | 0 | 45 | 0] |
| 7 | [1 | 5 | 1 | 0 | 0 | 10 | 25]] |

The number on the top and on the left that goes from 1 to 7 are representative of 7 different microorganisms. Inside the confusion matrix are the predicted spectra. The confusion matrix is obtained using the function sklearn.metrics.confusion_matrix by calling the test set and the predicted set. The confusion matrix helps to identify the samples correctly or incorrectly predicted by the machine learning model as will be appreciated by the person skilled in the art. Together with specificity and sensitivity and F1 scores, the efficiency of prediction can be provided. These metrics are obtained by using the function sklearn.metrics.classification_report by calling the test set and the predicted set.

Following the training step 255, a number of trained machine learning models are thereby provided. These trained models may then be provided with input spectroscopic data. The test dataset is used in conjunction with a predicted dataset generated by the model, the function sklearn.metrics.classification_report uses both test dataset and predicted values to calculate the metrics in order to generate a score indicative of an accuracy of the specific machine learning models and facilitate a comparison of the scores generated via each model. The provision of this further spectroscopic data into the trained machine learning models is illustrated at model running step 260. In more detail, in the model running step 260, further spectroscopic data is provided into each of a linear discriminant analysis based model 262, a support vector machine based model 264, a linear regression based model 266, a k nearest neighbours based model 268 and a random forest based model 270 to evaluate each of the trained models. It will be appreciated by the person skilled in the art that according to certain embodiments of the present invention more or less trained machine learning models than those shown in FIG. 2 may be provided and evaluated. That is, any numbers of trained machine learning models may be provided and evaluated according to certain embodiments of the present invention. The way the models mathematically function has been described hereinabove.

Using an output from each of the models, a respective score is calculated for each of the trained models. The scores are calculated using the sklearn.metrics.classification_report function. These scores are then compared at a comparison step 280. After comparing the scores, one or more trained models are then selected at a selection step 290. The selected models are those having the highest score indicative of the prediction accuracy for the microorganism associated with the further data input into the trained machine learning models.

In certain embodiments, the method further comprises:
determining at least one score indicative of an identification accuracy of a selected microorganism for each of the plurality of trained machine learning models, wherein optionally the score is a specificity or sensitivity or F1 score or negative predictive value (NPV) or positive predictive value (PPV) or negative predictive agreement (NPA) or positive predictive agreement (PPA).

In certain embodiments, the method further comprises:
responsive to comparing a plurality of scores, each score being associated with a respective one of the plurality of trained machine learning models, selecting a trained machine learning model having a score that is a highest score for the plurality of trained machine learning models, for the selected microorganism.

According to certain embodiments, a library of known micro-organisms (i.e standard organisms identified using currently available techniques) is obtained, and reproducible spectra is obtained to produce a database of spectra, thus each spectra is linked to a microorganism. When predicting unknown microorganisms in a clinical sample the spectra from the clinical sample may be compared with the database (known samples) via the trained machine learning models. The different models may be run simultaneously, since they require around 30 sec on average to acquire a result. Alternatively, the models may be run sequentially or may be run at different times. At the end when the models have finished analysing the data the prediction accuracies between each model will be compared, to determine which model is giving the best prediction. Aptly, the best metric of each model to the same samples to be predicted are compared. The best algorithm may vary with the type of sample and it may be necessary for the final user to provide information as to the nature of the sample the best algorithm to be provided for that sample type. Results of basic laboratory findings such as gram stain result may be used in addition to the system to improve identification. The type of sample may also be important in interpretation for example.

The methodology for predicting the microorganism(s) present in unknown samples will be automated and performed in real time. Table 5 below illustrates this process, where different F1 scores were obtained from the different spectral regions of the analysed microorganisms. The best model to predict certain samples may be chosen from the best metric obtained, where 95% to 100% is indicative of a best score. It is also possible to observe the different models used and the F1 score obtained in the different spectral regions.

Data may also be collected from in use systems to continue to perform analyses on the most effective machine learning algorithm and to continue to refine the system.

TABLE 5
F1 Score

| | Whole Spectral | Fingerprint | High Wavelength |
|---|---|---|---|
| | Region | Region | Region |
| LDA | 88.07% | 91.35% | 86.61% |
| Linear Nu-SVM | 100% | 100% | 99.52% |
| (The Unscrambler) | | | |
| Linear C-SVM | 100% | 100% | 99.47% |
| The Unscrambler) | | | |
| Linear SVM | 99.4% | 99% | 89.1% |
| (Python) | | | |
| One Vs. All SVM | 100% | 100% | 99% |
| Stochastic Gradient | 100% | 100% | 98.1% |
| Descent SVM | | | |
| KNN | 85.6% | 92.7% | 75.6% |
| KNN-PCA | 97% | 98% | 92% |
| LR | 100% | 100% | 98.7% |
| RF | 95.9% | 95.6% | 85.4% |

### Example 1

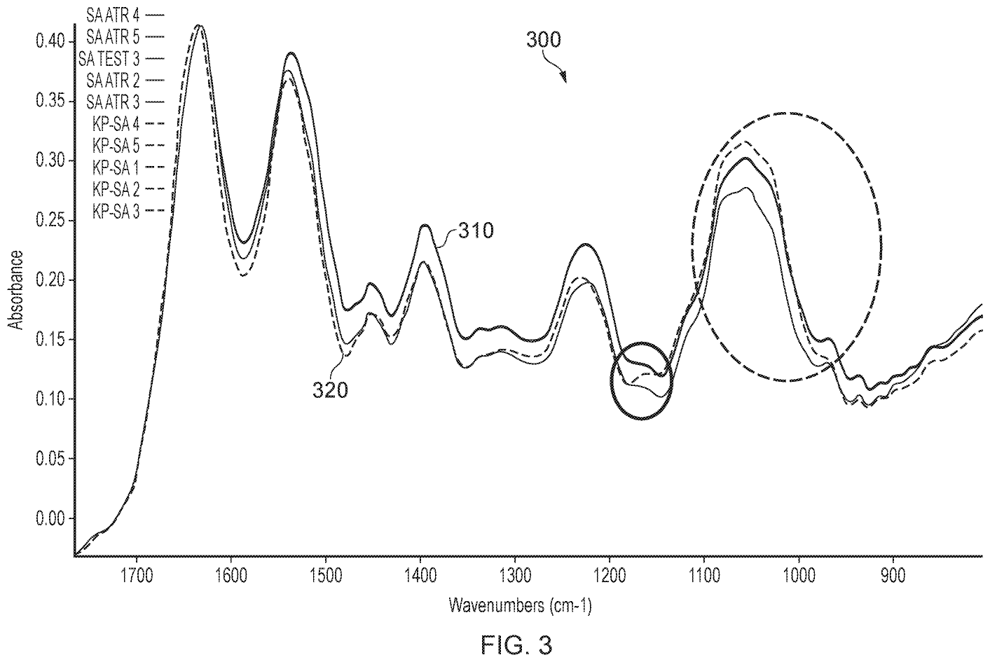

To detect different microorganisms in a sample subtle changes are detected in the spectrogram peaks, as well as shifts that occur in the X (wavenumbers), and Y (Absorbance or Raman Shift) axes. For example, in FIG. 3 a spectrogram 300 is shown in which the spectra 310 of the SA (Staphylococcus aureus) organism and the spectra 320 of a combination of two organisms, KP (Klebsiella pneumoniae) and SA, are compared, subtle changes in the peak shape, form, area, intensity between the spectra regions where presence of a shoulder appears in an ascending form in KP-SA in a constant way, while in SA this shoulder appears in a descending form indicating a different type of molecular interaction (FIG. 3. Smaller blue circle). In addition, different changes can be found in the Y-axis, increment in the intensity/absorbance of the peak (FIG. 3. Larger red circle), and also indicating a different molecular vibration. These characteristics can vary from spectrum to spectrum and manually analysing large number of spectra could be laborious and time consuming. Therefore, these differences are captured in seconds by the trained machine learning models. The models do not indicate where the changes occur but detect and differentiate what species of microorganism a certain set of spectra corresponds to. Spectral peak position, shift, shape, intensity and area analysis can reveal a number of important classifiers able to provide qualitative and quantitative data.

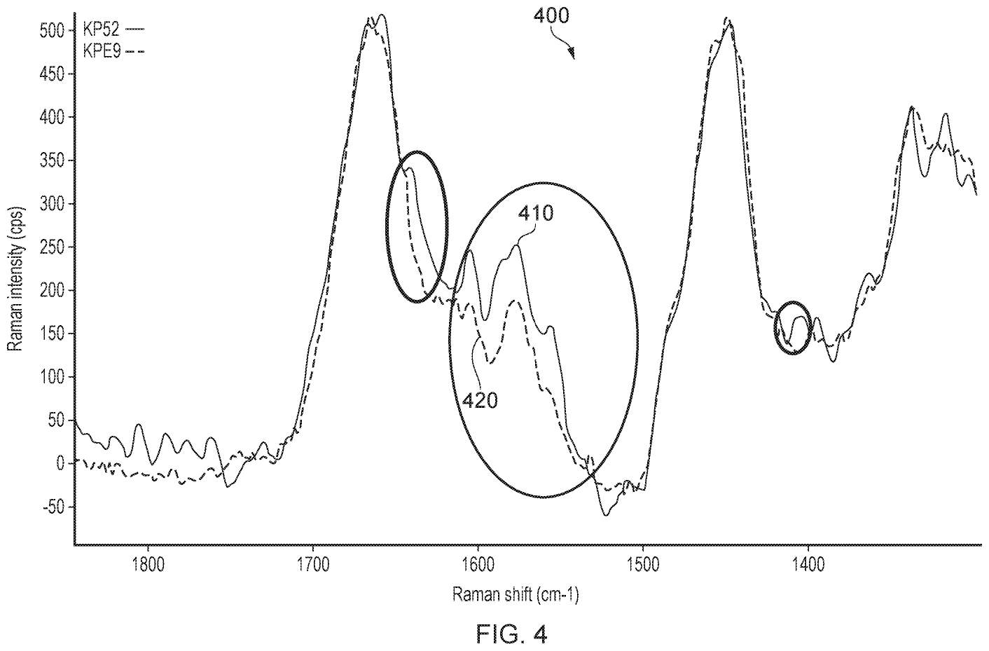

On the other hand, resistance patterns can be identified as shown in FIG. 4. The smaller dark blue circles indicate unique peaks present in the KP52 (Klebsiella pneumoniae serotype 52) spectrum 410, but absent in the KPE9 (Klebsiella pneumoniae serotype E9) spectrum 420. This identifies the microorganism as a carbapenemase resistant strain. The presence of unique peaks among different microorganisms can be more than one. Once the unique peaks present in certain microorganisms have been identified, the region in which these changes are expected to be seen is restricted and processed in the previously created trained machine learning models. The trained machine learning models thereby allows prediction of which microorganism is present in the biological sample.

### Example 2—SARS-COV-2

During SARS-Cov-2 IR spectroscopy analysis two different samples may be collected:
Nasal swabs—Nasal swabs submerged into viral transport media.
Saliva Samples—Saliva from patients collected into a falcon tube. Saliva is flagged as an important biofluid to quickly analyze and assess respiratory diseases caused by viruses.

Nasal Swabs Results

Nasal swab IR spectroscopy analysis was compared with “Golden standard” PCR methods (Panther PCR assay, and Cepheid PCR assay), where it was possible to identify a Positive or Negative sample. A simple binary classification was performed using two different types of analysis; supervised and unsupervised analysis.

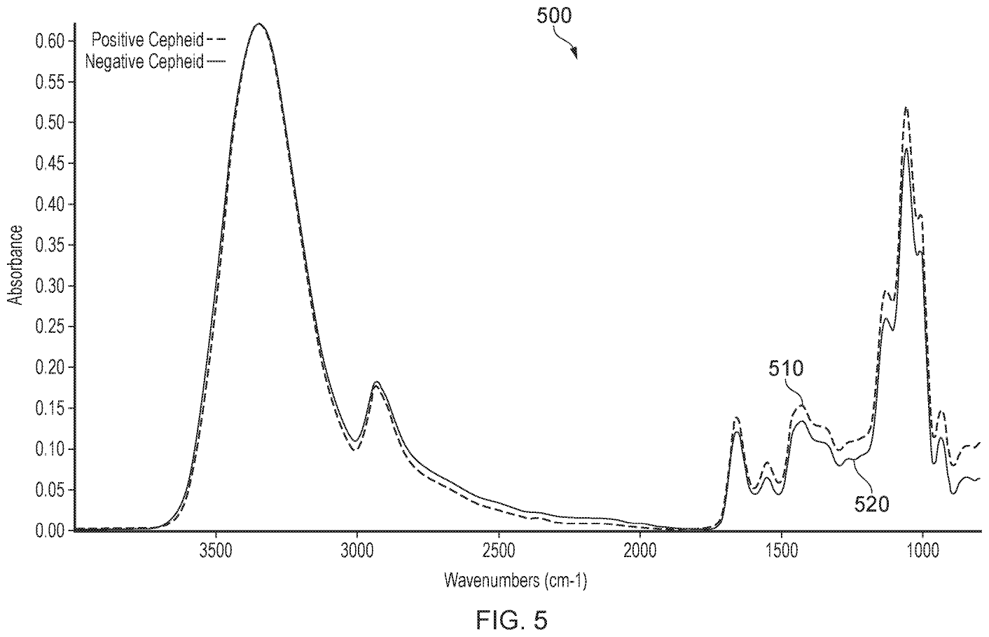

FIG. 5 illustrates spectra 500 of COVID positive 510 and COVID negative 520 detected nasal swabs assessed with Panther PCR assay to detect coronavirus.

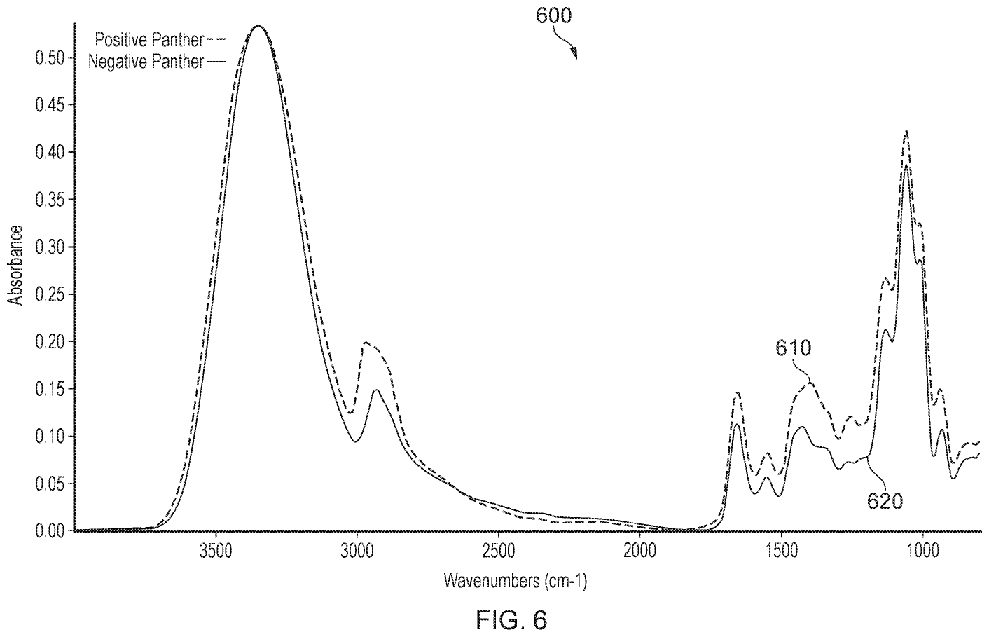

FIG. 6 illustrates spectra 600 of COVID positive 610 and COVID negative 620 detected nasal swabs assessed with Cepheid PCR assay to detect coronavirus.

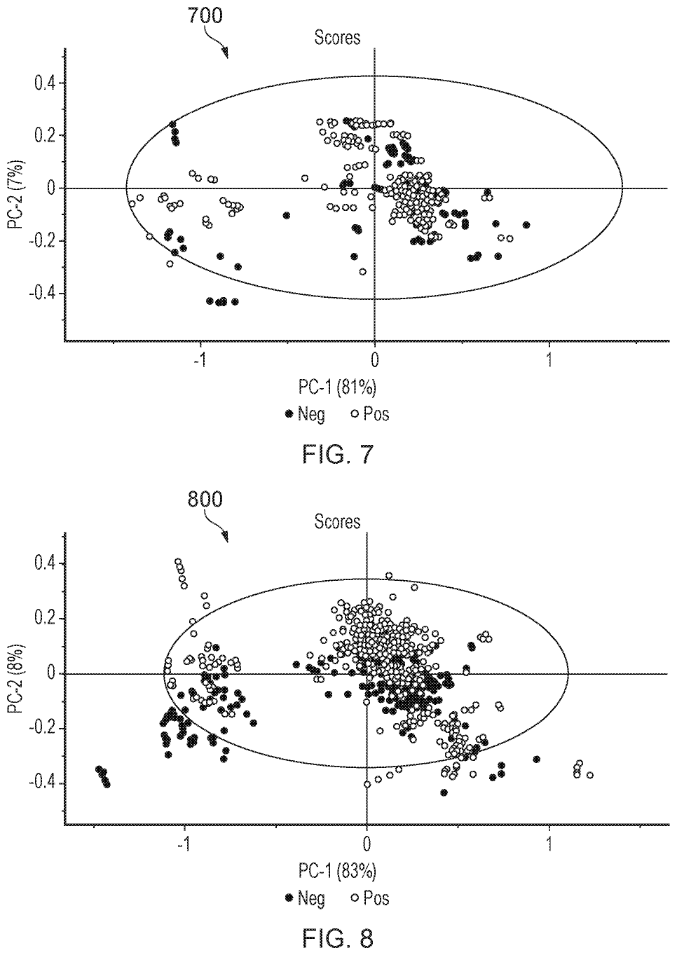

As seen in the PC-1 vs. PC-2 graph 700 of FIG. 7 and the PC-1 vs. PC-2 graph 800 of FIG. 8 (Unsupervised analysis), there is a clear separation between COVID positive (in red) and COVID negative (in blue), however, it is visible that some negative classified samples, as well as positive samples that are overlayed on both clusters. Unsupervised methods help to visualize data. Visualizing the data helped to assess the feasibility of performing a supervised method to classify the data. A supervised method gives control to the user over the labels and methods to classify.

A trained support vector machine (SVM) based model was provided with spectroscopic data from the spectra of both Panther and Cepheid samples (see FIG. 5 and FIG. 6) to classify the data. After running the SVM model the following metrics were obtained.
Panther samples:
   Specificity: 100% of prediction
   Sensitivity: 86% of prediction
Cepheid samples:
   Specificity: 95% of prediction
   Sensitivity: 95% of prediction

For the clinical setup the Positive Percent Agreement (PPA), Negative Percent Agreement (NPA), Positive Predictive Value (PPV), and Negative Predictive Value (NPV), should ideally be obtained in order to validate the feasibility of the study for the clinical setup. These results are shown below. The PPA and NPA, are the performance characteristics in comparison with a previous study, in this case PCR assays. PPA is the proportion of comparative/reference method positive results in which the test method result is positive. NPA is the proportion of comparative/reference method negative results in which the test method result is negative.

When evaluating the feasibility or the success of a screening program, one should also consider the positive and negative predictive values. PPV is the probability that subjects with a positive screening test truly have the disease. NPV is the probability that subjects with a negative screening test truly don't have the disease. These metrics consider the current COVID-19 prevalence set to 1%.

PPV and NPV are obtained as follows:

PPV=(sensitivity*prevalence)/sensitivity*prevalence+(1−specificity)*(1−prevalence)

NPV=(specificity*(1−prevalence))/(1−sensitivity)*prevalence+specificity*(1−prevalence)
Panther samples:
   NPA: 88.88%
   PPA: 100%
   NPV: 99.12% to 99.98%
   PPV: 100%
Cepheid samples:
   NPA: 95.23%
   PPA: 95.23%
   NPV: 99.66% to 99.99%
   PPV: 2.89% to 57.83%

In the Panther samples it is possible to identify whether a patient is diagnosed or not with SARS-Cov-2. Whereas in Cepheid samples, it was more probable to find that a negative diagnosis in a patient sample. The test is more likely to tell that a patient does not have SARS-COV-2.

Saliva Samples

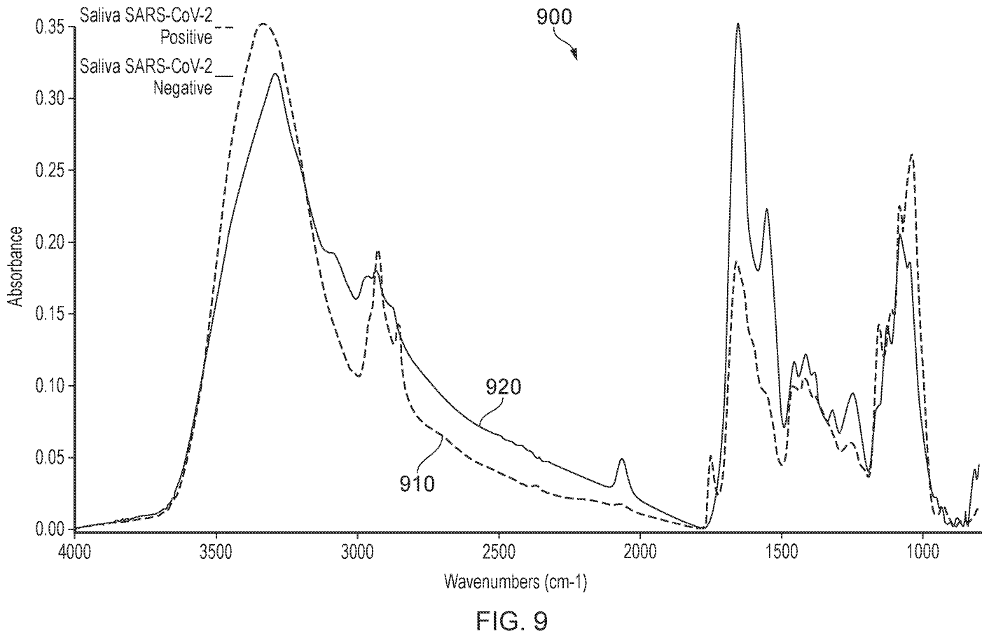

The spectra 900 illustrated in FIG. 9 are from COVID positive 910 and COVID negative 920 saliva samples of patients. The collected samples present substantial differences along the spectra. The spectra of saliva samples show more differences among the spectra in comparison with the nasal swabs submerged in VTM and so these spectra may be used in certain embodiments of the present invention.

Using the methods according to certain embodiments of the present invention it may be possible to detect and identify bacteria directly from samples in under 1 hour. In addition we are able to directly tell the difference between the same strain of bacteria with different resistance phenotypes, whereas using current routine methods this can take up to 48 hours. In case of pathogens such as viruses the identification of viruses present in tissues will be reduced having a 30 minutes time frame of detection.

When obtaining the different collections of spectra, the algorithms used evaluate and analyse the changes that occur between different wavelengths in the infrared (4000-400 cm⁻¹), and Raman (4000-400 cm⁻¹) spectral region. In other words, what the algorithms study are the changes on specific molecular vibration signals that identify and differentiate one microorganism from another (even being mixed in the same sample), these specific signals are called bio-markers. Bio markers are found in both the high wavelength range and the fingerprint region, in both IR and Raman spectroscopic techniques. A database contains the spectra of different available microorganisms and after the spectra collection process is finished it will be used to run the machine learning algorithms in order to compare and predict against all kinds of unknown samples. The whole process may be completed in less than 30 minutes and can be evaluated by different metrics such as specificity, sensitivity, and F1 score.

Certain embodiments of the present invention enables the identification of microorganisms down to the species level (not just to the genus level). As an example, the genus Staphylococcus spp contains Staphylococcus aureus, a cause of serious skin and wound infection and Staphylococcus epidermdidis which lives normally on the skin and would not be treated. The identification to species level requires a high level of analysis including spectral interpretation using various spectral regions as discussed herein. Examples of this speciation are given below with reference to the accompanying figures.

Furthermore, if antibiotic resistant variants cannot be detected during identification of micro-organisms, the advantage of rapid diagnosis may be lost. For example, Staphylococcus aureus is mainly treated using Flucloxacillin. However, if this treatment was used for the methicillin resistant strain of Staphylococcus aureus (MRSA), treatment would be unsuccessful. Thus, it is important that antibiotic resistant strains of a micro-organism can be identified using the methods described herein. The identification of antibiotic resistant strains requires a high level of analysis using various spectral regions as discussed herein. Examples of detection of antibiotic resistant strains of microorganisms are given below with reference to the accompanying figures.

Still furthermore, for rapid detection of microorganisms, it is beneficial for the microorganisms to be detectable from direct biofluid samples taken from a patient (i.e., instead of requiring a pure culture). Further examples of detection of microorganisms from direct biofluid samples are given below with reference to the accompanying figures.

Antibiotic Resistant Bacteria and Differentiation within a Species Level

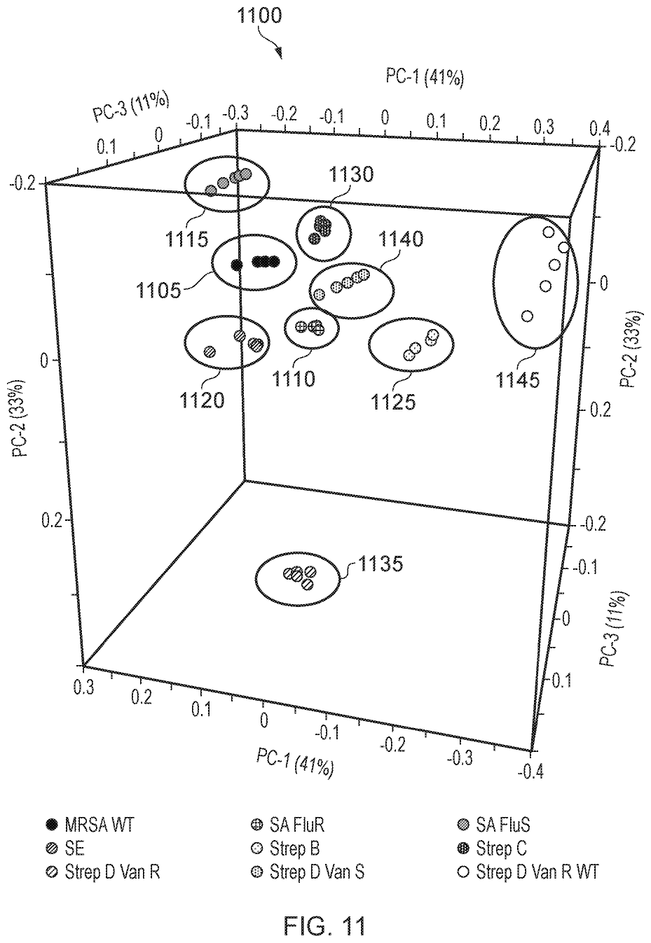

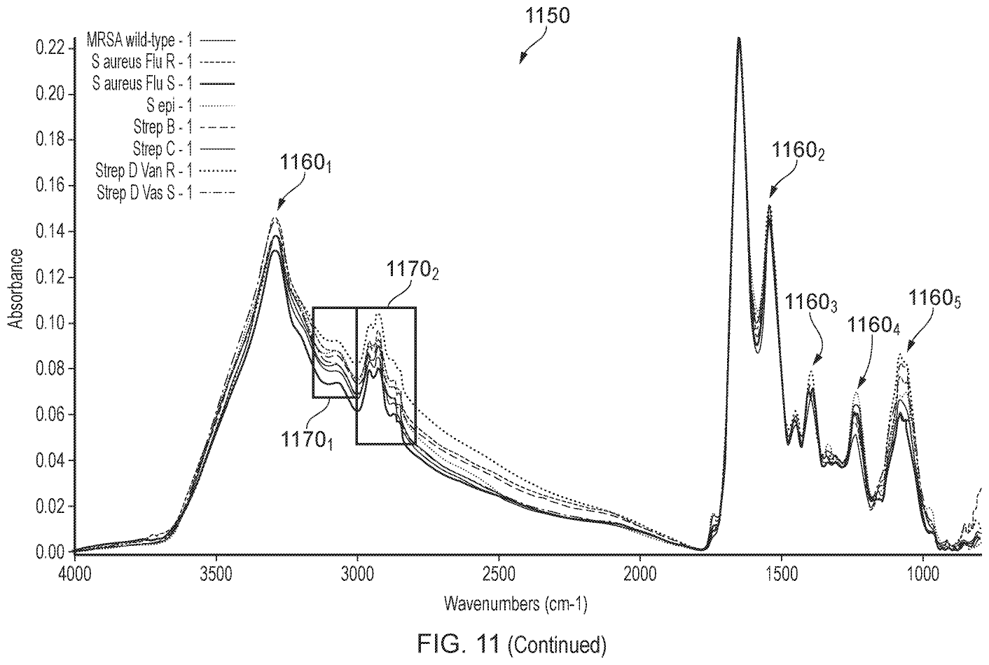

FIGS. 11-15 illustrate data demonstrating both the detection of resistant strains of gram positive bacteria, for example meticillin sensitive v meticllin resistant Staphylococcus aureus as well as Vancomycin sensitive v Vancomycin resistant Enterococcus faecalis (Streptococcus group D) as an exemplar of gram positive bacteria.

These figures also demonstrate speciation within the genus Streptococcus spp, for example Streptococcus Group B v Group C v Group D and within the genus Staphylococcus into Staphylococcus aureus v Staphylococcus epidermidis and Methicillin resistant Staphylococcus aureus Wild Type (MRSA WT). These may be referred to herein as follows:
Staphylococcus aureus Flucloxacillin Resistant and susceptible (SA FluR, SA FluS).
Staphylococcus epidermidis (SE).
Streptococcus B (Strep B).
Streptococcus C (Strep C).
Streptococcus D vancomycin resistant (Strep D VanR), vancomycin resistant wild type (Strep D VanR WT), vancomycin susceptible (Strep D VanS).

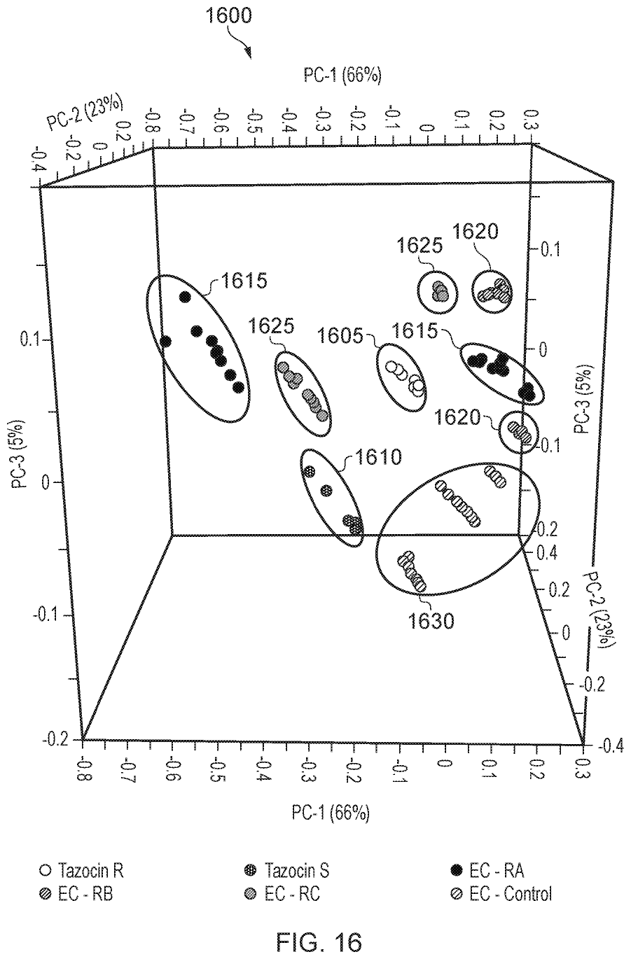

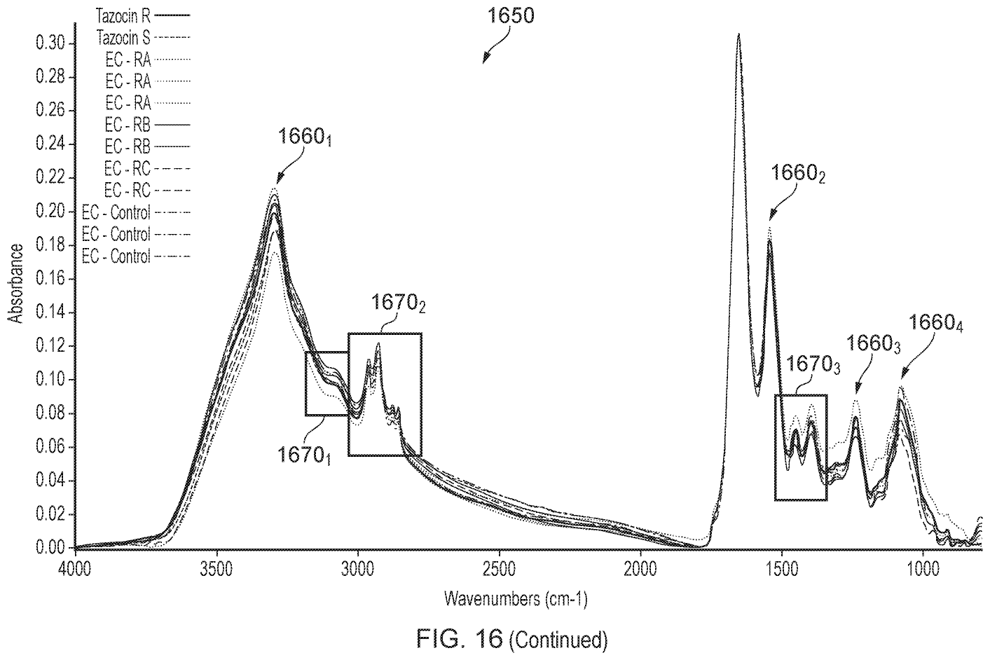

FIGS. 16-20 illustrate data demonstrating detection of resistant strains and speciation of gram negative bacteria. The following examples of gram negative bacteria are shown in these figures:
Escherichia coli tazocin resistant and susceptible (Tazocin R, Tazocin S)
Escherichia coli resistance group A (EC-RA).
   Amikacin, Cefipime, Amoxicillin, Co-amoxiclav, Aztreonam, Ciprofloxacin, Co-trimoxazole, Cefpodoxime, Cefotaxime, Ceftazidime, Gentamicin, Cefuroxime, Tazocin, Tobramycin.
Escherichia coli resistance group B (EC-RB).
   Cefipime, Amoxicillin, Co-amoxiclav, Aztreonam, Ciprofloxacin, Co-trimoxazole, Cefpodoxime, Cefotaxime, Ceftazidime, Gentamicin, Cefuroxime, Tobramycin.
Escherichia coli resistance group C (EC-RC).
   Amoxicillin, Co-amoxiclav, Cefuroxime.
Escherichia coli control group (EC-Control).
   Susceptible to all antibiotics.

FIG. 11 illustrates a discrimination analysis 1100 and absorbance spectra 1150 for certain gram positive bacteria in a spectral region from 4000-400 cm⁻¹. The discrimination analysis 1100 shows data points associated with MRSA WT 1105, SA FluR 1110, SA FluS 1115, SE 1120, Strep B 1125, Strep C 1130, Strep D Van R 1135, Strep D Van S 1140 and Strep D Van R WT 1145 as a function of the calculated PC-1, PC-2 and PC-3 (principal components) as described hereinabove.

To arrive at the discriminant analysis 1100, the data obtained by the vibrational spectroscopy technique is pre-processed, so that unsupervised learning techniques can be used to visualise the data. Discriminant analysis 1100 enables observation of a clear segregation of the data points associated with different microorganisms, thus showing that the clusters of data points are different from each other and allowing data prediction. The plots indicate that a difference in the variance of the data can be obtained from 40% for PC1, 33% for PC2, and 11% for PC3, which allows differentiation of classes. The absorbance spectra 1150 associated with these gram positive bacteria was obtained via FTIR. As can be seen in the spectra 1150, there are a number of peaks 1160₁-1160₅ and a number of regions 1170₁, 1170₂ where differences in the spectra taken from different bacteria are prominent. Differences in the peak shifts 1160₁ and regions 1170₁ and 1170₂ illustrate the differences in the lipid region, peaks 1160₂-1160₄ illustrate amide regions, peak 1160₅ illustrates the differences that occur in the nucleic acid spectral region. By providing spectroscopic data associated with the absorbance spectra 1150 from each species (e.g., SE), some of which may be antibiotic resistant, within this spectral region into one or more machine learning modules, one or more trained machine learning models can be provided as described hereinabove. These trained models are capable of identifying the given microorganism species (e.g., SE) for which they have been given training data. Thus, the models are able to identify if the specific microorganism species (e.g., SE) is present in an unknown sample when provided with spectra from that sample in this spectral region. The spectra from all the classes associated with absorbance spectra 1150 present different shifts in both absorbance and wavenumber and these shifts allow an appropriate differentiation from all classes.

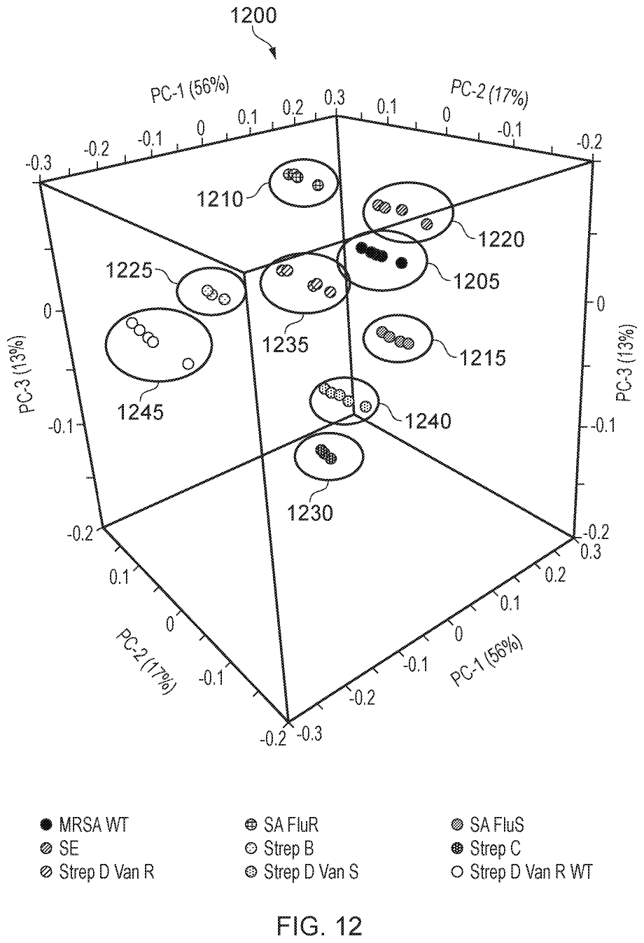

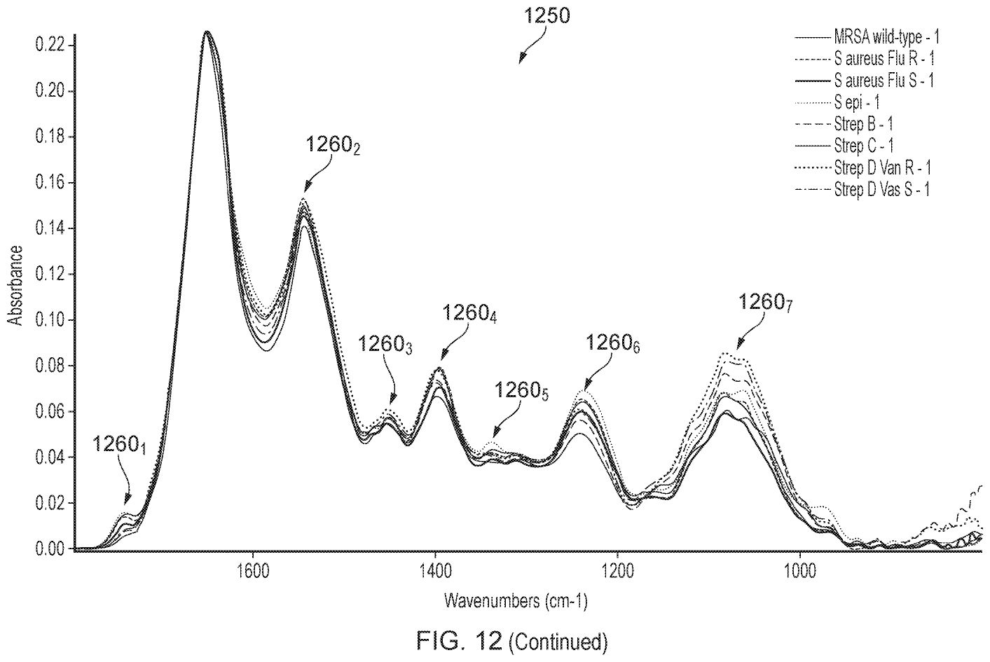

FIG. 12 illustrates a discrimination analysis 1200 and absorbance spectra 1250 for certain gram positive bacteria in a spectral region from 1800-400 cm⁻¹. The discrimination analysis 1200 shows data points associated with MRSA WT 1205, SA FluR 1210, SA FluS 1215, SE 1220, Strep B 1225, Strep C 1230, Strep D Van R 1235, Strep D Van S 1240 and Strep D Van R WT 1245 as a function of the calculated PC-1, PC-2 and PC-3 (principal components) as described hereinabove. To arrive at discriminant analysis 1200, the data from the spectral region from 1800-400 cm⁻¹ obtained by the vibrational spectroscopy technique is pre-processed, so that unsupervised learning techniques can be used to visualise the data. Discriminant analysis 1200 enables observation of a clear segregation of the data points associated with different microorganisms, thus showing that the clusters of data points are different from each other and allowing data prediction. The plots indicate that a difference in the variance of the data can be obtained from 56% for PC1, 17% for PC2, and 13% for PC3, which allows differentiation of classes. The absorbance spectra 1250 associated with these gram positive bacteria was obtained via FTIR.

As can be seen in the spectra 1250, there are a number of peaks 1260₁-1260₇ where differences in the spectra taken from different bacteria are prominent. Differences in the peak shifts 1260₁-1260₆ illustrate amide regions, peak 1260₇ illustrates the differences that occur in the nucleic acid spectral region. By providing spectroscopic data associated with the absorbance spectra 1250 from each species (e.g., SE), some of which may be antibiotic resistant, within this spectral region into one or more machine learning modules, one or more trained machine learning models can be provided as described hereinabove. These trained models are capable of identifying the given microorganism species (e.g., SE) for which they have been given training data. Thus, the models are able to identify if the specific microorganism species (e.g., SE) is present in an unknown sample when provided with spectra from that sample in this spectral region. The spectra from the Fingerprint region from all the classes associated with absorbance spectra 1250 present different shifts in both absorbance and wavenumber and these shifts allow an appropriate differentiation from all classes.

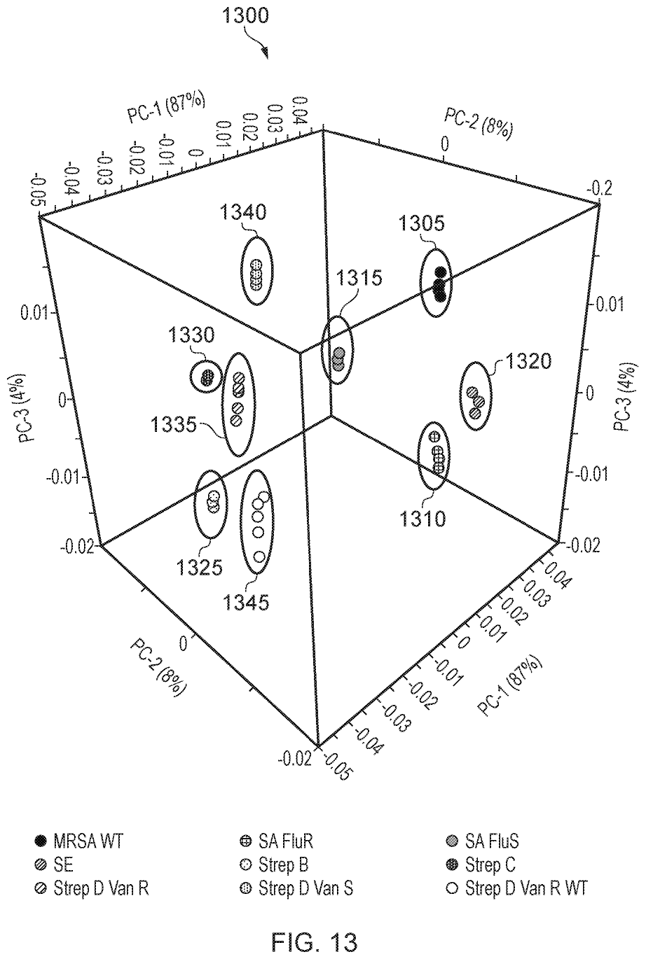

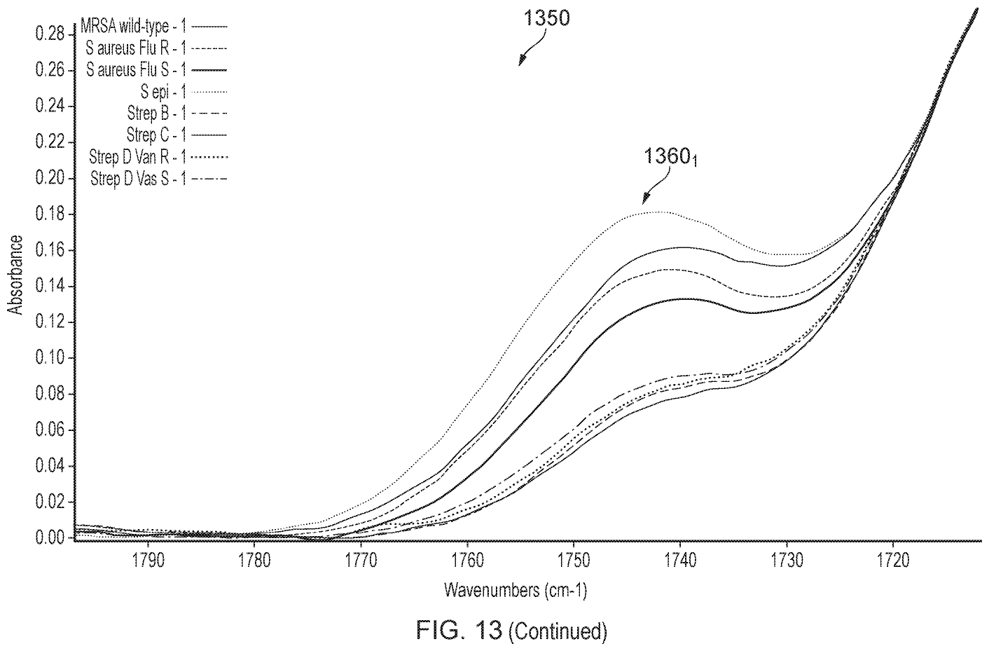

FIG. 13 illustrates a discrimination analysis 1300 and absorbance spectra 1350 for certain gram positive bacteria in a spectral region from 1800-1710 cm⁻¹. The discrimination analysis 1300 shows data points associated with MRSA WT 1305, SA FluR 1310, SA FluS 1315, SE 1320, Strep B 1325, Strep C 1330, Strep D Van R 1335, Strep D Van S 1340 and Strep D Van R WT 1345 as a function of the calculated PC-1, PC-2 and PC-3 (principal components) as described hereinabove. To arrive at discriminant analysis 1300, the data from the spectral region from 1800-1710 cm⁻¹ obtained by the vibrational spectroscopy technique is pre-processed, so that unsupervised learning techniques can be used to visualise the data. Discriminant analysis 1300 enables observation of a clear segregation of the data points associated with different microorganisms, thus showing that the clusters of data points are different from each other and allowing data prediction. The plots indicate that a difference in the variance of the data can be obtained from 87% for PC1, 8% for PC2, and 4% for PC3, which allows differentiation of classes. The absorbance spectra 1350 associated with these gram positive bacteria was obtained via FTIR. As can be seen in the spectra 1350, there is a peak 1360₁ where differences in the spectra taken from different bacteria are prominent. Differences in the peak shift 1360₁ illustrates the differences that occur on the band assigned to C═O stretching of lipids which varies for each of the gram positive bacteria shown in the absorbance spectra 1350. By providing spectroscopic data associated with the absorbance spectra 1350 from each species (e.g., SE), some of which may be antibiotic resistant, within this spectral region into one or more machine learning modules, one or more trained machine learning models can be provided as described hereinabove. These trained models are capable of identifying the given microorganism species (e.g., SE) for which they have been given training data. Thus, the models are able to identify if the specific microorganism species (e.g., SE) is present in an unknown sample when provided with spectra from that sample in this spectral region. The spectra from this Fingerprint sub-region from all the classes associated with absorbance spectra 1350 present different shifts in both absorbance and wavenumber and these shifts allow an appropriate differentiation from all classes.

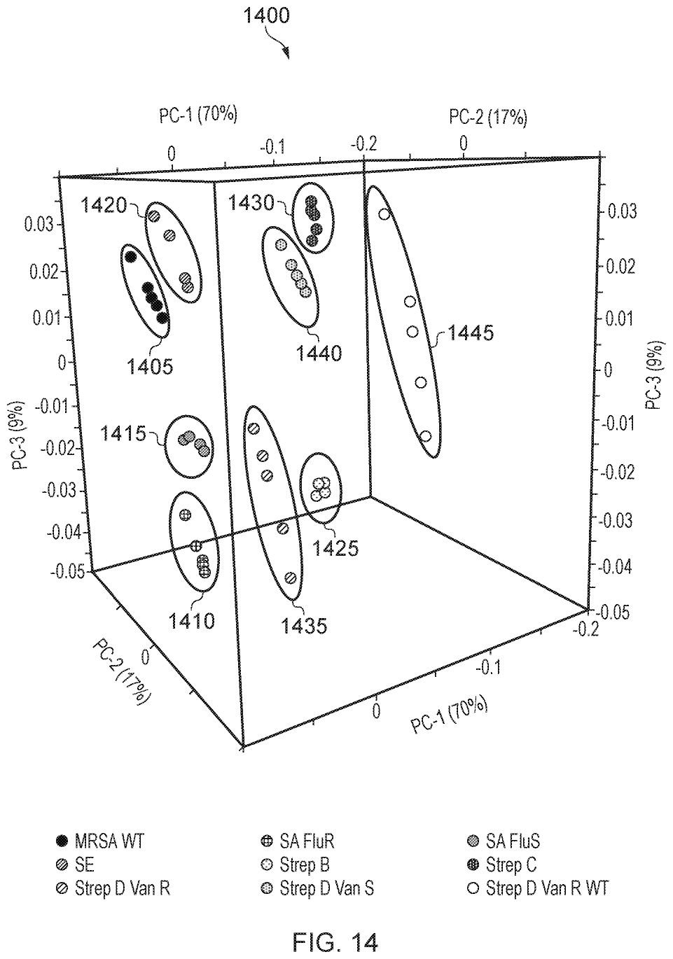

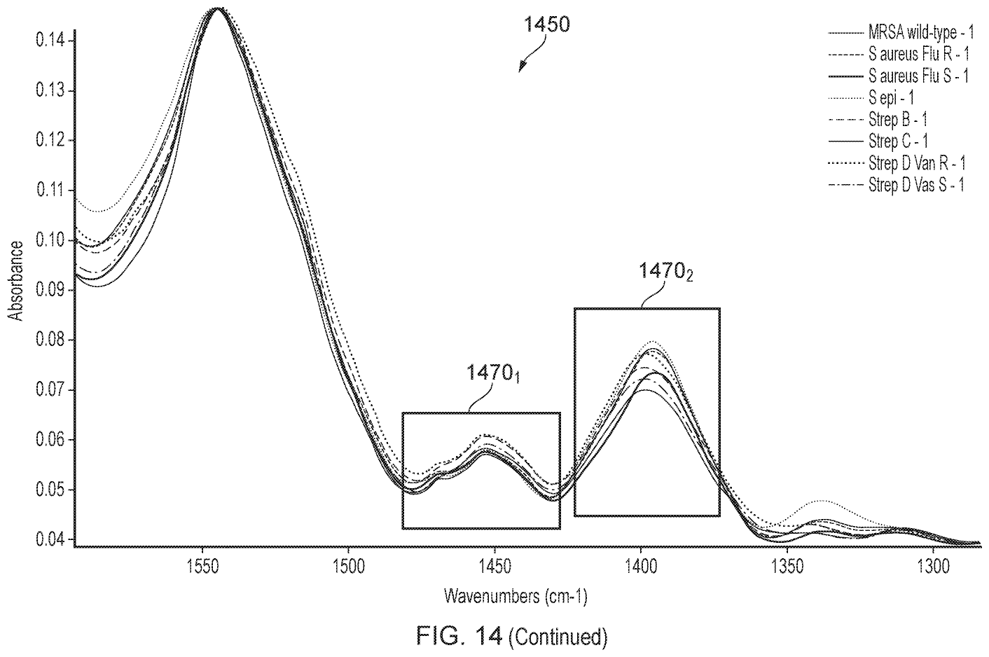

FIG. 14 illustrates a discrimination analysis 1400 and absorbance spectra 1450 for certain gram positive bacteria in a spectral region from 1590-1290 cm⁻¹. The discrimination analysis 1400 shows data points associated with MRSA WT 1405, SA FluR 1410, SA FluS 1215, SE 1420, Strep B 1425, Strep C 1430, Strep D Van R 1435, Strep D Van S 1440 and Strep D Van R WT 1445 as a function of the calculated PC-1, PC-2 and PC-3 (principal components) as described hereinabove. To arrive at discriminant analysis 1400, the data from the spectral region from 1590-1290 cm⁻¹ obtained by the vibrational spectroscopy technique is pre-processed, so that unsupervised learning techniques can be used to visualise the data. Discriminant analysis 1400 enables observation of a clear segregation of the data points associated with different microorganisms, thus showing that the clusters of data points are different from each other and allowing data prediction. The plots indicate that a difference in the variance of the data can be obtained from 70% for PC1, 17% for PC2, and 9% for PC3, which allows differentiation of classes. The absorbance spectra 1450 associated with these gram positive bacteria was obtained via FTIR. As can be seen in the spectra 1450, there are a number of regions 1470₁, 1470₂ where differences in the spectra taken from different bacteria are prominent. Differences in the peak shifts illustrated in absorbance spectra 1450 illustrate different ranges of spectral bands assigned in this fingerprint sub-region, primarily this sub-region includes the amide 2 and 3 spectral bands. A first region 1470₁ shows the differences that occur on the band assigned to CH₃ stretching of lipids and proteins, whereas a second region 1470₂ shows the differences that occur on CH₃ of proteins, an important feature from the amide 3 region which varies for each of the gram positive bacteria shown in absorbance spectra 1450. By providing spectroscopic data associated with the absorbance spectra 1450 from each species (e.g., SE), some of which may be antibiotic resistant, within this spectral region into one or more machine learning modules, one or more trained machine learning models can be provided as described hereinabove. These trained models are capable of identifying the given microorganism species (e.g., SE) for which they have been given training data. Thus, the models are able to identify if the specific microorganism species (e.g., SE) is present in an unknown sample when provided with spectra from that sample in this spectral region. The spectra from this Fingerprint sub-region from all the classes associated with absorbance spectra 1450 present different shifts in both absorbance and wavenumber and these shifts allow an appropriate differentiation from all classes.

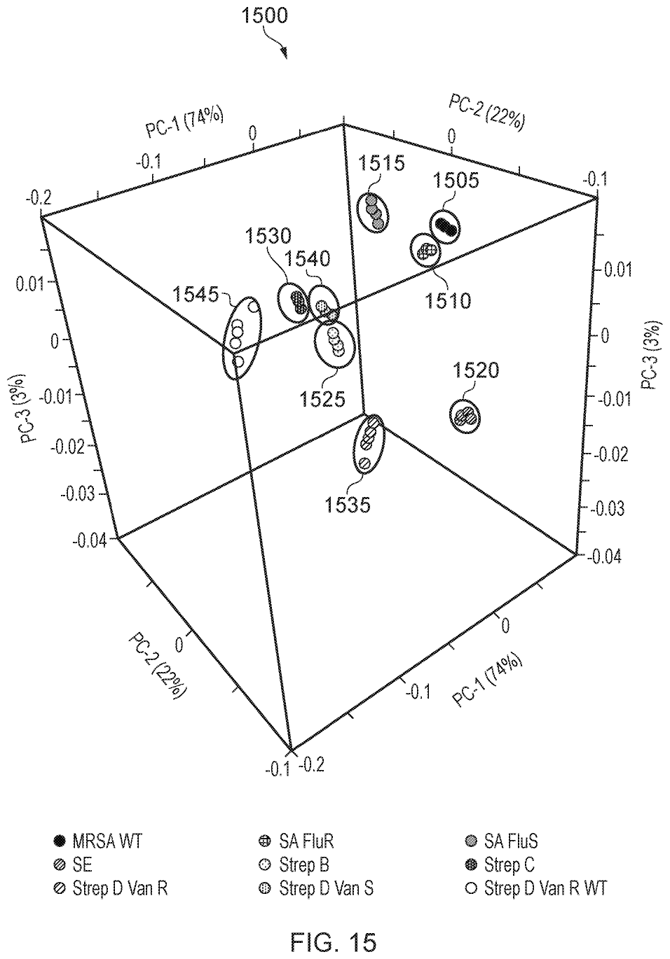

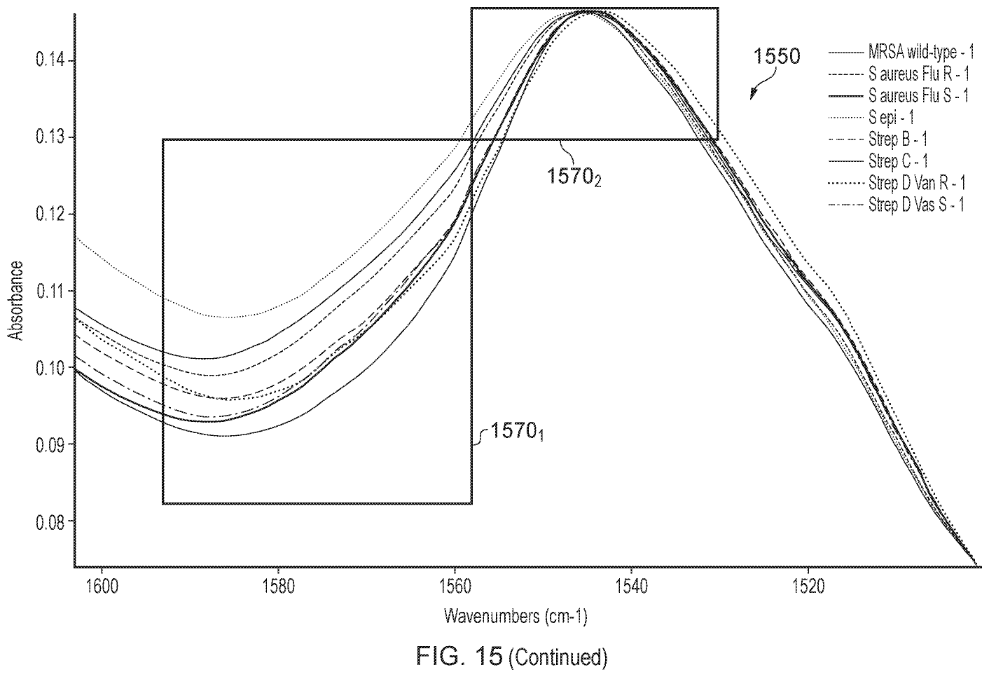

FIG. 15 illustrates a discrimination analysis 1500 and absorbance spectra 1550 for certain gram positive bacteria in a spectral region from 1600-1500 cm⁻¹. The discrimination analysis 1500 shows data points associated with MRSA WT 1505, SA FluR 1510, SA FluS 1515, SE 1520, Strep B 1525, Strep C 1530, Strep D Van R 1535, Strep D Van S 1540 and Strep D Van R WT 1545 as a function of the calculated PC-1, PC-2 and PC-3 (principal components) as described hereinabove. To arrive at discriminant analysis 1500, the data from the spectral region from 1600-1500 cm⁻¹ obtained by the vibrational spectroscopy technique is pre-processed, so that unsupervised learning techniques can be used to visualise the data. Discriminant analysis 1500 enables observation of a clear segregation of the data points associated with different microorganisms, thus showing that the clusters of data points are different from each other and allowing data prediction. The plots indicate that a difference in the variance of the data can be obtained from 74% for PC1, 22% for PC2, and 3% for PC3, which allows differentiation of classes.

The absorbance spectra 1550 associated with these gram positive bacteria was obtained via FTIR. As can be seen in the spectra 1550, there are a number of regions 1570₁, 1570₂ where differences in the spectra taken from different bacteria are prominent. Differences in the peak shifts illustrated in spectra 1550 illustrates the spectral differences on each bacteria class. Regions 1570₁ and 1570₂ describe the shift differences that occur on the band assigned to amide 2 vibration. By providing spectroscopic data associated with the absorbance spectra 1550 from each species (e.g., SE), some of which may be antibiotic resistant, within this spectral region into one or more machine learning modules, one or more trained machine learning models can be provided as described hereinabove. These trained models are capable of identifying the given microorganism species (e.g., SE) for which they have been given training data. Thus, the models are able to identify if the specific microorganism species (e.g., SE) is present in an unknown sample when provided with spectra from that sample in this spectral region. The spectra from this Fingerprint sub-region from all the classes associated with spectra 1550 present different shifts in both absorbance and wavenumber and these shifts allow an appropriate differentiation from all classes.

Performing a discriminative analysis of the data obtained allows the pre-visualisation of the data to be carried out as a preliminary support for the modelling of machine learning models. The visualisation of clusters in different spectral regions not only allows the feasibility and effectiveness in regions other than the full wavenumber region to be observed, but having identified short regions can also help in the creation of vibrational spectroscopic devices with a lower complexity, both structurally and in terms of size.

FIG. 16 illustrates a discrimination analysis 1600 and absorbance spectra 1650 for certain gram negative bacteria in a spectral region from 4000-400 cm⁻¹. The discrimination analysis 1600 shows data points associated with Tazocin R 1605, Tazocin S 1610, EC-RA 1615, EC-RB 1620, EC-RB 1625 and EC-Control 1630 as a function of the calculated PC-1, PC-2 and PC-3 (principal components) as described hereinabove. To arrive at discriminant analysis 1600, the data obtained by the vibrational spectroscopy technique is pre-processed, so that unsupervised learning techniques can be used to visualise the data. Discriminant analysis 1600 enables observation of a clear segregation of the data points associated with different microorganisms, thus showing that the clusters of data points are different from each other and allowing data prediction. The plots indicate that a difference in the variance of the data can be obtained from 66% for PC1, 23% for PC2, and 5% for PC3, which allows differentiation of classes. The absorbance spectra 1650 associated with these gram negative bacteria was obtained via FTIR.

As can be seen in the spectra 1650, there are a number of peaks 1660₁-1660₄ and a number of regions 1670₁-1670₃ where differences in the spectra taken from different bacteria are prominent. Differences in the peak shifts 1660₁ and regions 1670₁ and 1670₂ illustrate the differences in the lipid region, peaks 1660₂ and 1160₃ and region 1670₃ illustrate amide regions, peak 1660₄ illustrates the differences that occur in the nucleic acid spectral region. By providing spectroscopic data associated with the absorbance spectra 1650 from each antibiotic resistant species (e.g., EC-RA) within this spectral region into one or more machine learning modules, one or more trained machine learning models can be provided as described hereinabove. These trained models are capable of identifying the given antibiotic resistant species for which they have been given training data. Thus, the models are able to identify if the specific antibiotic resistant species is present in an unknown sample when provided with spectra from that sample in this spectral region. The spectra from this region from all the classes associated with spectra 1650 present different shifts in both absorbance and wavenumber and these shifts allow an appropriate differentiation from all classes.

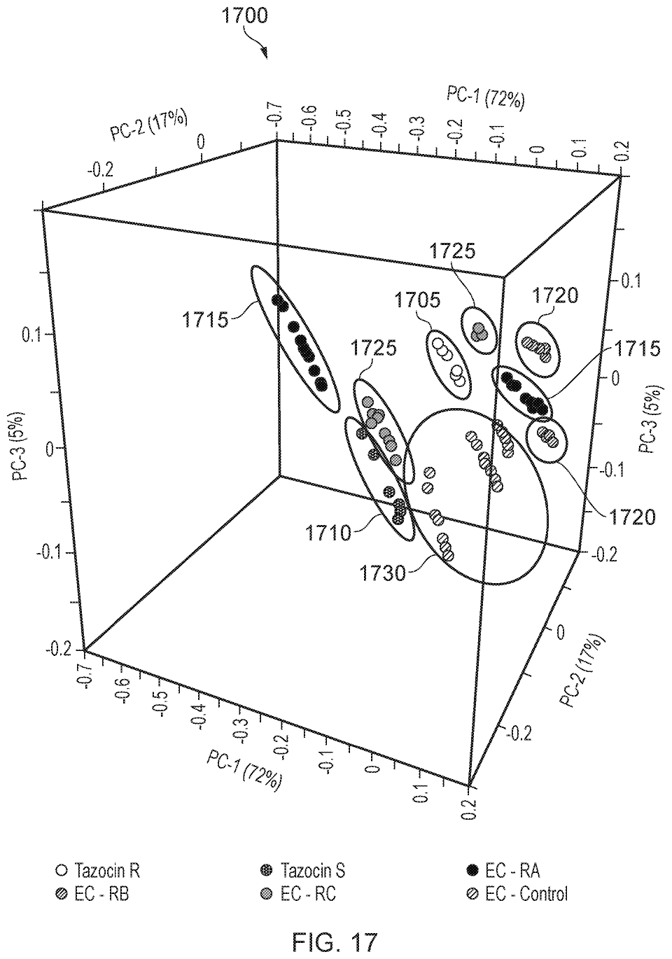

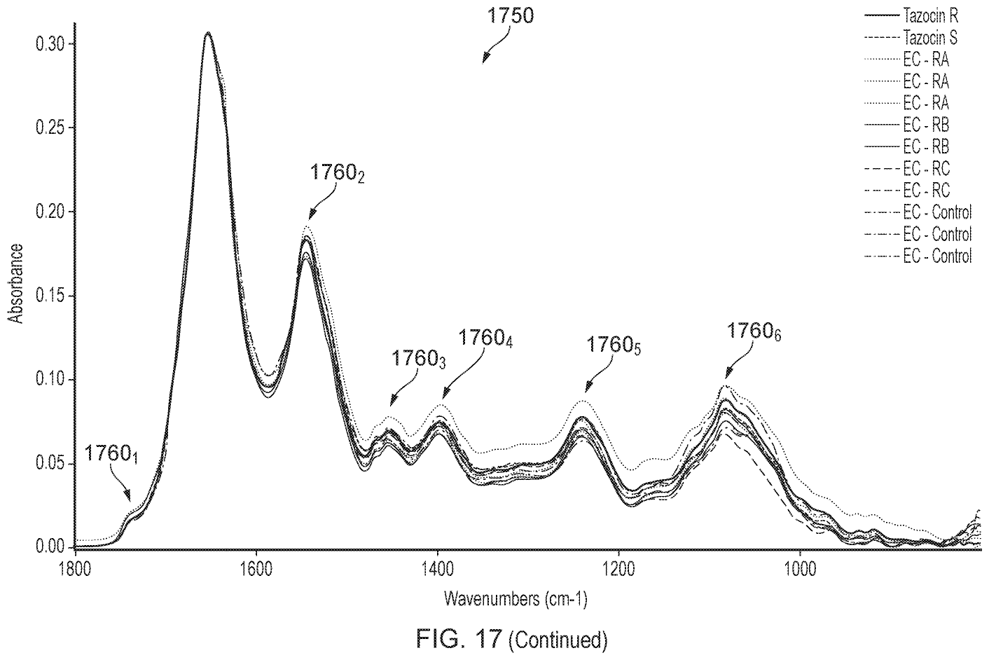

FIG. 17 illustrates a discrimination analysis 1700 and absorbance spectra 1750 for certain gram negative bacteria in a spectral region from 1800-400 cm⁻¹. The discrimination analysis 1700 shows data points associated with Tazocin R 1705, Tazocin S 1710, EC-RA 1715, EC-RB 1720, EC-RB 1725 and EC-Control 1730 as a function of the calculated PC-1, PC-2 and PC-3 (principal components) as described hereinabove. To arrive at discriminant analysis 1700, the data from the spectral region from 1800-400 cm⁻¹ obtained by the vibrational spectroscopy technique is pre-processed, so that unsupervised learning techniques can be used to visualise the data. Discriminant analysis 1700 enables observation of a clear segregation of the data points associated with different microorganisms, thus showing that the clusters of data points are different from each other and allowing data prediction. The plots indicate that a difference in the variance of the data can be obtained from 87% for PC1, 10% for PC2, and 2% for PC3, which allows differentiation of classes. The absorbance spectra 1750 associated with these gram negative bacteria was obtained via FTIR. As can be seen in the spectra 1750, there are a number of peaks 1760₁-1760₆ where differences in the spectra taken from different bacteria are prominent. Differences in the peak shifts 1760₁-1760₅. illustrate amide regions, peak 1760₆ illustrates the differences that occur in the nucleic acid spectral region. By providing spectroscopic data associated with the absorbance spectra 1750 from each antibiotic resistant species (e.g., EC-RA) within this spectral region into one or more machine learning modules, one or more trained machine learning models can be provided as described hereinabove. These trained models are capable of identifying the given antibiotic resistant species for which they have been given training data. Thus, the models are able to identify if the specific antibiotic resistant species is present in an unknown sample when provided with spectra from that sample in this spectral region. The spectra from the Fingerprint region from all the classes associated with spectra 1750 present different shifts in both absorbance and wavenumber and these shifts allow an appropriate differentiation from all classes.

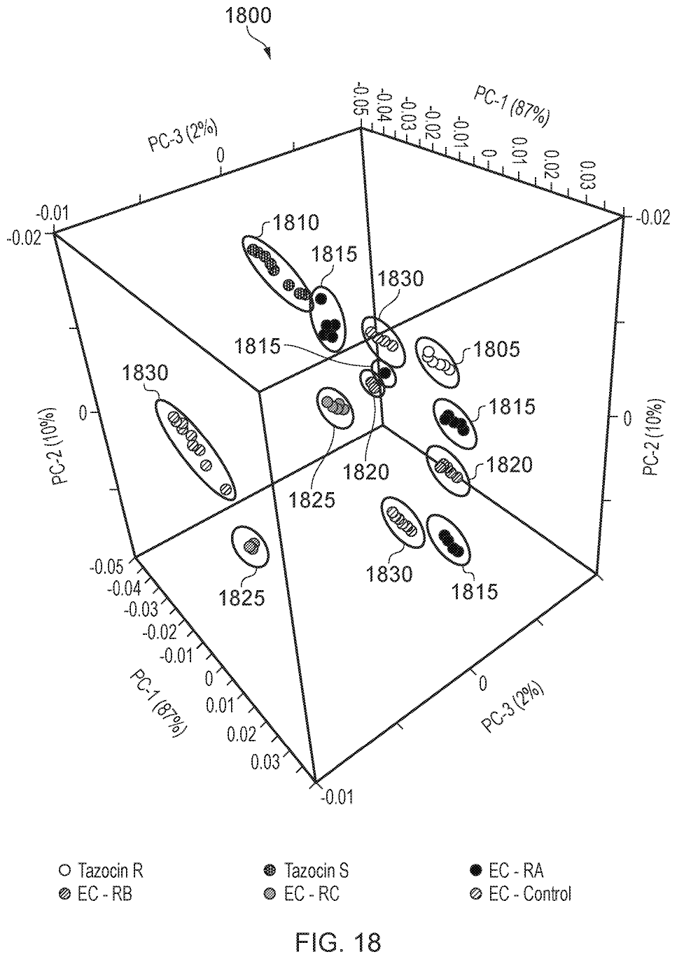

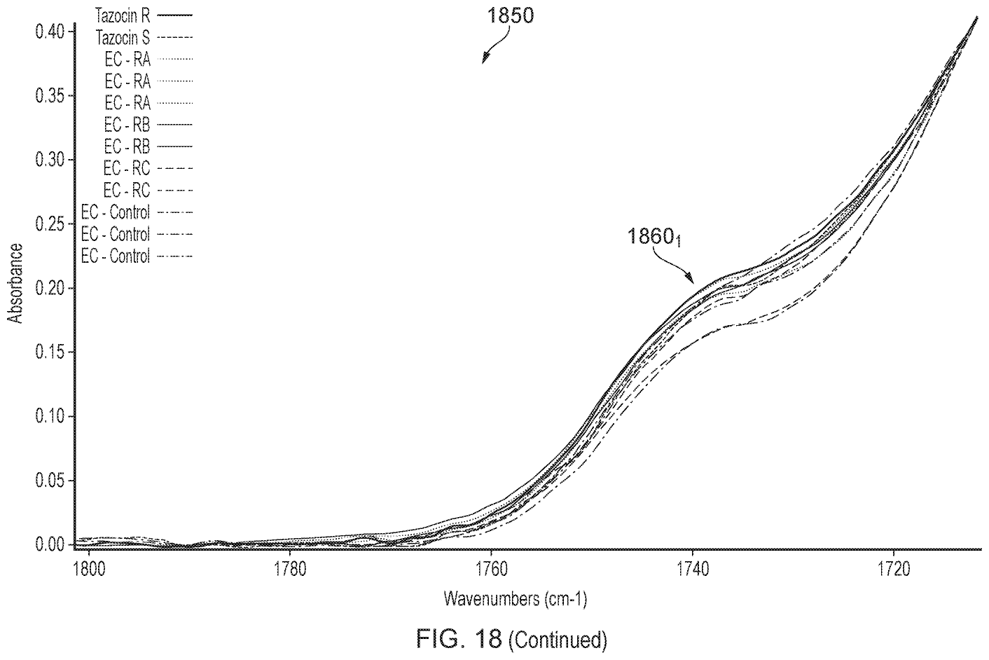

FIG. 18 illustrates a discrimination analysis 1800 and absorbance spectra 1850 for certain gram negative bacteria in a spectral region from 1800-1710 cm⁻¹. The discrimination analysis 1800 shows data points associated with Tazocin R 1805, Tazocin S 1810, EC-RA 1815, EC-RB 1820, EC-RB 1825 and EC-Control 1830 as a function of the calculated PC-1, PC-2 and PC-3 (principal components) as described hereinabove. To arrive at discriminant analysis 1800, the data from the spectral region from 1800-1710 cm⁻¹ obtained by the vibrational spectroscopy technique is pre-processed, so that unsupervised learning techniques can be used to visualise the data. Discriminant analysis 1800 enables observation of a clear segregation of the data points associated with different microorganisms, thus showing that the clusters of data points are different from each other and allowing data prediction. The plots indicate that a difference in the variance of the data can be obtained from 87% for PC1, 10% for PC2, and 2% for PC3, which allows differentiation of classes. The absorbance spectra 1850 associated with these gram negative bacteria was obtained via FTIR. As can be seen in the spectra 1850, there is a peak 1860₁ where differences in the spectra taken from different bacteria are prominent. Differences in the peak shift 1860₁ illustrates the differences that occur on the band assigned to C═O stretching of lipids which varies for each of the gram negative bacteria shown in spectra 1850. By providing spectroscopic data associated with the absorbance spectra 1850 from each antibiotic resistant species (e.g., EC-RA) within this spectral region into one or more machine learning modules, one or more trained machine learning models can be provided as described hereinabove. These trained models are capable of identifying the given antibiotic resistant species for which they have been given training data. Thus, the models are able to identify if the specific antibiotic resistant species is present in an unknown sample when provided with spectra from that sample in this spectral region. The spectra from this fingerprint sub-region from all the classes associated with spectra 1850 present different shifts in both absorbance and wavenumber and these shifts allow an appropriate differentiation from all classes.

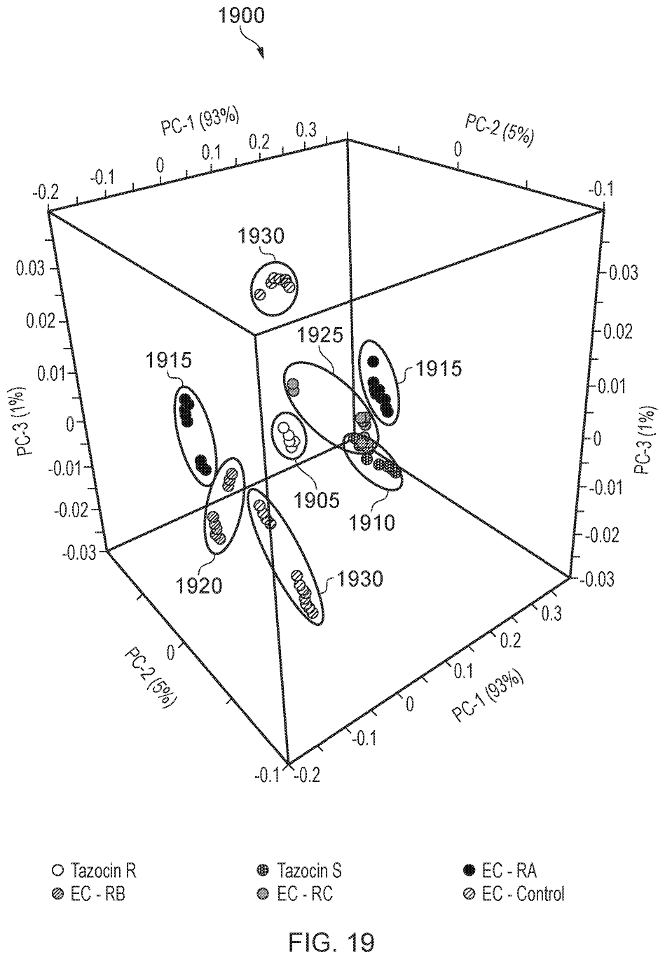

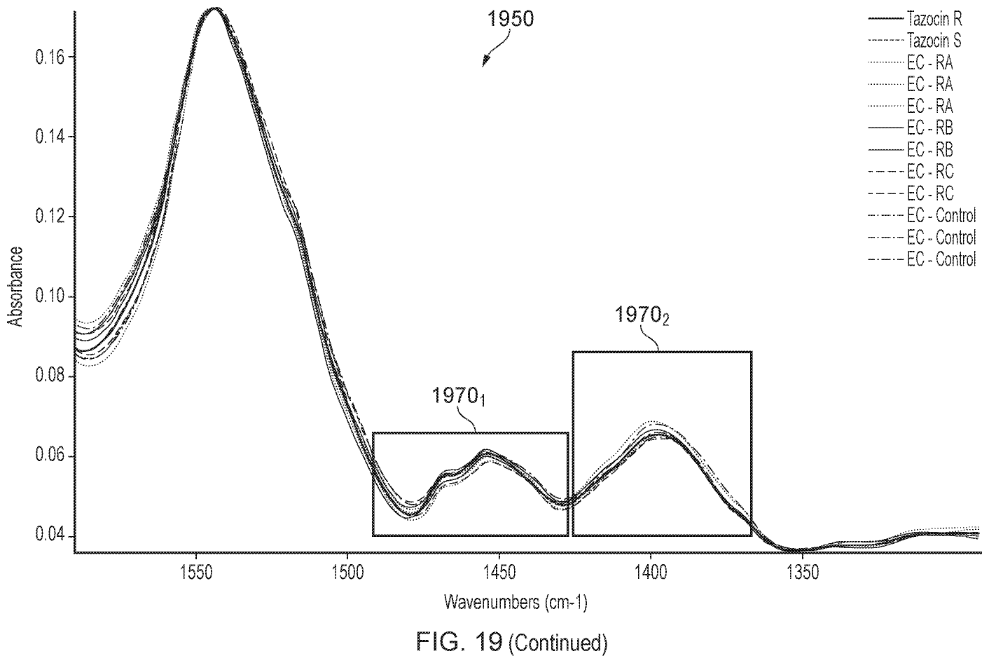

FIG. 19 illustrates a discrimination analysis 1900 and absorbance spectra 1950 for certain gram negative bacteria in a spectral region from 1590-1290 cm⁻¹. The discrimination analysis 1900 shows data points associated with Tazocin R 1905, Tazocin S 1910, EC-RA 1915, EC-RB 1920, EC-RB 1925 and EC-Control 1930 as a function of the calculated PC-1, PC-2 and PC-3 (principal components) as described hereinabove. To arrive at discriminant analysis 1900, the data from the spectral region from 1590-1290 cm⁻¹ obtained by the vibrational spectroscopy technique is pre-processed, so that unsupervised learning techniques can be used to visualise the data. Discriminant analysis 1900 enables observation of a clear segregation of the data points associated with different microorganisms, thus showing that the clusters of data points are different from each other and allowing data prediction. The plots indicate that a difference in the variance of the data can be obtained from 93% for PC1, 5% for PC2, and 1% for PC3, which allows differentiation of classes. The absorbance spectra 1950 associated with these gram negative bacteria was obtained via FTIR. As can be seen in the spectra 1950, there are a number of regions 1970₁, 1970₂ where differences in the spectra taken from different bacteria are prominent. Differences in the peak shifts illustrated in spectra 1950 illustrate different ranges of spectral bands assigned in this fingerprint sub-region, primarily this sub-region includes the amide 2 and 3 spectral bands. A first region 1970₁ shows the differences that occur on the band assigned to CH₃ stretching of lipids and proteins, whereas a second region 1970₂ shows the differences that occur on CH₃ of proteins, an important feature from the amide 3 region which varies for each of the gram negative bacteria shown in spectra 1950. By providing spectroscopic data associated with the absorbance spectra 1950 from each antibiotic resistant species (e.g., EC-RA) within this spectral region into one or more machine learning modules, one or more trained machine learning models can be provided as described hereinabove. These trained models are capable of identifying the given antibiotic resistant species for which they have been given training data. Thus, the models are able to identify if the specific antibiotic resistant species is present in an unknown sample when provided with spectra from that sample in this spectral region. The spectra from this fingerprint sub-region from all the classes associated with spectra 1950 present different shifts in both absorbance and wavenumber and these shifts allow an appropriate differentiation from all classes.

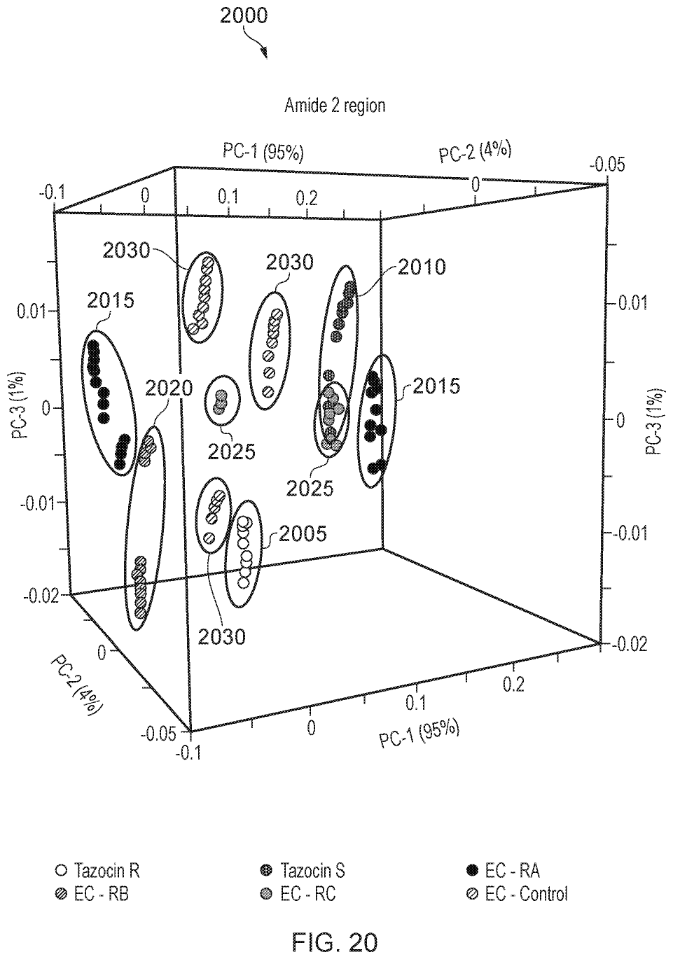

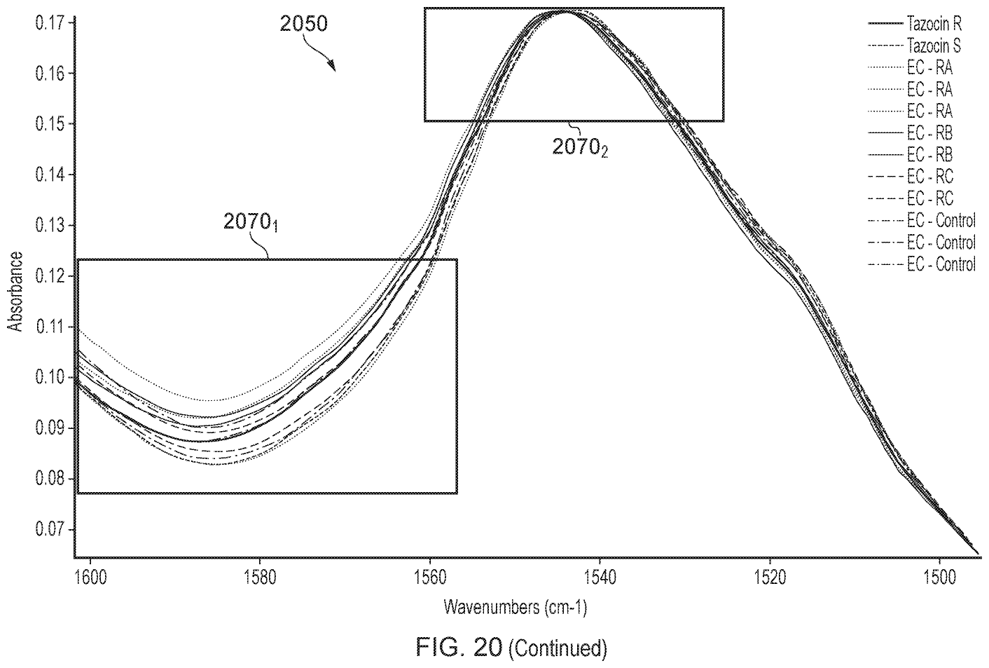

FIG. 20 illustrates a discrimination analysis 2000 and absorbance spectra 2050 for certain gram negative bacteria in a spectral region from 1600-1500 cm⁻¹. The discrimination analysis 2000 shows data points associated with Tazocin R 2005, Tazocin S 2010, EC-RA 2015, EC-RB 2020, EC-RB 2025 and EC-Control 2030 as a function of the calculated PC-1, PC-2 and PC-3 (principal components) as described hereinabove. To arrive at discriminant analysis 2000, the data from the spectral region from 1600-1500 cm⁻¹ obtained by the vibrational spectroscopy technique is pre-processed, so that unsupervised learning techniques can be used to visualise the data. Discriminant analysis 2000 enables observation of a clear segregation of the data points associated with different microorganisms, thus showing that the clusters of data points are different from each other and allowing data prediction. The plots indicate that a difference in the variance of the data can be obtained from 95% for PC1,4% for PC2, and 1% for PC3, which allows differentiation of classes. The absorbance spectra 2050 associated with these gram negative bacteria was obtained via FTIR. As can be seen in the spectra 2050, there are a number of regions 2070₁, 2070₂ where differences in the spectra taken from different bacteria are prominent. Differences in the peak shifts illustrated in spectra 2050. illustrate the spectral differences on each bacteria class. The regions 2070₁ and 2070₂ show the shift differences that occur on the band assigned to amide 2 vibration. By providing spectroscopic data associated with the absorbance spectra 2050 from each antibiotic resistant species (e.g., EC-RA) within this spectral region into one or more machine learning modules, one or more trained machine learning models can be provided as described hereinabove. These trained models are capable of identifying the given antibiotic resistant species for which they have been given training data. Thus, the models are able to identify if the specific antibiotic resistant species is present in an unknown sample when provided with spectra from that sample in this spectral region. The spectra from the fingerprint sub-region from all the classes associated with spectra 2050 present different shifts in both absorbance and wavenumber and these shifts allow an appropriate differentiation from all classes.

As described above, performing a discriminative analysis of the data obtained allows the pre-visualisation of the data to be carried out as a preliminary support for the modelling of machine learning models. The visualisation of clusters in different spectral regions not only allows the feasibility and effectiveness in regions other than the full wavenumber region to be observed, but having identified short regions can also help in the creation of vibrational spectroscopic devices with a lower complexity, both structurally and in terms of size.

Biological Fluid (Urine) Containing Bacteria Differentiation on Species Level

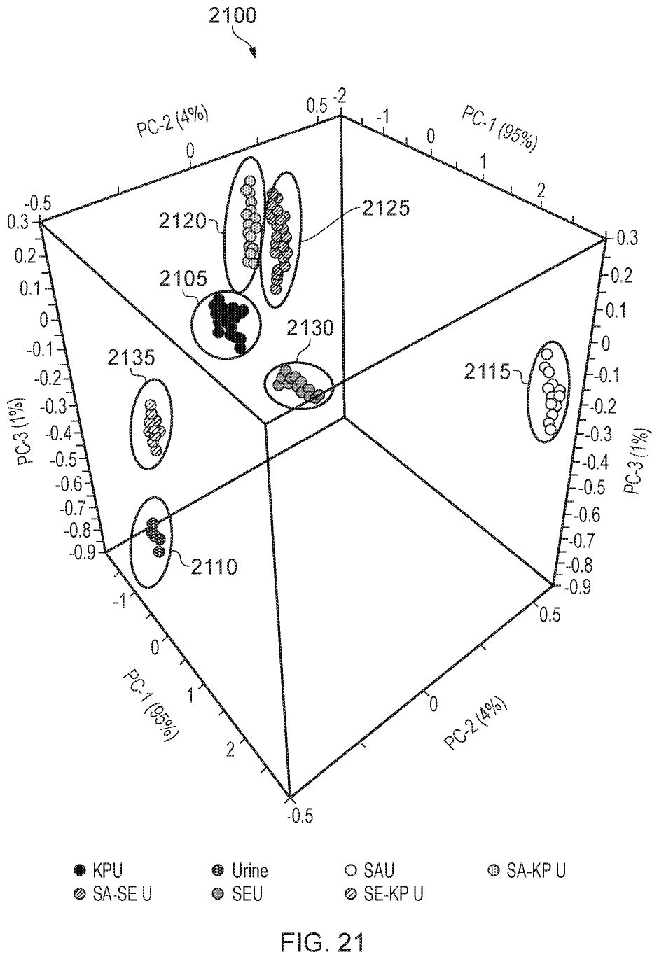

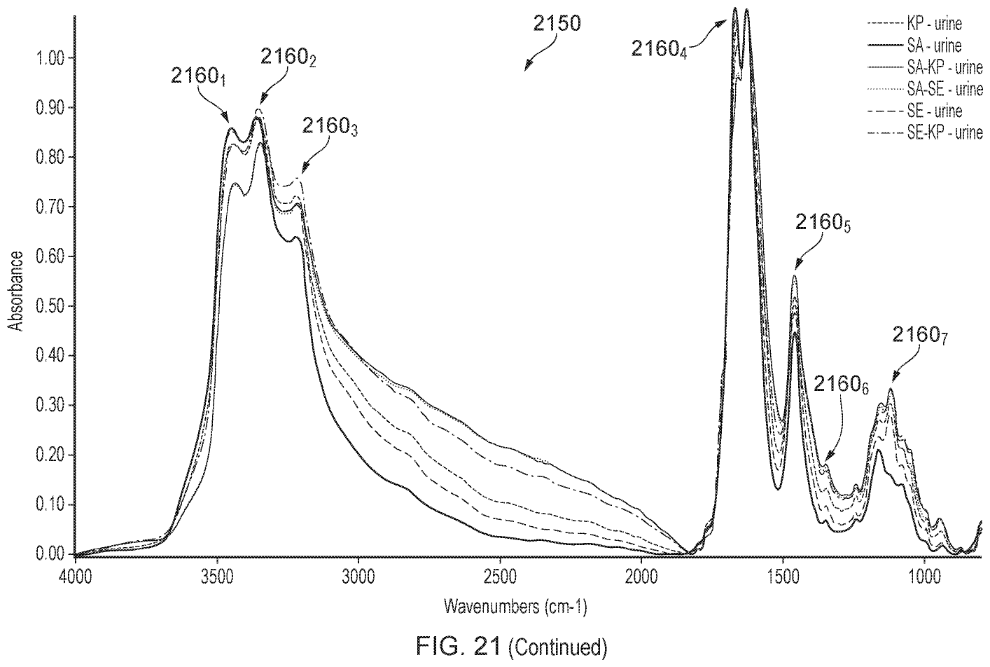

FIGS. 21-25 illustrate data demonstrating speciation of bacteria in direct biofluid (urine) samples. The following examples of bacteria are shown in these figures:
Urine—Staphylococcus aureus (SAU).
Urine—Staphylococcus epidermidis (SEU).
Urine—Klebsiella pneumoniae (KPU).

FIG. 21 illustrates a discrimination analysis 2100 and absorbance spectra 2150 for certain urine-bacteria samples in a spectral region from 4000-400 cm⁻¹. The discrimination analysis 2100 shows data points associated with KPU 2105, Urine 2110, SAU 2115, SA-KP U 2120, SA-SE U 2125, SEU 2130, SE-KP U 2135 as a function of the calculated PC-1, PC-2 and PC-3 (principal components) as described hereinabove. To arrive at discriminant analysis 2100, the data obtained by the vibrational spectroscopy technique is pre-processed, so that unsupervised learning techniques can be used to visualise the data. Discriminant analysis 2100 enables observation of a clear segregation of the data points associated with the different microorganism-urine mixtures, thus showing that the clusters of data points are different from each other and allowing data prediction. The plots indicate that a difference in the variance of the data can be obtained from 95% for PC1, 4% for PC2, and 1% for PC3, which allows differentiation of classes. The absorbance spectra 2150 associated with these urine bacteria samples was obtained via

FTIR. As can be seen in the spectra 2150, there are a number of peaks 2160₁-2160₇ where differences in the spectra taken from different bacteria are prominent. Differences in the peak shifts 2160₁-2160₃ illustrate the differences in the NH₂ region from the urea, peaks 2160₄, 2160₅, 2160₆ illustrate the amide regions, peak 2160₇ illustrates the differences that occur in the nucleic acid spectral region. By providing spectroscopic data associated with the absorbance spectra 2150 from each bacteria species (e.g., KPU) within this spectral region into one or more machine learning modules, one or more trained machine learning models can be provided as described hereinabove. These trained models are capable of identifying the given species for which they have been given training data. Thus, the models are able to identify if the specific species is present in an unknown sample when provided with spectra from that sample in this spectral region. The spectra from all the classes associated with spectra 2150 present different shifts in both absorbance and wavenumber and these shifts allow an appropriate differentiation from all classes.

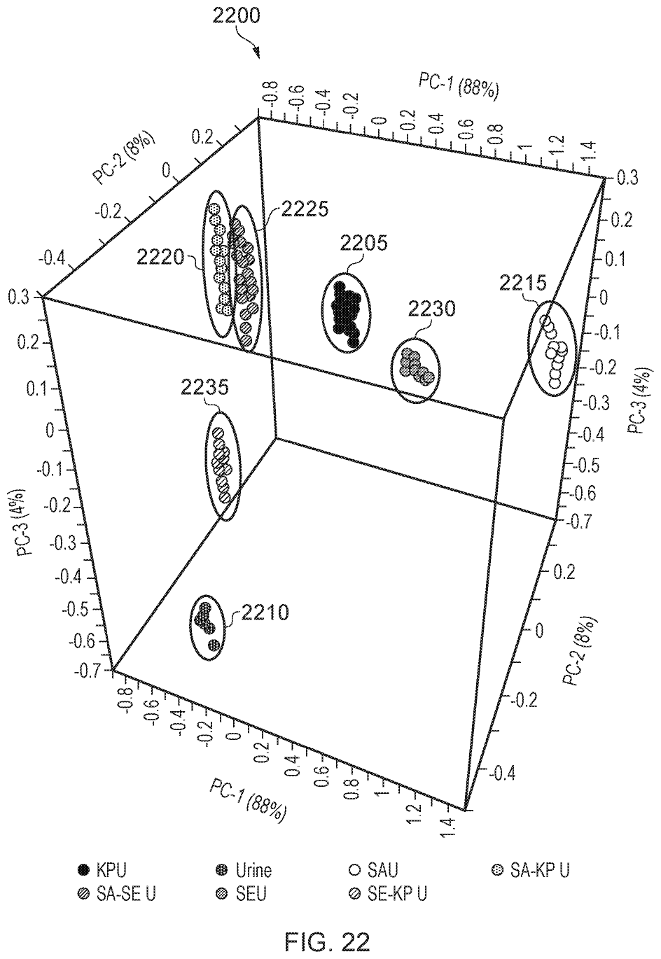

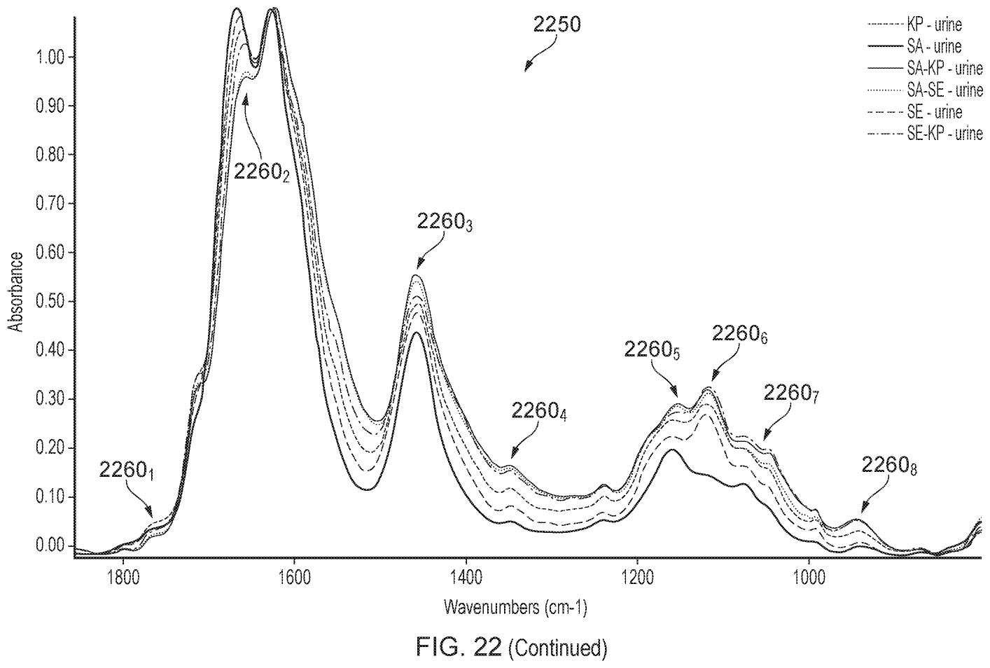

FIG. 22 illustrates a discrimination analysis 2200 and absorbance spectra 2250 for certain urine-bacteria samples in a spectral region from 1800-400 cm⁻¹. The discrimination analysis 2200 shows data points associated with KPU 2205, Urine 2210, SAU 2215, SA-KP U 2220, SA-SE U 2225, SEU 2230, SE-KP U 2235 as a function of the calculated PC-1, PC-2 and PC-3 (principal components) as described hereinabove. To arrive at discriminant analysis 2200, the data from the spectral region from 1800-400 cm⁻¹ obtained by the vibrational spectroscopy technique is pre-processed, so that unsupervised learning techniques can be used to visualise the data. Discriminant analysis 2200 enables observation of a clear segregation of the data points associated with different microorganism-urine mixtures, thus showing that the clusters of data points are different from each other and allowing data prediction. The plots indicate that a difference in the variance of the data can be obtained from 88% for PC1, 8% for PC2, and 4% for PC3, which allows differentiation of classes. The absorbance spectra 2250 associated with these urine bacteria samples was obtained via FTIR. As can be seen in the spectra 2250, there are a number of peaks 2260₁-2260₈ where differences in the spectra taken from different bacteria are prominent. Differences in the peak shifts 2260₁-2260₄ illustrate amide regions, peaks 2260₅-2260₈ illustrate the differences that occur in the nucleic acid spectral region. By providing spectroscopic data associated with the absorbance spectra 2250 from each bacteria species (e.g., KPU) within this spectral region into one or more machine learning modules, one or more trained machine learning models can be provided as described hereinabove. These trained models are capable of identifying the given species for which they have been given training data. Thus, the models are able to identify if the specific species is present in an unknown sample when provided with spectra from that sample in this spectral region. The spectra from the Fingerprint region from all the classes associated with spectra 2250 present different shifts in both absorbance and wavenumber and these shifts allow an appropriate differentiation from all classes.

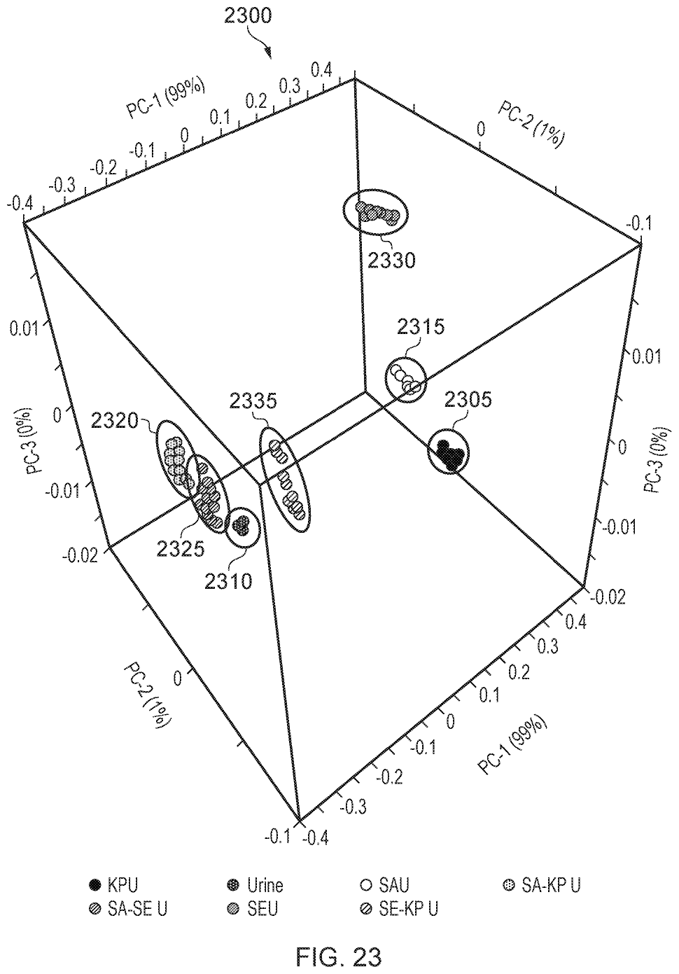

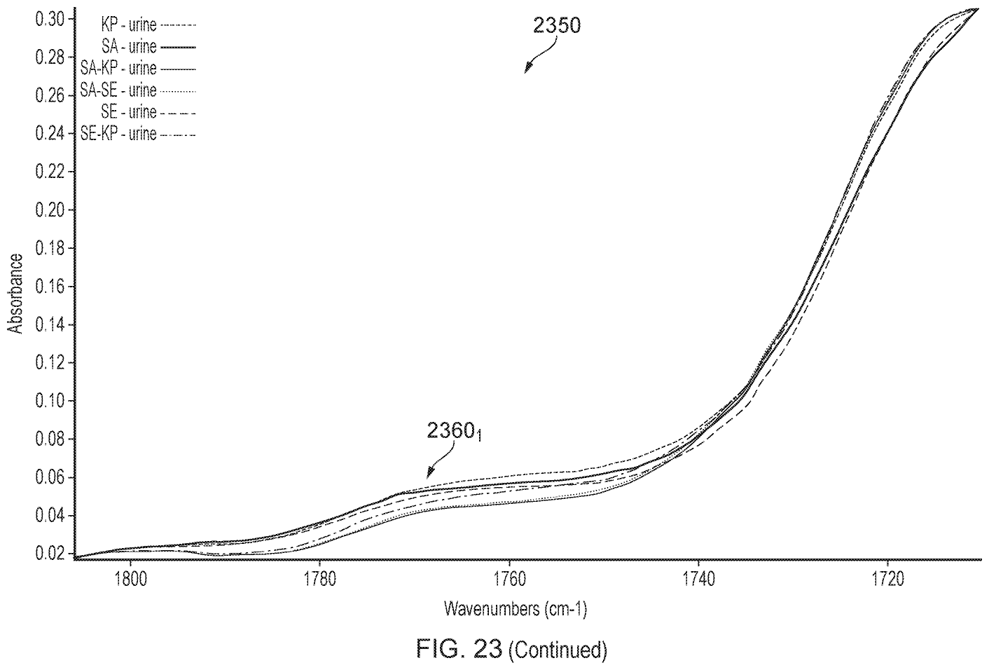

FIG. 23 illustrates a discrimination analysis 2300 and absorbance spectra 2350 for certain urine-bacteria samples in a spectral region from 1800-1710 cm⁻¹. The discrimination analysis 2300 shows data points associated with KPU 2305, Urine 2310, SAU 2315, SA-KP U 2320, SA-SE U 2325, SEU 2330, SE-KP U 2335 as a function of the calculated PC-1, PC-2 and PC-3 (principal components) as described hereinabove. To arrive at discriminant analysis 2300, the data from the spectral region from 1800-1710 cm⁻¹ obtained by the vibrational spectroscopy technique is pre-processed, so that unsupervised learning techniques can be used to visualise the data. Discriminant analysis 2300 enables observation of a clear segregation of the data points associated with different microorganism-urine mixtures, thus showing that the clusters of data points are different from each other and allowing data prediction. The plots indicate that a difference in the variance of the data can be obtained from 99% for PC1, 1% for PC2, and 0% for PC3, which allows differentiation of classes. The absorbance spectra 2350 associated with these urine bacteria samples was obtained via FTIR. As can be seen in the spectra 2350, there is a peak 2360₁ where differences in the spectra taken from different bacteria are prominent. Differences in the peak shift 2360₁ illustrates the differences that occur on the band assigned to C═O stretching of lipids which varies for each bacteria class shown in spectra 2350. By providing spectroscopic data associated with the absorbance spectra 2350 from each bacteria species (e.g., KPU) within this spectral region into one or more machine learning modules, one or more trained machine learning models can be provided as described hereinabove. These trained models are capable of identifying the given species for which they have been given training data. Thus, the models are able to identify if the specific species is present in an unknown sample when provided with spectra from that sample in this spectral region. The spectra from this fingerprint sub-region from all the classes associated with spectra 2350 present different shifts in both absorbance and wavenumber and these shifts allow an appropriate differentiation from all classes.

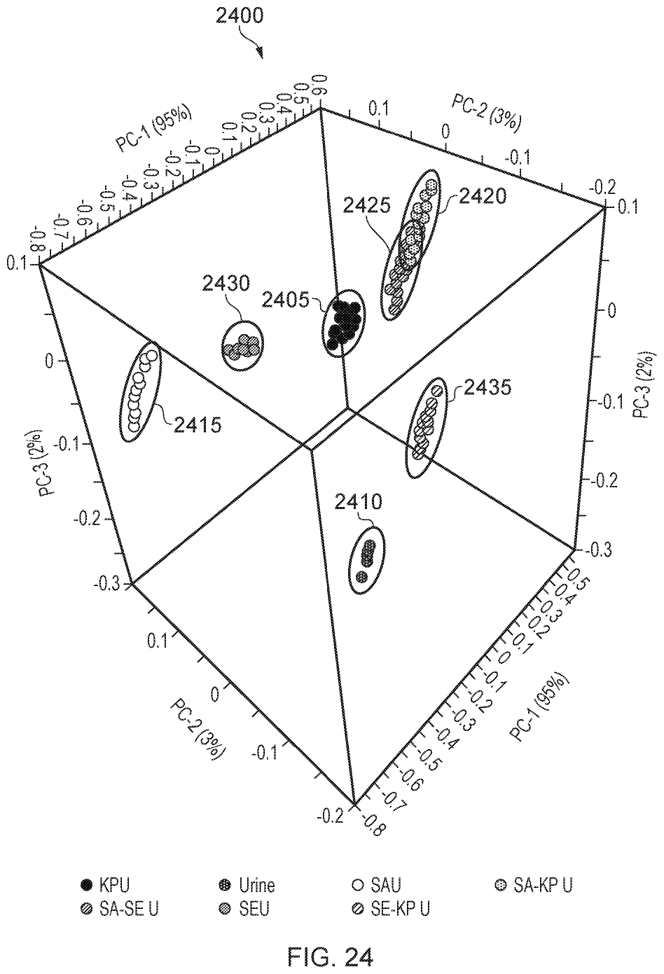

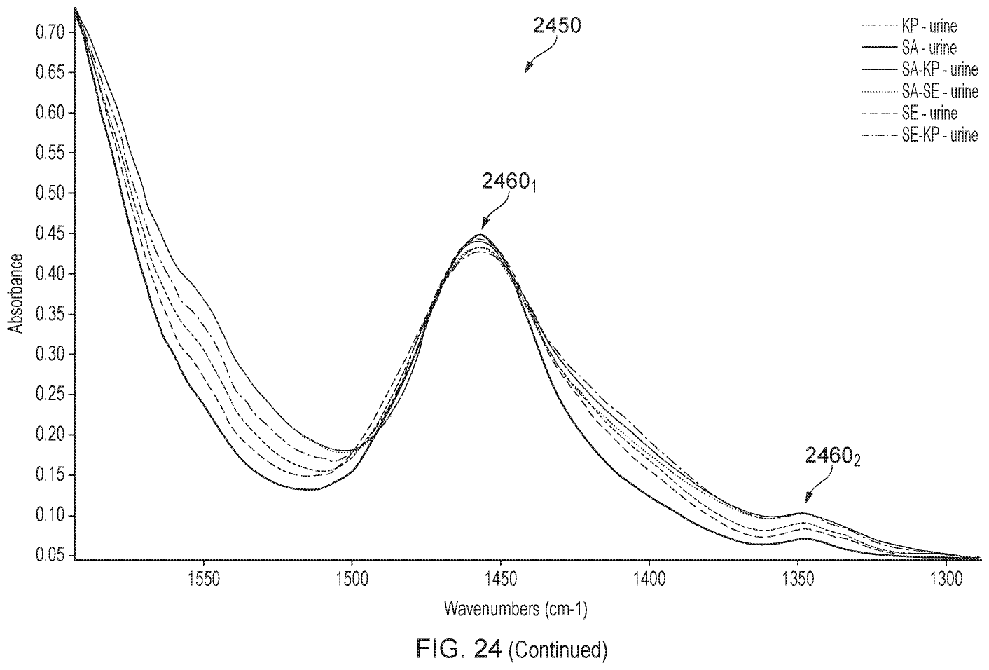

FIG. 24 illustrates a discrimination analysis 2400 and absorbance spectra 2450 for certain urine-bacteria samples in a spectral region from 1590-1290 cm⁻¹. The discrimination analysis 2400 shows data points associated with KPU 2405, Urine 2410, SAU 2415, SA-KP U 2420, SA-SE U 2425, SEU 2430, SE-KP U 2435 as a function of the calculated PC-1, PC-2 and PC-3 (principal components) as described hereinabove. To arrive at discriminant analysis 2400, the data from the spectral region from 1590-1290 cm⁻¹ obtained by the vibrational spectroscopy technique is pre-processed, so that unsupervised learning techniques can be used to visualise the data.

Discriminant analysis 2400 enables observation of a clear segregation of the data points associated with the different microorganism-urine mixtures, thus showing that the clusters of data points are different from each other and allowing data prediction. The plots indicate that a difference in the variance of the data can be obtained from 95% for PC1, 3% for PC2, and 2% for PC3, which allows differentiation of classes. The absorbance spectra 2450 associated with these urine bacteria samples was obtained via FTIR. As can be seen in the spectra 2450, there are a number of peaks 2460₁-2460₂ where differences in the spectra taken from different bacteria are prominent. Differences in the peak shifts illustrated in spectra 2450 illustrate different ranges of spectral bands assigned in this fingerprint sub-region, primarily this sub-region includes the amide 2 and 3 spectral bands. Peak 2460₁ shows the differences that occur on the band assigned to CH₃ stretching of lipids and proteins, whereas peak 2460₂ shows the differences that occur on CH₃ of proteins, an important feature from the amide 3 region which varies for each bacteria class sown in spectra 2450. By providing spectroscopic data associated with the absorbance spectra 2450 from each bacteria species (e.g., KPU) within this spectral region into one or more machine learning modules, one or more trained machine learning models can be provided as described hereinabove. These trained models are capable of identifying the given species for which they have been given training data. Thus, the models are able to identify if the specific species is present in an unknown sample when provided with spectra from that sample in this spectral region. The spectra from this fingerprint sub-region from all the classes associated with spectra 2450 present different shifts in both absorbance and wavenumber and these shifts allow an appropriate differentiation from all classes.

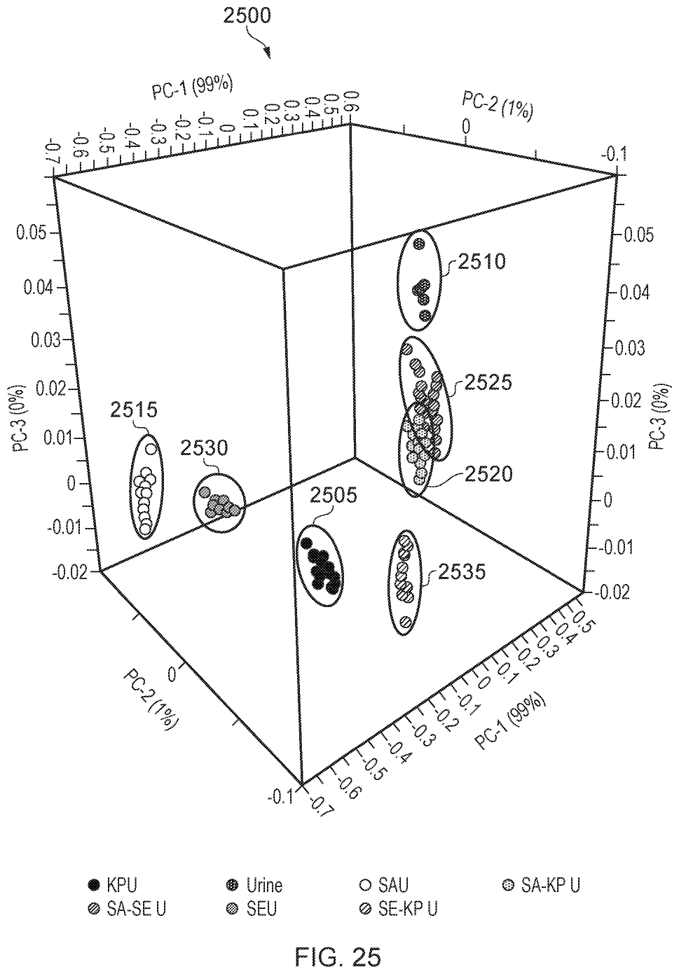

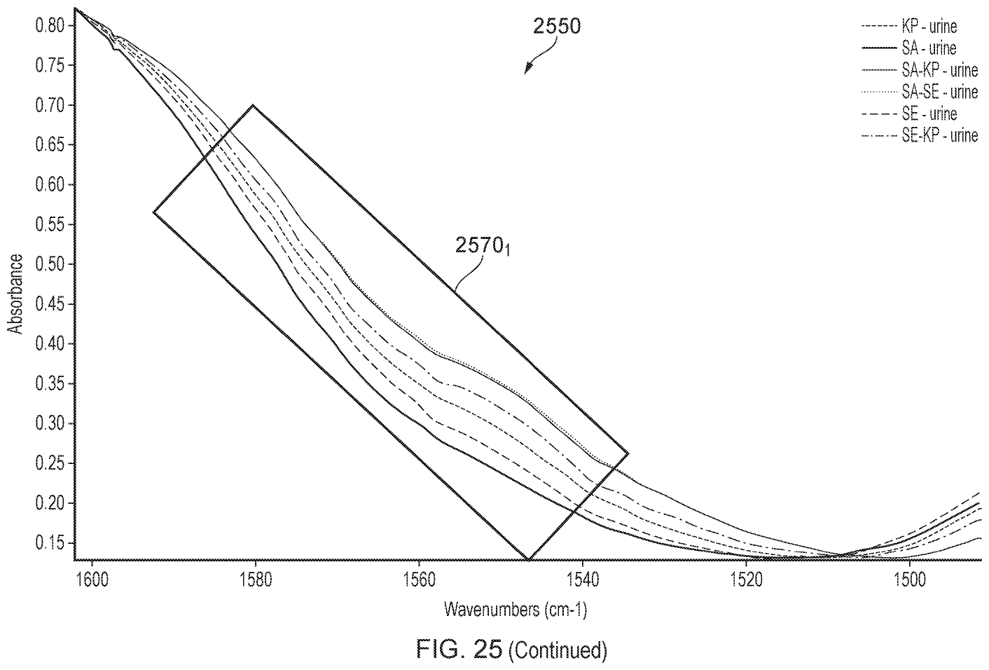

FIG. 25 illustrates a discrimination analysis 2500 and absorbance spectra 2550 for certain urine-bacteria samples in a spectral region from 1600-1500 cm⁻¹. The discrimination analysis 2500 shows data points associated with KPU 2505, Urine 2510, SAU 2515, SA-KP U 2520, SA-SE U 2525, SEU 2530, SE-KP U 2535 as a function of the calculated PC-1, PC-2 and PC-3 (principal components) as described hereinabove. To arrive at discriminant analysis 2500, the data from the spectral region from 1600-1500 cm⁻¹ obtained by the vibrational spectroscopy technique is pre-processed, so that unsupervised learning techniques can be used to visualise the data. Discriminant analysis 2500 enables observation of a clear segregation of the data points associated with different microorganism-urine mixtures, thus showing that the clusters of data points are different from each other and allowing data prediction. The plots indicate that a difference in the variance of the data can be obtained from 99% for PC1, 1% for PC2, and 0% for PC3, which allows differentiation of classes. The absorbance spectra 2550 associated with these urine bacteria samples was obtained via FTIR. As can be seen in the spectra 2250, there is a peak 2560₁ where differences in the spectra taken from different bacteria are prominent. Differences in the peak shifts illustrated in spectra 2550 illustrate the spectral differences on each bacteria-urine class. Peak 2560₁ shows the shift differences that occur on the amide 2 band region. By providing spectroscopic data associated with the absorbance spectra 2550 from each bacteria species (e.g., KPU) within this spectral region into one or more machine learning modules, one or more trained machine learning models can be provided as described hereinabove. These trained models are capable of identifying the given species for which they have been given training data. Thus, the models are able to identify if the specific species is present in an unknown sample when provided with spectra from that sample in this spectral region. The spectra from this fingerprint sub-region from all the classes associated with spectra 2550 present different shifts in both absorbance and wavenumber and these shifts allow an appropriate differentiation from all classes.

As described above, performing a discriminative analysis of the data obtained allows the pre-visualisation of the data to be carried out as a preliminary support for the modelling of machine learning models. The visualisation of clusters in different spectral regions not only allows the feasibility and effectiveness in regions other than the full wavenumber region to be observed, but having identified short regions can also help in the creation of vibrational spectroscopic devices with a lower complexity, both structurally and in terms of size.

Biological Fluid (Saliva) Containing SARS-COV-2 Virus

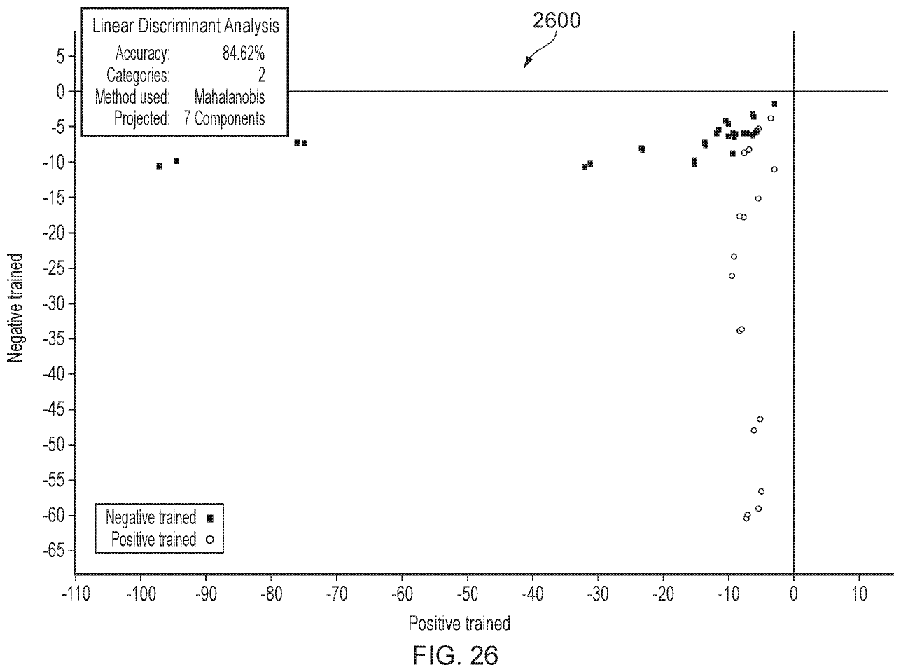

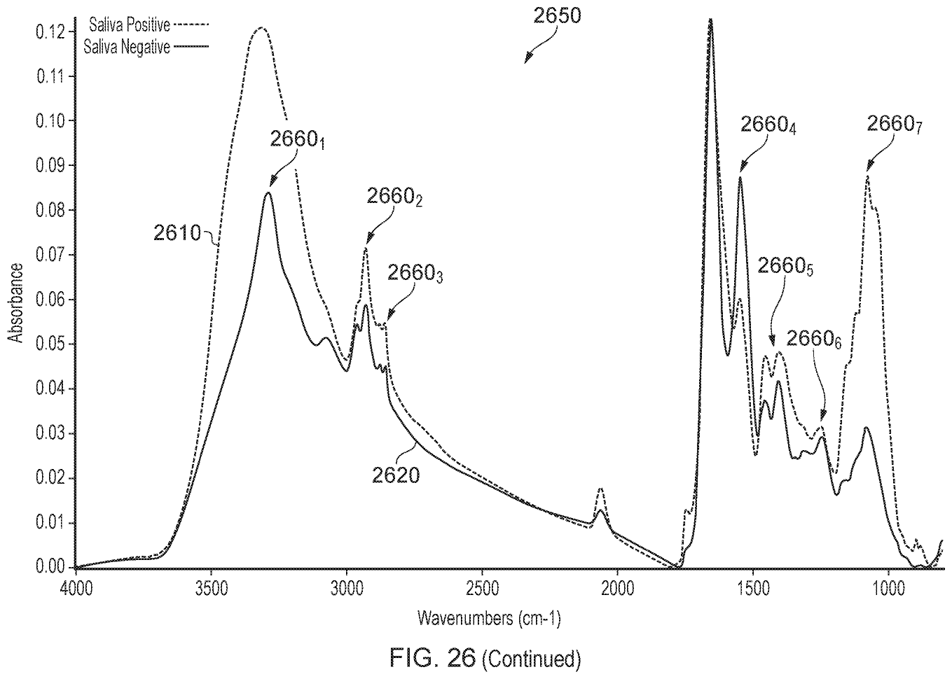

FIGS. 26-29 illustrate data demonstrating detection of SARS-COV-2 virus from direct biofluid (saliva) samples. Whilst certain spectral regions are shown in the following examples, the following spectral regions are useful for virus analysis:
4000-400 cm⁻¹ (‘full wavenumber’ region)
4000-2500 cm⁻¹ (‘high wavenumber’ region)
3800-2500 cm⁻¹
1800-400 cm⁻¹ (‘fingerprint’ region)
1810-1700 cm⁻¹
1590-1290 cm⁻¹
1700-1600 cm⁻¹ (‘Amide 1’ region)
1600-1500 cm⁻¹ (‘Amide 2’ region)
1200-1000 cm⁻¹
1000-400 cm⁻¹
1500-1200 cm⁻¹

FIG. 26 illustrates a discrimination analysis 2600 and absorbance spectra 2650 for COVID positive 2610 and COVID negative 2620 saliva samples of patients in a spectral region from 4000-400 cm⁻¹. The discrimination analysis 2600 shows data points associated with COVID positive saliva samples (red dots) and COVID negative saliva samples (blue squares). To arrive at discriminant analysis 2600, the data obtained by the vibrational spectroscopy technique in the spectral region from 4000-400 cm⁻¹ is pre-processed, so that unsupervised coupled with supervised learning techniques (PCA-LDA) can be used to visualise the data. Discriminant analysis 2600 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 2650 associated with these saliva samples was obtained via FTIR. As can be seen in the spectra 2650, there are a number of peaks 2660₁-2660₇ where differences in the spectra taken from COVID positive/negative samples are prominent. Differences in the peak shifts 2660₁-2660₃ illustrate the differences in the lipid region, peaks 2660₄-2660₆ illustrate amide regions, peak 2660₇ illustrates the differences that occur in the nucleic acid spectral region. By providing spectroscopic data associated with the absorbance spectra 2650 from COVID positive/negative saliva samples within this spectral region into one or more machine learning modules, one or more trained machine learning models can be provided as described hereinabove. These trained models are able to identify if a saliva sample is COVID positive or negative when provided with spectra from that sample in this spectral region. The spectra from all the classes associated with spectra 2650 present different shifts in both absorbance and wavenumber and these shifts allow an appropriate differentiation from all classes.

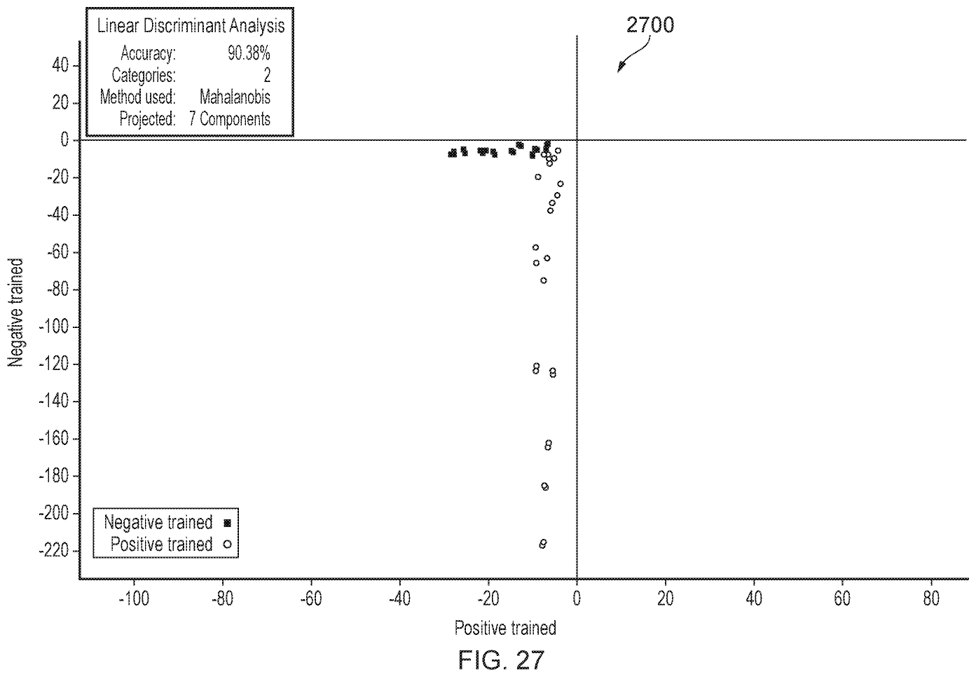

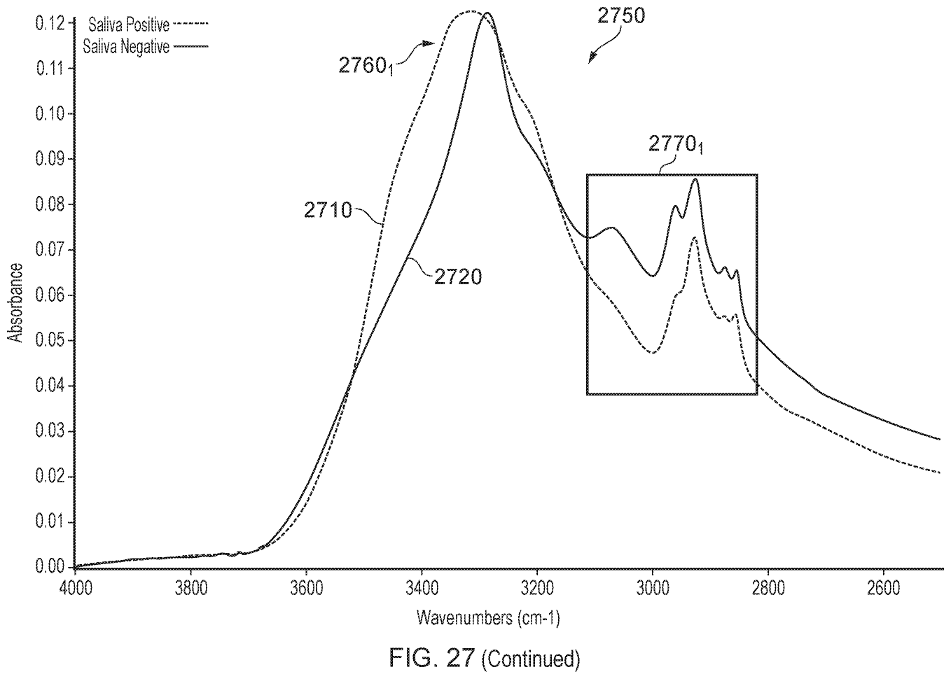

FIG. 27 illustrates a discrimination analysis 2700 and absorbance spectra 2750 for COVID positive 2710 and COVID negative 2720 saliva samples of patients in a spectral region from 4000-2500 cm⁻¹. The discrimination analysis 2700 shows data points associated with COVID positive saliva samples (red dots) and COVID negative saliva samples (blue squares). To arrive at discriminant analysis 2700, the data obtained by the vibrational spectroscopy technique in the spectral region from 4000-2500 cm⁻¹ is pre-processed, so that unsupervised coupled with supervised learning techniques (PCA-LDA) can be used to visualise the data. Discriminant analysis 2700 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 2750 associated with these saliva samples was obtained via FTIR. As can be seen in the spectra 2750, there is a peak 2760₁ and a region 2770₁ where differences in the spectra taken from COVID positive/negative samples are prominent. Differences in the peak shift 2760₁ and in region 2770₁ illustrates differences in CH vibrations from lipids and proteins between spectra 2710 and 2720. By providing spectroscopic data associated with the absorbance spectra 2750 from COVID positive/negative saliva samples within this spectral region into one or more machine learning modules, one or more trained machine learning models can be provided as described hereinabove. These trained models are able to identify if a saliva sample is COVID positive or negative when provided with spectra from that sample in this spectral region. The spectra from all the classes associated with spectra 2750 present different shifts in both absorbance and wavenumber and these shifts allow an appropriate differentiation from all classes.

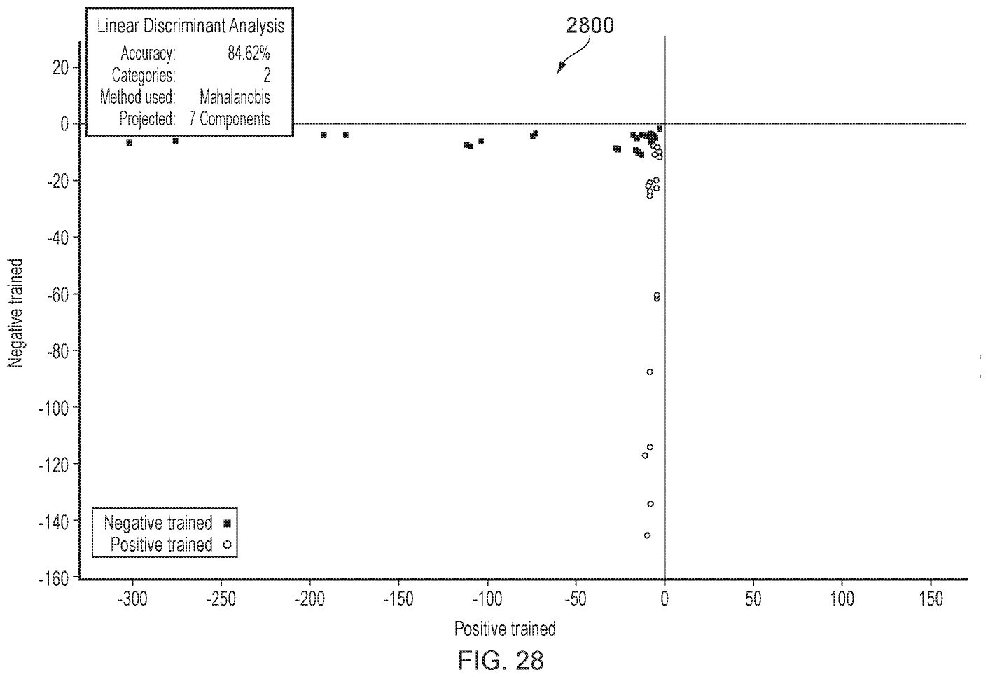

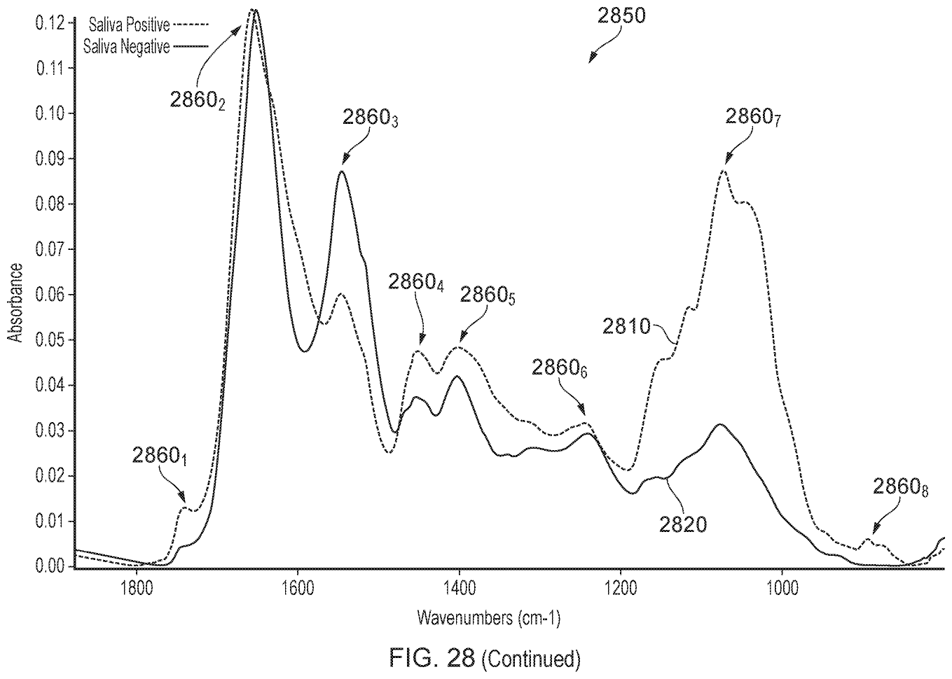

FIG. 28 illustrates a discrimination analysis 2800 and absorbance spectra 2850 for COVID positive 2810 and COVID negative 2820 saliva samples of patients in a spectral region from 1800-400 cm⁻¹. The discrimination analysis 2800 shows data points associated with COVID positive saliva samples (red dots) and COVID negative saliva samples (blue squares). To arrive at discriminant analysis 2800, the data obtained by the vibrational spectroscopy technique in the spectral region from 1800-400 cm⁻¹ is pre-processed, so that unsupervised coupled with supervised learning techniques (PCA-LDA) can be used to visualise the data. Discriminant analysis 2800 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 2850 associated with these saliva samples was obtained via FTIR. As can be seen in the spectra 2850, there are a number of peaks 2860₁-2860₈ where differences in the spectra taken from COVID positive/negative samples are prominent. Differences between spectra 2810 and 2820 in the peak shifts 2860₁-2860₆ illustrate amide regions, peaks 2860₇-2860₈ illustrate the differences that occur in the nucleic acid spectral region. By providing spectroscopic data associated with the absorbance spectra 2850 from COVID positive/negative saliva samples within this spectral region into one or more machine learning modules, one or more trained machine learning models can be provided as described hereinabove. These trained models are able to identify if a saliva sample is COVID positive or negative when provided with spectra from that sample in this spectral region. The spectra from all the classes associated with spectra 2850 present different shifts in both absorbance and wavenumber and these shifts allow an appropriate differentiation from all classes.

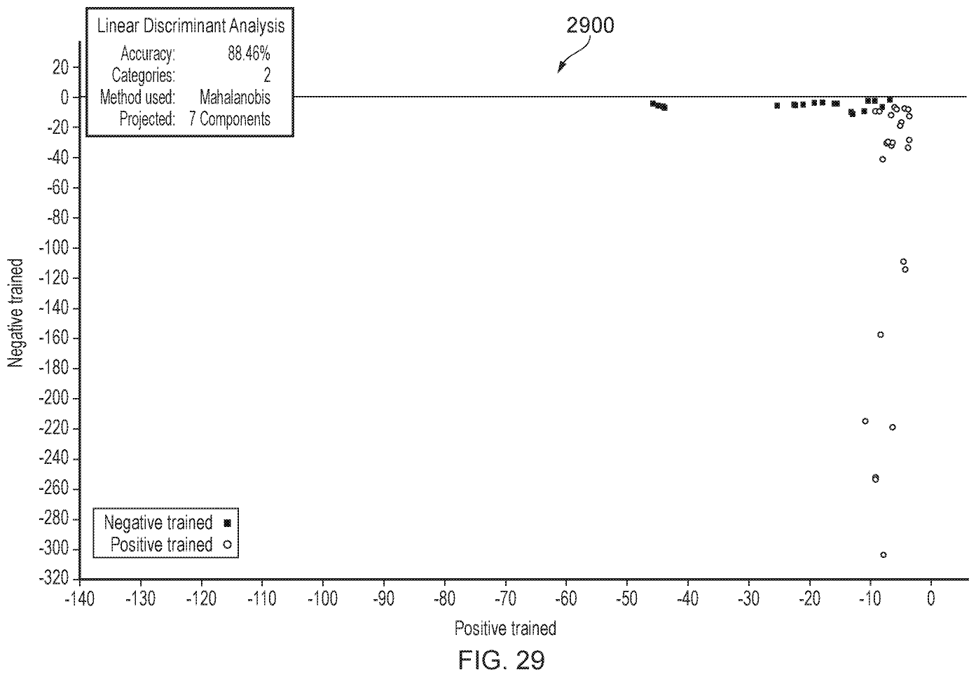

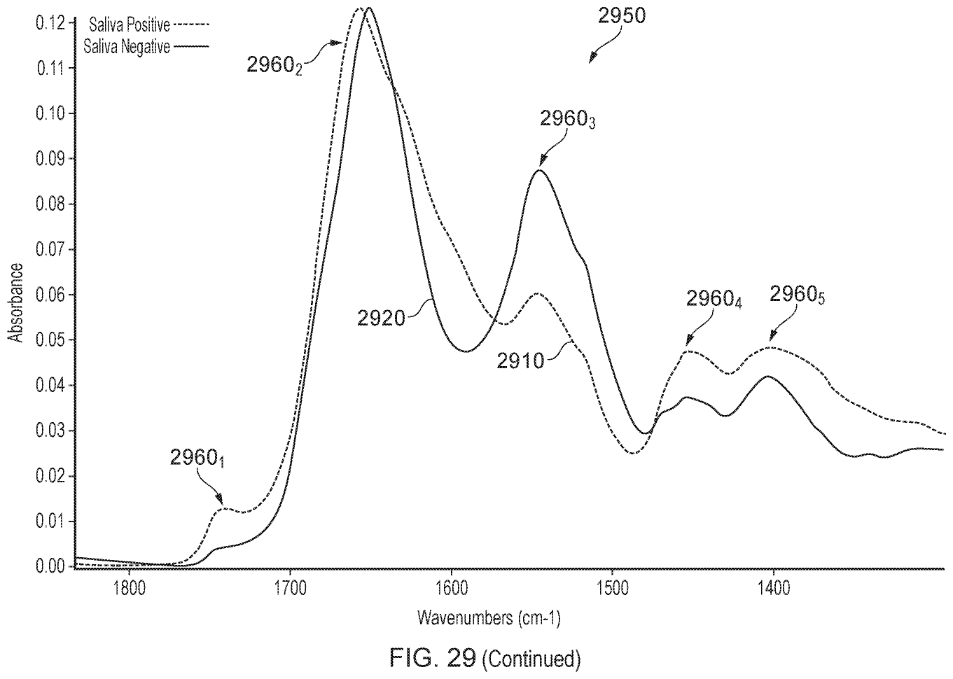

FIG. 29 illustrates a discrimination analysis 2900 and absorbance spectra 2950 for COVID positive 2910 and COVID negative 2920 saliva samples of patients in a spectral region from 1800-1300 cm⁻¹. The discrimination analysis 2900 shows data points associated with COVID positive saliva samples (red dots) and COVID negative saliva samples (blue squares). To arrive at discriminant analysis 2900, the data obtained by the vibrational spectroscopy technique in the spectral region from 1800-1300 cm⁻¹ is pre-processed, so that unsupervised coupled with supervised learning techniques (PCA-LDA) can be used to visualise the data. Discriminant analysis 2900 enables observation of a clear segregation of the data points associate with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 2950 associated with these saliva samples was obtained via FTIR. As can be seen in the spectra 2950, there are a number of peaks 2960₁-2960₅ where differences in the spectra taken from COVID positive/negative samples are prominent. Differences between spectra 2910 and 2920 in the peak shifts 2960₁-2960₅ illustrate amide regions. By providing spectroscopic data associated with the absorbance spectra 2950 from COVID positive/negative saliva samples within this spectral region into one or more machine learning modules, one or more trained machine learning models can be provided as described hereinabove. These trained models are able to identify if a saliva sample is COVID positive or negative when provided with spectra from that sample in this spectral region. The spectra from all the classes associated with spectra 2950 present different shifts in both absorbance and wavenumber and these shifts allow an appropriate differentiation from all classes.

As described above, performing a discriminative analysis of the data obtained allows the pre-visualisation of the data to be carried out as a preliminary support for the modelling of machine learning models. The visualisation of clusters in different spectral regions not only allows the feasibility and effectiveness in regions other than the full wavenumber region to be observed, but having identified short regions can also help in the creation of vibrational spectroscopic devices with a lower complexity, both structurally and in terms of size.

Biological Fluid (Nasopharyngeal) Containing SARS-COV-2 Virus

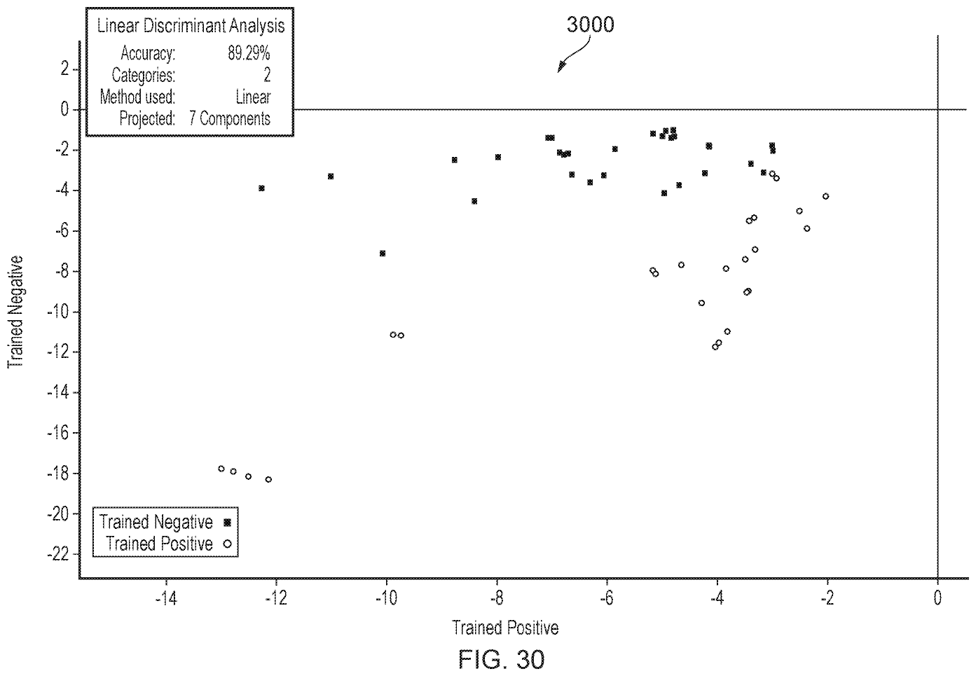

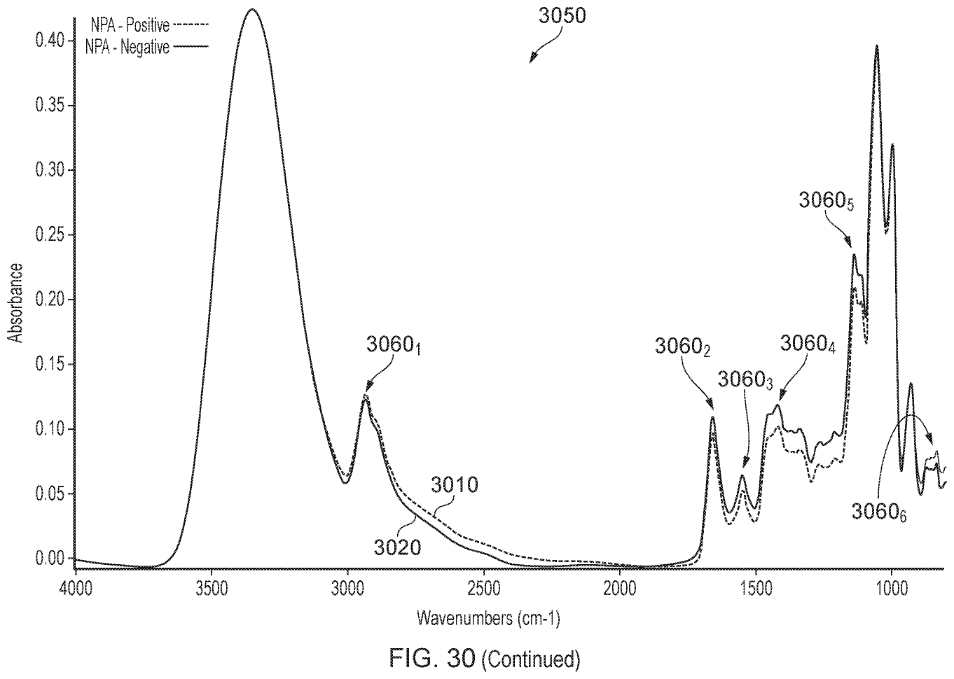

FIGS. 30-33 illustrate data demonstrating detection of SARS-COV-2 virus from direct biofluid (nasopharyngeal) samples. Whilst certain spectral regions are shown in the following examples, the following spectral regions are useful for virus analysis:
4000-400 cm⁻¹ (‘full wavenumber’ region)
4000-2500 cm⁻¹ (‘high wavenumber’ region)
3800-2500 cm 1
1800-400 cm⁻¹ (‘fingerprint’ region)
1810-1700 cm⁻¹
1590-1290 cm⁻¹
1700-1600 cm⁻¹ (‘Amide 1’ region)
1600-1500 cm⁻¹ (‘Amide 2’ region)
1200-1000 cm⁻¹
1000-400 cm⁻¹
1500-1200 cm⁻¹

FIG. 30 illustrates a discrimination analysis 3000 and absorbance spectra 3050 for COVID positive 3010 and COVID negative 3020 nasopharyngeal samples of patients in a spectral region from 4000-400 cm⁻¹. The discrimination analysis 3000 shows data points associated with COVID positive nasopharyngeal samples (red dots) and COVID negative nasopharyngeal samples (blue squares). To arrive at discriminant analysis 3000, the data obtained by the vibrational spectroscopy technique in the spectral region from 4000-400 cm⁻¹ is pre-processed, so that unsupervised coupled with supervised learning techniques (PCA-LDA) can be used to visualise the data. Discriminant analysis 3000 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 3050 associated with these nasopharyngeal samples was obtained via FTIR. As can be seen in the spectra 3050, there are a number of peaks 3060₁-3060₆ where differences in the spectra taken from COVID positive/negative samples are prominent. Differences in the peak shift 3060₁ illustrates the differences in CH vibrations, peaks 3060₂-3060₄ illustrate amide regions, peak 3060₅ illustrates the differences that occur on CH₂, CO, and/or CC vibrations of polysaccharides, and peak 3060₆ illustrates the C₂ vibration from nucleic acids. By providing spectroscopic data associated with the absorbance spectra 3050 from COVID positive/negative nasopharyngeal samples within this spectral region into one or more machine learning modules, one or more trained machine learning models can be provided as described hereinabove. These trained models are able to identify if a nasopharyngeal sample is COVID positive or negative when provided with spectra from that sample in this spectral region. The spectra from all the classes associated with spectra 3050 present different shifts in both absorbance and wavenumber and these shifts allow an appropriate differentiation from all classes.

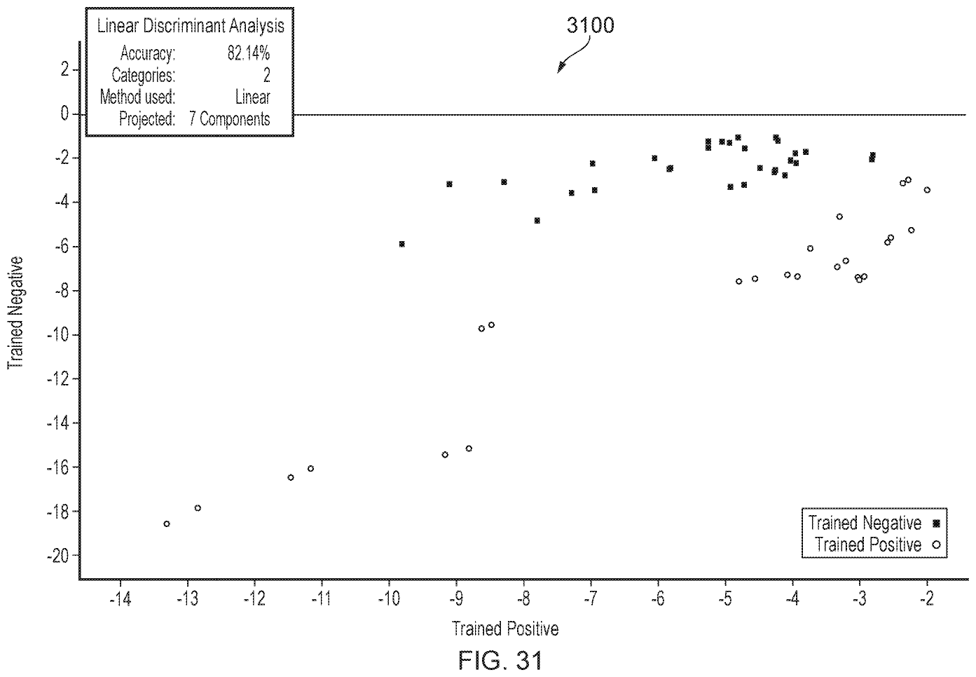

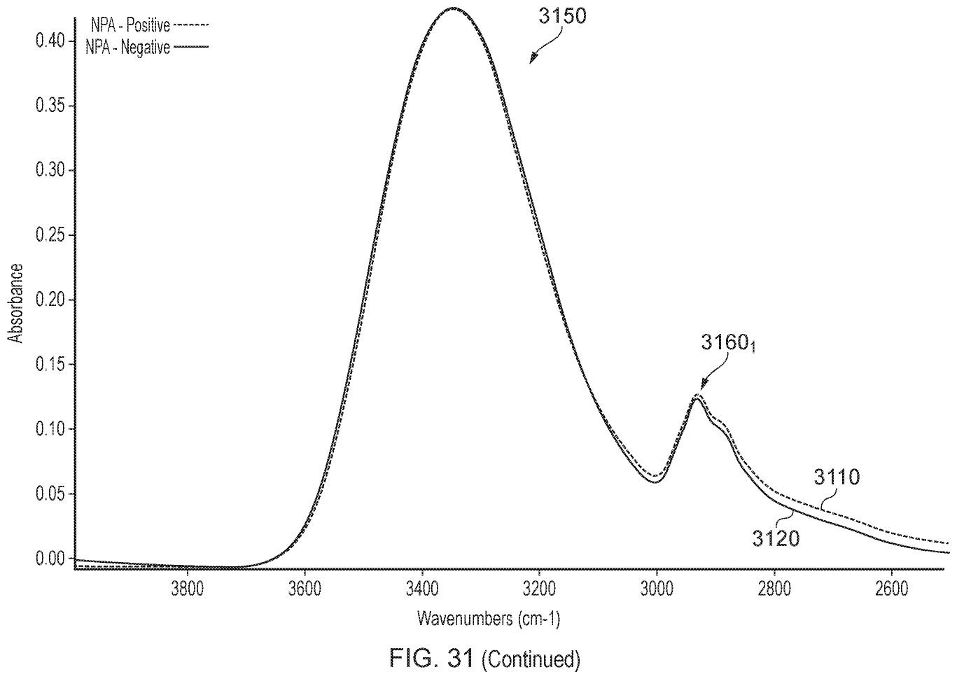

FIG. 31 illustrates a discrimination analysis 3100 and absorbance spectra 3150 for COVID positive 3110 and COVID negative 3120 nasopharyngeal samples of patients in a spectral region from 4000-2500 cm⁻¹. The discrimination analysis 3100 shows data points associated with COVID positive nasopharyngeal samples (red dots) and COVID negative nasopharyngeal samples (blue squares). To arrive at discriminant analysis 3100, the data obtained by the vibrational spectroscopy technique in the spectral region from 4000-2500 cm⁻¹ is pre-processed, so that unsupervised coupled with supervised learning techniques (PCA-LDA) can be used to visualise the data. Discriminant analysis 3100 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 3150 associated with these nasopharyngeal samples was obtained via FTIR. As can be seen in the spectra 3150, there is a peak 3160₁ where differences in the spectra taken from COVID positive/negative samples are prominent. Differences in the peak shift 3160₁ illustrates differences in the CH vibrations from lipids and proteins between spectra 3110 and 3120. By providing spectroscopic data associated with the absorbance spectra 3150 from COVID positive/negative nasopharyngeal samples within this spectral region into one or more machine learning modules, one or more trained machine learning models can be provided as described hereinabove. These trained models are able to identify if a nasopharyngeal sample is COVID positive or negative when provided with spectra from that sample in this spectral region. The spectra from all the classes associated with spectra 3150 present different shifts in both absorbance and wavenumber and these shifts allow an appropriate differentiation from all classes.

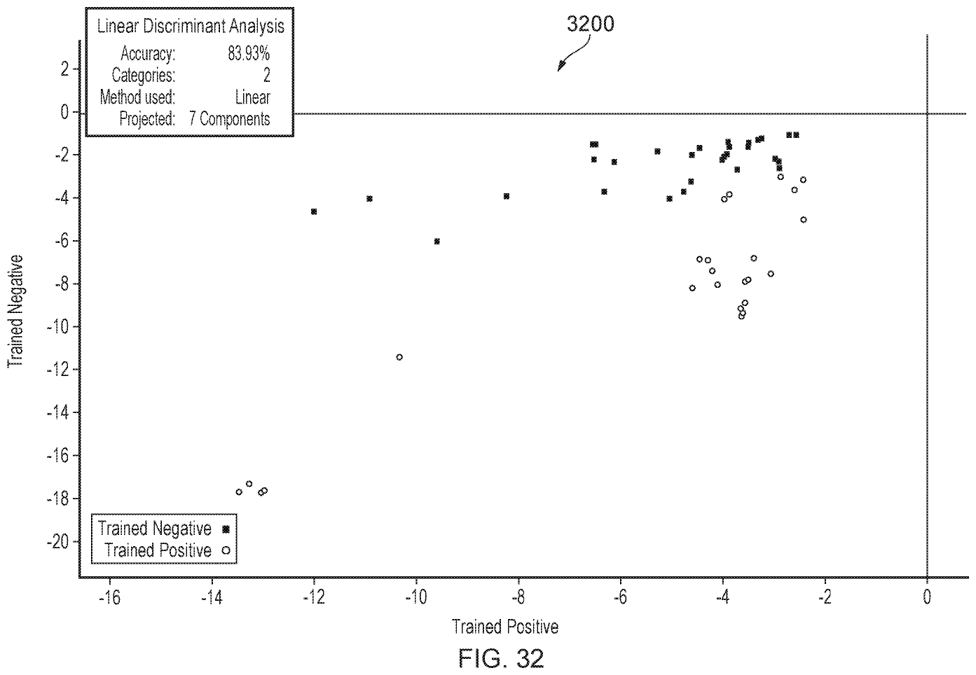

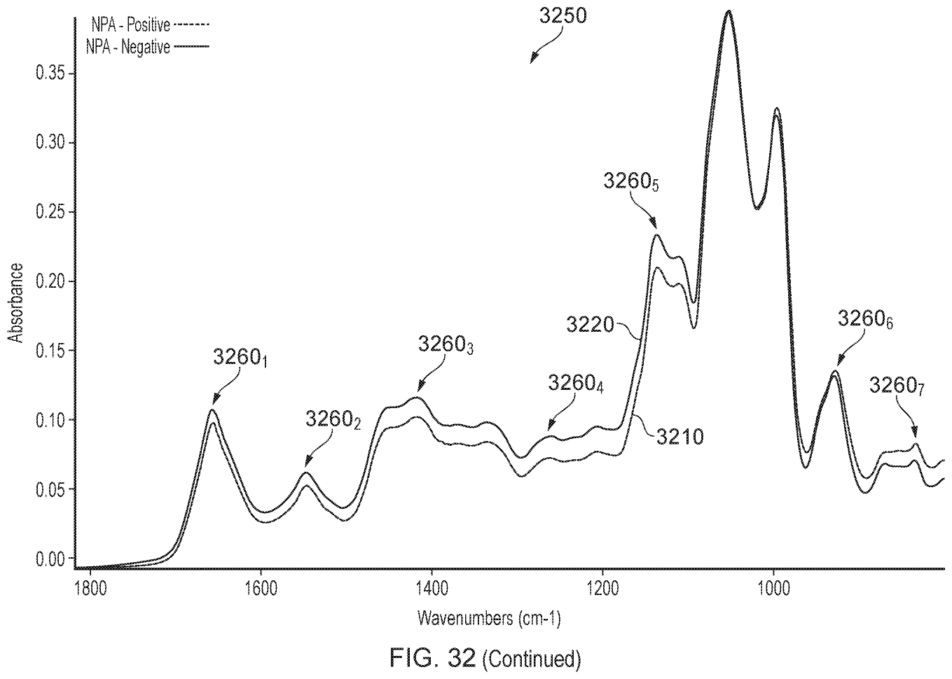

FIG. 32 illustrates a discrimination analysis 3200 and absorbance spectra 3250 for COVID positive 3210 and COVID negative 3220 nasopharyngeal samples of patients in a spectral region from 1800-400 cm⁻¹. The discrimination analysis 3200 shows data points associated with COVID positive nasopharyngeal samples (red dots) and COVID negative nasopharyngeal samples (blue squares). To arrive at discriminant analysis 3200, the data obtained by the vibrational spectroscopy technique in the spectral region from 1800-400 cm⁻¹ is pre-processed, so that unsupervised coupled with supervised learning techniques (PCA-LDA) can be used to visualise the data. Discriminant analysis 3200 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes. The closer the data sets are to the zero intersection, the more similar they are. The absorbance spectra 3250 associated with these nasopharyngeal samples was obtained via FTIR. As can be seen in the spectra 3250, there are a number of peaks 3260₁-3260₇ where differences in the spectra taken from COVID positive/negative samples are prominent. Differences between spectra 3210 and 3220 in the peak shifts 3260₁-3260₅ illustrate the differences that occur on CH₂, CO, and/or CC vibrations of polysaccharides, whereas peaks 3260₆ and 3260₇ illustrate the differences that occur in the nucleic acid spectral region. By providing spectroscopic data associated with the absorbance spectra 3250 from COVID positive/negative nasopharyngeal samples within this spectral region into one or more machine learning modules, one or more trained machine learning models can be provided as described hereinabove. These trained models are able to identify if a nasopharyngeal sample is COVID positive or negative when provided with spectra from that sample in this spectral region. The spectra from all the classes associated with spectra 3250 present different shifts in both absorbance and wavenumber and these shifts allow an appropriate differentiation from all classes.

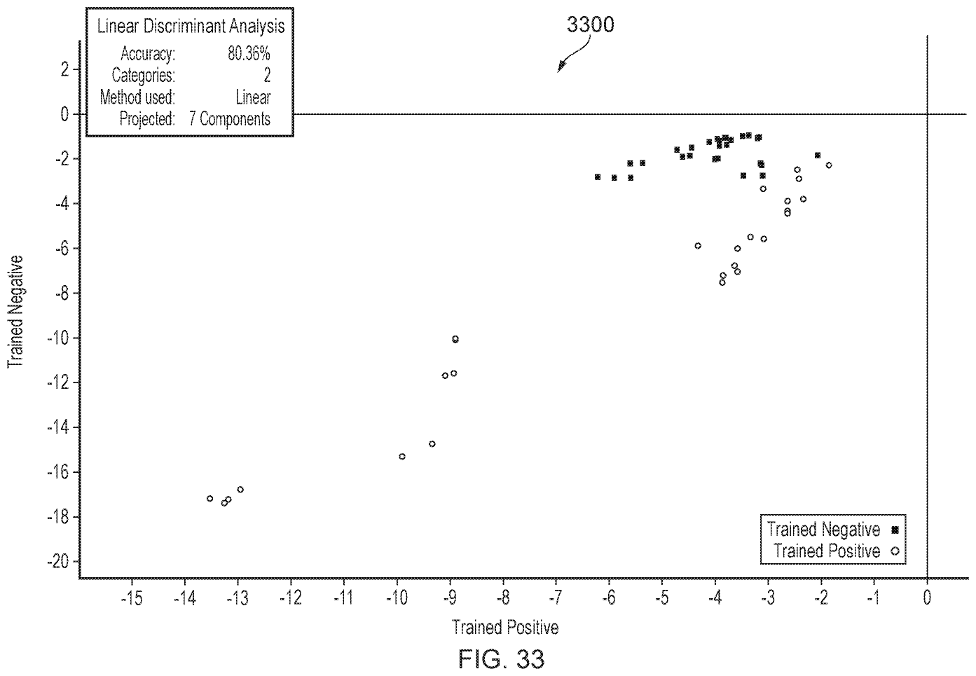

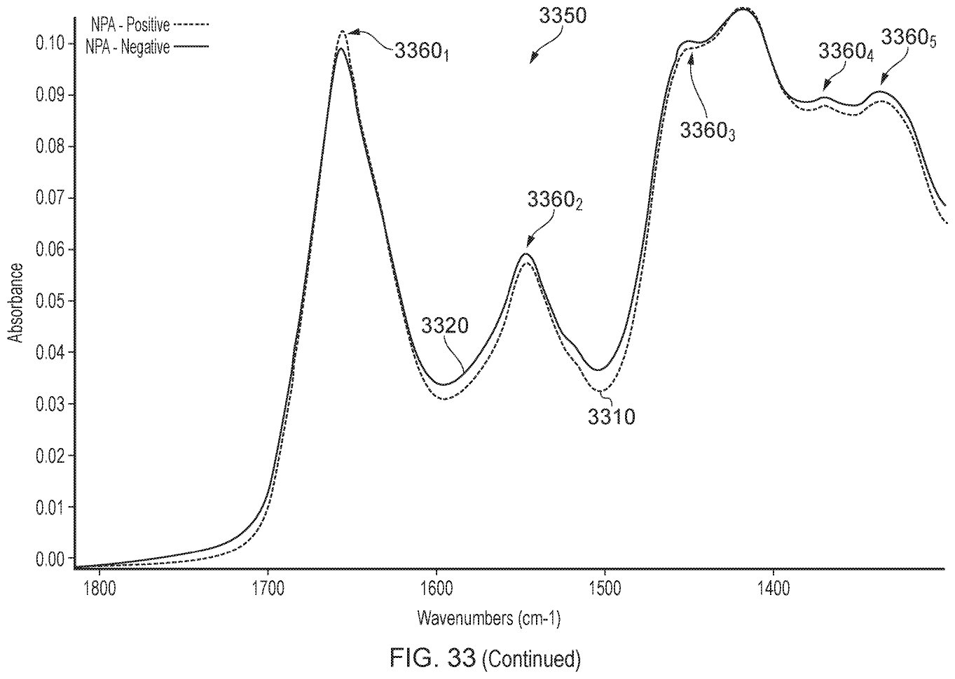

FIG. 33 illustrates a discrimination analysis 3300 and absorbance spectra 3350 for COVID positive 3310 and COVID negative 3320 nasopharyngeal samples of patients in a spectral region from 1800-1300 cm⁻¹. The discrimination analysis 3300 shows data points associated with COVID positive nasopharyngeal samples (red dots) and COVID negative nasopharyngeal samples (blue squares). To arrive at discriminant analysis 3300, the data obtained by the vibrational spectroscopy technique in the spectral region from 1800-1300 cm⁻¹ is pre-processed, so that unsupervised coupled with supervised learning techniques (PCA-LDA) can be used to visualise the data. Discriminant analysis 3300 enables observation of a clear segregation of the data points associated with a COVID positive/negative specimen, thus showing that the clusters of data points are different from each other and allowing data prediction. The score obtained from the PCA-LDA treatment allows to allocate the spectral points into the plane, allowing a differentiation of classes.

The closer the data sets are to the zero intersection, the more similar they are. The absorbance spectra 3350 associated with these nasopharyngeal samples was obtained via FTIR. As can be seen in the spectra 3350, there are a number of peaks 3360₁-3360₅ where differences in the spectra taken from COVID positive/negative samples are prominent. Differences between spectra 3310 and 3320 in the peak shifts 3360₁-3360₅ illustrate amide regions. By providing spectroscopic data associated with the absorbance spectra 3350 from COVID positive/negative nasopharyngeal samples within this spectral region into one or more machine learning modules, one or more trained machine learning models can be provided as described hereinabove. These trained models are able to identify if a nasopharyngeal sample is COVID positive or negative when provided with spectra from that sample in this spectral region. The spectra from all the classes associated with spectra 3350 present different shifts in both absorbance and wavenumber and these shifts allow an appropriate differentiation from all classes.

As described above, performing a discriminative analysis of the data obtained allows the pre-visualisation of the data to be carried out as a preliminary support for the modelling of machine learning models. The visualisation of clusters in different spectral regions not only allows the feasibility and effectiveness in regions other than the full wavenumber region to be observed, but having identified short regions can also help in the creation of vibrational spectroscopic devices with a lower complexity, both structurally and in terms of size.

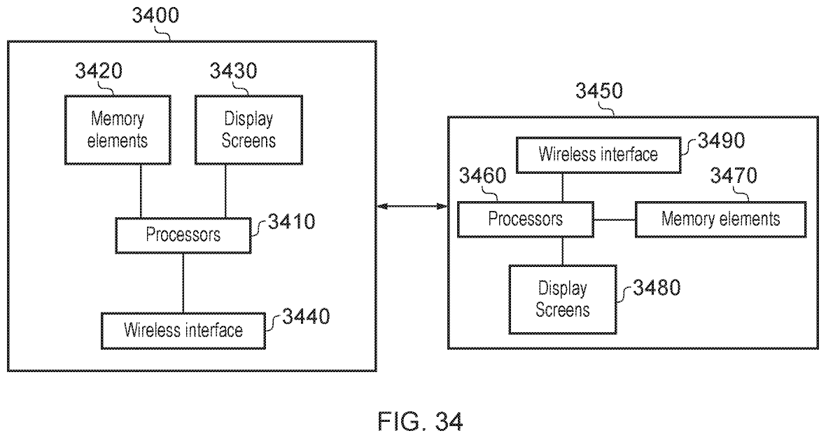

FIG. 34 illustrates a spectrometer 3400 having one or more processors 3410, at least one memory element 3420 (or a non-transitory computer readable storage medium), one or more display screens 3430, and a wireless interface 3440. Also shown in FIG. 34 is a computing device 3450 having one or more processors 3460, one or more memory elements 3470, one or more display screens 3480, and a wireless interface 3490. The computing device 3450 may be a PC, laptop, mobile phone, tablet computer, server (e.g., a cloud-based server), or the like. As will be appreciated by the person of skill in the art, any wireless communication methodology may be used for communicating between the spectrometer and the computing device. Additionally or alternatively, a wired connection may be utilised. The spectrometer 3400 may be a Raman or IR spectrometer which collects spectroscopic data associated with a biological sample under inspection. The spectroscopic data may be stored in memory elements 3420 and/or may be transmitted to computing device for storage in memory elements 3470. Memory elements 3420 and/or memory elements 3470 may also store one or more trained machine learning models and/or one or more untrained machine learning models. The memory elements store these models as computer-readable instructions that are executable by the processors 3410, 3460. When the models need to be executed, they may be executed on the processors 3410, 3470 from local memory elements and/or they may be transmitted from memory elements via the wireless interfaces 3440, 3490 to be executed remotely on corresponding processors. As an example, when spectroscopic data is obtained by the spectrometer 3400, this data may be transmitted to computing device 3450 where it is input into one or more trained machine learning models stored in memory elements 3470 and configured to execute on processors 3460. This enables identification of at least one microorganism present in the biological sample under inspection. The display screens 3430, 3490 are configured to display spectra obtained by the spectrometer 3400 and/or to display the identification results output by one or more trained machine learning models when provided with spectroscopic data.

1. A method of training at least one machine learning module to provide at least one trained machine learning model that is configured to identify at least one microorganism in a biological sample, the method comprising the steps of:
providing spectroscopic data associated with at least one microorganism, and obtained via a Raman or Infrared spectroscopy technique, as an input into at least one machine learning module; and
responsive to providing the spectroscopic data, providing at least one trained machine learning model,
wherein the trained machine learning model is configured to identify at least one microorganism in a biological sample.

2. The method as claimed in claim 1, wherein the spectroscopic data is obtained via an Infrared spectroscopy technique, and wherein the Infrared spectroscopy technique is a Fourier transform infrared spectroscopy technique coupled with an attenuated total reflectance spectroscopy technique.

3. The method as claimed in claim 1, wherein the at least one microorganism comprises a virus, and wherein optionally the virus is resistant to antivirals.

4. The method as claimed in claim 1, further comprising:
placing the biological sample onto a CaF₂ substrate; and
providing the biological sample on the CaF₂ substrate into a Raman or Infrared spectrometer to obtain the spectroscopic data.

5. The method as claimed in claim 1, further comprising:
providing the biological sample into a Raman spectrometer with an attached microscope objective lens to obtain Raman spectroscopic data and/or into an Infrared spectrometer with an attached microscope objective lens to obtain Infrared spectroscopic data.

6. The method as claimed in claim 1, wherein the at least one microorganism comprises an antibiotic resistant microorganism.

7. The method as claimed in claim 1, further comprising:
providing spectroscopic data within a first spectral region from 4000-400 cm⁻¹ as an input into at least one machine learning module, and responsive to providing the spectroscopic data within the first spectral region, providing at least one trained machine learning model associated with the first spectral region.

8. The method as claimed in claim 7, further comprising:
providing spectroscopic data within a second spectral region from 1800-400 cm⁻¹ as an input into at least one machine learning module, and responsive to providing the spectroscopic data within the second spectral region, providing at least one trained machine learning model associated with the second spectral region; and/or
providing spectroscopic data within a third spectral region from 1810-1700 cm⁻¹ as an input into at least one machine learning module, and responsive to providing the spectroscopic data within the third spectral region, providing at least one trained machine learning model associated with the third spectral region; and/or
providing spectroscopic data within a fourth spectral region from 1590-1290 cm⁻¹ as an input into at least one machine learning module, and responsive to providing the spectroscopic data within the fourth spectral region, providing at least one trained machine learning model associated with the fourth spectral region; and/or
providing spectroscopic data within a fifth spectral region from 1600-1500 cm⁻¹ as an input into at least one machine learning module, and responsive to providing the spectroscopic data within the fifth spectral region, providing at least one trained machine learning model associated with the fifth spectral region.

9. The method as claimed in claim 1, wherein each machine learning module is a multinomial classifier.

10. The method as claimed in claim 1, wherein each machine learning module is one of a linear discriminant analysis module, a support vector machine module, a logistic regression module, a K nearest neighbours module, a random forest module, an artificial neural network module or a convolutional neural network module.

11. The method as claimed in claim 1, further comprising:
providing spectroscopic data as an input into a plurality of machine learning modules; and
responsive to providing the spectroscopic data, providing a plurality of trained machine learning models,
wherein each trained machine learning model is configured to identify at least one microorganism in a biological sample.

12. The method as claimed in claim 11, wherein the plurality of machine learning modules comprises a linear discriminant analysis module, a support vector machine module, a logistic regression module, a K nearest neighbours module, a random forest module, optionally an artificial neural network module, and optionally a convolutional neural network module; the method further comprising:
providing spectroscopic data as an input into each of the plurality of machine learning modules; and
responsive to providing the spectroscopic data, providing a trained linear discriminant analysis based model, a trained support vector machine based model, a trained logistic regression based model, a trained K nearest neighbours based model, a trained random forest based model, optionally a trained artificial neural network based model and optionally a trained convolutional neural network based model.

13. The method as claimed in claim 1, wherein the at least one trained machine learning model is configured to identify a plurality of microorganisms.

14. The method as claimed in claim 1, further comprising:
providing the spectroscopic data as a feature matrix comprising a plurality of instances, each instance comprising spectroscopic data.

15. The method as claimed in claim 14, wherein the plurality of instances comprises intensity values, associated with spectroscopic measurements of a plurality of microorganisms, at each of a plurality of wavelengths or wavenumbers.

16. The method as claimed in claim 14, wherein each instance comprises intensity values, associated with a spectroscopic measurement of a predetermined microorganism, at each of a plurality of wavelengths or wavenumbers.

17. The method as claimed in claim 14, wherein each instance of the feature matrix further comprises an encoded class label that indicates a type of microorganism associated with the spectroscopic data in that instance.

18. The method as claimed in claim 1, further comprising:
responsive to providing the spectroscopic data, determining one or more bio-markers associated with at least one predetermined microorganism via the machine learning module, wherein the trained machine learning model is at least partly based on the determined bio-markers;
the bio-markers including at least one of a spectral peak position, a spectral shift, a spectral shape, a spectral intensity, and a spectral area associated with a predetermined microorganism.

19. The method as claimed in claim 1, wherein the biological sample is at least one bodily fluid of a patient that includes the at least one microorganism.

20. The method as claimed in claim 1, wherein the microorganism is:
a bacterial pathogen contained in one of the following Phylum: Actinobacteria, Bacteroidetes, Firmicutes, Fusobacteria, Proteobacteria; and/or
a viral pathogen contained in one of the following orders: Herpesvirales, Mononegavirales, Nidovirales, Picornavirales; and/or
a viral pathogen contained in one of the following families: Adenovirus, Astroviridiae, Caliciviridiae, Flaviviridiae, Hepadnaviridae, Hepeviridiae, Orthomyxoviridiae, Reoviridiae, Coronaviridae; and/or
a fungal pathogen contained in one of more of the divisions: Ascomycota, Basidiomycota; and/or
at least one of:
Staphylococcus spp and/or associated serotypes;
Klebsiella spp and/or associated serotypes;
Streptococcus spp and/or associated serotypes;
Pseudomonas spp and/or associated serotypes;
Candida spp and/or associated serotypes;
Escherichia spp and/or associated serotypes;
Saccharomyces spp and/or associated serotypes;
Salmonella spp and/or associated serotypes;
Vibrio spp and/or associated serotypes;
Enterococcus spp and/or associated serotypes; and
SARS-COV-2 spp.

21. A method of identifying at least one microorganism in a biological sample, comprising the steps of:
providing spectroscopic data associated with a biological sample, and obtained via a Raman or Infrared spectroscopy technique, as an input into at least one trained machine learning model; and
responsive to providing the spectroscopic data, identifying at least one microorganism in the biological sample.

22. The method as claimed in claim 21, further comprising:
providing the spectroscopic data associated with the biological sample as an input into the at least one trained machine learning model trained via the method as claimed in any of claims 1 to 20.

23. The method as claimed in claim 21, further comprising:
providing the biological sample into a portable Raman spectrometer, optionally with an attached microscope objective lens, to obtain Raman spectroscopic data and/or into a portable Infrared spectrometer, optionally with an attached microscope objective lens, to obtain Infrared spectroscopic data.

24. The method as claimed in claim 21, wherein the spectroscopic data is obtained via an Infrared spectroscopy technique, and wherein the Infrared spectroscopy technique is a Fourier transform infrared spectroscopy technique coupled with an attenuated total reflectance spectroscopy technique.

25. The method as claimed in claim 21, further comprising:
identifying the at least one microorganism as an antibiotic resistant microorganism or a virus resistant to antivirals.

26. The method as claimed in claim 21, further comprising:
placing the biological sample onto a CaF₂ substrate; and
providing the biological sample on the CaF₂ substrate into a Raman or Infrared spectrometer to obtain the spectroscopic data.

27. The method as claimed in claim 21, further comprising:
providing spectroscopic data within a first spectral region from 4000-400 cm⁻¹ as an input into the at least one trained machine learning model, and responsive to providing the spectroscopic data within the first spectral region, identifying at least one microorganism in the biological sample.

28. The method as claimed in claim 27, further comprising:
providing spectroscopic data within a second spectral region from 1800-400 cm⁻¹ as an input into the at least one trained machine learning model, and responsive to providing the spectroscopic data within the second spectral region, identifying at least one microorganism in the biological sample; and/or
providing spectroscopic data within a third spectral region from 1810-1700 cm⁻¹ as an input into the at least one trained machine learning model, and responsive to providing the spectroscopic data within the third spectral region, identifying at least one microorganism in the biological sample; and/or
providing spectroscopic data within a fourth spectral region from 1590-1290 cm⁻¹ as an input into the at least one trained machine learning model, and responsive to providing the spectroscopic data within the fourth spectral region, identifying at least one microorganism in the biological sample; and/or
providing spectroscopic data within a fifth spectral region from 1600-1500 cm⁻¹ as an input into the at least one trained machine learning model, and responsive to providing the spectroscopic data within the fifth spectral region, identifying at least one microorganism in the biological sample.

29. The method as claimed in claim 21, further comprising:
providing the spectroscopic data as an input into each of a plurality of trained machine learning models; and
responsive to providing the spectroscopic data, identifying at least one microorganism via each of the plurality of trained machine learning models.

30. The method as claimed in claim 29, further comprising:
providing the spectroscopic data as an input into each of the plurality of trained machine learning models simultaneously.

31. The method as claimed in claim 21, wherein each trained machine learning model is a multinomial classifier.

32. The method as claimed in claim 21, further comprising:
responsive to providing the spectroscopic data, identifying a plurality of microorganisms in the biological sample.

33. The method as claimed in claim 21, wherein the biological sample is at least one bodily fluid of a patient that includes the at least one microorganism.

34. The method as claimed in claim 21, wherein the biological sample comprises a mixture of at least two bacteria or a mixture of at least one bacterium and at least one fungus or a mixture of at least two fungi or a mixture of at least one bacterium and at least one virus or a mixture of at least one fungus and at least one virus or a bacterial and fungal species present as a colony, and optionally the biological sample is at least one bacterial or fungal culture derived from a sample from a patient that includes the at least one microorganism, and further optionally the biological sample comprises a mixture of at least two bacteria or a mixture of at least one bacterium and at least one fungus or a mixture of at least two fungi or a mixture of at least one bacterium.

35. Apparatus comprising at least one memory for storing spectroscopic data and at least one processor, communicatively coupled to the memory, and configured to perform the steps of the method as claimed in claim 1.

36. A system comprising at least one memory for storing spectroscopic data and at least one processor, communicatively coupled to the memory, and configured to perform the steps of the method as claimed in claim 1.

37. A computer program comprising instructions which, when the program is executed by a computer, cause the computer to carry out the steps of the method as claimed in claim 1.

38. A computer-readable data carrier having stored thereon the computer program of claim 37.
